(19) United States
(12) Patent Application Publication
(10) Pub. No.: US 2025/0154247 A1
(43) Pub. Date: May 15, 2025

(54) CANCER TREATMENT TARGETING DLL3

(71) Applicant: AMGEN INC., Thousand Oaks, CA (US)

(72) Inventors: Nooshin Hashemi Sadraei, Thousand Oaks, CA (US); Mukul Minocha, Thousand Oaks, CA (US); Amanda Goldrick, Thousand Oaks, CA (US); Xi Chen, Thousand Oaks, CA (US); Mira Kistler, Thousand Oaks, CA (US)

(21) Appl. No.: 18/836,031

(22) PCT Filed: Feb. 22, 2023
(86) PCT No.: PCT/US23/63010

§ 371 Date: Aug. 6, 2024

Related U.S. Application Data
(60) Provisional application No. 63/429,311, filed on Dec. 1, 2022.
(60) Provisional application No. 63/313,119, filed on Feb. 23, 2022.

(51) Int. Cl. C07K 16/28; A61K 39/00; A61P 35/00

(52) U.S. Cl. CPC C07K 16/28; A61P 35/00; A61K 2039/505; A61K 2039/545; C07K 2317/92; C07K 2317/94

(57) ABSTRACT

Invention disclosed herein provides a method for the treatment of DLL3-positive cancer or SCLC, comprising administering to a subject in need thereof an anti-DLL3 agent alone, or in combination with an anti-PD-L 1 antibody and/or chemotherapeutic agents. Step dosing or extended IV infusion of the anti-DLL3 agent is also disclosed.

### CROSS REFERENCE OF RELATED APPLICATION

This application claims the benefit of U.S. Provisional Application Nos. 63/313,119 and 63/429,311, filed on Feb. 23, 2022 and Dec. 1, 2022, respectively. The content of which is incorporated in its entirety by reference herein.

### SUBMISSION OF SEQUENCE ON XML FILE

The context of the following submission on XML files is incorporated herein by reference in its entirety: 43,375 byte XML file named “10009-WO01-SEC_SequenceListing”; created on Feb. 13, 2023.

### FIELD OF THE INVENTION

The present application relates to dosage and administration of cancer therapy targeting DLL3, including combination therapy with agents targeting PD-L1 and/or chemotherapy agents.

### BACKGROUND OF THE INVENTION

Delta-like 3 (DLL3) is a type 1 transmembrane protein and noncanonical Notch ligand. DLL3 is a promising target for the development of T-cell therapies due to its high expression on the cell surface of neuroendocrine tumors, and minimal, mainly cytoplasmic localization in normal tissues (Owen et al., J Hematol Oncol., 12:61 (2019)). Neuroendocrine tumors typically begin in neuroendocrine cells and can occur in organs such as the lungs, appendix, small intestine, rectum and pancreas. Small cell lung cancer (SCLC) is a neuroendocrine cancer wherein DLL3 is differentially expressed. Using immunohistochemistry (IHC), 85% of SCLC tumors stained positive for DLL3 in a pattern consistent with both membranous and cytoplasmic expression. In contrast, low levels of DLL3 protein expression were detected in normal brain, pancreatic islets, and pituitary gland with a cytoplasmic staining pattern (Saunders et al, Sci Transl Med. 7:302ra136 (2015)).

SCLC is an aggressive form of lung cancer with a poor prognosis and limited therapeutic options and represents about 10-15% of lung cancers. Survival rates have remained low for several decades, with only 5% of SCLC patients surviving five years, in a large part due to the lack of new therapies to combat this form of lung cancer. SCLC is characterized by neuroendocrine differentiation, a high growth fraction, rapid doubling time and early establishment of widespread metastatic lesions. About a third of patients present with limited stage disease. Most patients present with extensive-stage disease. These stages impact available therapeutic regiments, with limited stage disease treated with chemotherapy and radiation and extensive stage disease treated with chemotherapy alone.

Patients with SCLC have high rates of response to first line chemotherapy, which includes etoposide and cisplatin, and also to radiation therapy, but invariably quickly relapse. There are approved therapies for relapsed disease but chemoresistance develops for which no therapeutic options are available. Prognosis in the relapsed refractory setting is extremely poor, with rapid disease progression and short median survival of less than six months once the patient is on the third line of treatment. Patients with extensive stage SCLC (ES-SCLC) develop drug resistance and die as a result of disease at a median time of 10 to 12 months from diagnosis

AMG 757 (also known as tarlatamab) is a bispecific T-cell engager (BiTE®) molecule targeting DLL3 on cancer cells and CD3 on T-cells. It is developed for the treatment of DLL3-positive cancers such as SCLC and neuroendocrine prostate cancer (NEPC) and is being evaluated in clinical trials.

Efforts have been made to develop therapies for the treatment of DLL3-positive such as SCLC, but overall survival remains poor. There is an unmet medical need for the development of therapies for the treatment of DLL3 positive cancers such as SCLC.

### SUMMARY OF THE INVENTION

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1: A method of treating DLL3-positive cancer, comprising administering to a subject in need thereof an anti-DLL3 agent, wherein the anti-DLL3 agent is administered at a dose of from 10 mg to 100 mg weeks twice every three weeks.

E2: A method of treating DLL3-positive cancer, comprising administering to a subject in need thereof an anti-DLL3 agent, wherein the anti-DLL3 agent is administered at a dose of from 20 mg to 200 mg once every three weeks.

E3: A method of treating DLL3-positive cancer comprising administering to a subject in need thereof an anti-DLL3 agent, wherein the anti-DLL3 agent is administered according to the following regimen: a) administering the anti-DLL3 agent in a first cycle wherein (i) the anti-DLL3 agent is administered at a dose of from 1 mg to 200 mg by continuous intravenous infusion over a period of 2 days to 7 days, and (ii) after the continuous intravenous infusion, the anti-DLL3 agent is administered by a bolus intravenous infusion on day 8, day 15, or both day 8 and day 15, and b) administering the anti-DLL3 agent according to any one of i) to iii): i) administering one or more subsequent doses of the anti-DLL3 agent at a dose of from 10 mg to 100 mg starting on day 29 and once every two weeks thereafter; ii) administering one or more subsequent doses of the anti-DLL3 agent at a dose of from 10 mg to 100 mg starting on day 22 and twice every three weeks thereafter; and iii) administering one or more subsequent doses of the anti-DLL3 agent at a dose of from 20 mg to 200 mg starting on day 22 and thereafter once every three weeks.

E4: A method of treating DLL3-positive cancer, comprising administering to a subject in need thereof an anti-DLL3 agent, an anti-PD-L1 antibody, and optionally one or more chemotherapeutic agents, wherein the anti-DLL3 agent is administered according to any one of a) to c): a) administering the anti-DLL3 agent at a dose of from 10 mg to 100 mg once every two weeks; b) administering the anti-DLL3 agent at a dose of from 10 mg to 100 mg twice every three weeks; and c) administering the anti-DLL3 agent at a dose of from 20 mg to 200 mg once every three weeks.

E5: The method of any one of E1 to E4, wherein the anti-DLL3 positive cancer is small cell lung cancer (SCLC).

E6: The method of any one of E1-E5, wherein the anti-DLL3 positive cancer is Relapsed/refractory (RR) SCLC or Extensive disease (ED) SCLC.

E7: The method of any one of E1-E6, wherein the anti-DLL3 agent is a bispecific T cell engaging antigen-binding polypeptide comprising two binding domains: the first domain binds to human DLL3, and the second domain binds to human CD3.

E8: The method of E7, wherein the DLL3-binding domain binds to an epitope of human DLL3 comprised within the amino acid sequence of SEQ ID NO:29.

E9: The method of E7 or E8, wherein the DLL3-binding domain comprises (a) a heavy chain variable region (VH) that comprises: (i) a VH complementarity determining region one (CDR-H1) comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and (b) a light chain variable region (VL) that comprises: (i) a VL complementarity determining region one (CDR-L1) comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6.

E10: The method of any one of E7-E9, wherein the DLL3-binding domain comprises: (1) a VH that comprises the amino acid sequence of SEQ ID NO:7, and a VL that comprises the amino acid sequence of SEQ ID NO:8, or (2) a VH that comprises the amino acid sequence of SEQ ID NO:11, and a VL that comprises the amino acid sequence of SEQ ID NO:12.

E11: The method of any one of E7-E10, wherein the VH and VL of the DLL3-binding domain are joined by a linker to form a single chain Fv (scFv).

E12: The method of any one of E7-E11, wherein the DLL3-binding domain comprises the amino acid sequence of SEQ ID NO:9 or SEQ ID NO:13.

E13: The method of any one of E7-E12, wherein the CD3-binding domain comprises: (a) a VH that comprises: a CDR-H1 comprising the amino acid sequence of SEQ ID NO:18, a CDR-H2 comprising the amino acid sequence of SEQ ID NO:19, and a CDR-H3 comprising the amino acid sequence of SEQ ID NO:20; and a VL that comprises: a CDR-L1 comprising the amino acid sequence of SEQ ID NO: 15, a CDR-L2 comprising the amino acid sequence of SEQ ID NO:16, and a CDR-L3 comprising the amino acid sequence of SEQ ID NO:17.

E14: The method of any one of E7-E13, wherein the CD3-binding domain comprises: a VH that comprises the amino acid sequence of SEQ ID NO:21, and a VL that comprises the amino acid sequence of SEQ ID NO:22.

E15: The method of E13 or E14, wherein the VH and VL of the CD3-binding domain are joined by a linker to form a single chain Fv (scFv).

E16: The method of any one of E13-E15, wherein the CD3-binding domain comprises the amino acid sequence of SEQ ID NO:23.

E17: The method of any one of E7-E16, wherein the DLL3-binding domain and the CD3-binding domain are joined by a linker.

E18: The method of any one of E7-E17, the anti-DLL3 agent is a bispecific T cell engaging antigen-binding polypeptide comprising a DLL3-binding domain and a CD3-binding domain. The DLL3-binding domain comprises (a) a heavy chain variable region (VH) that comprises: (i) a VH complementarity determining region one (CDR-H1) comprising the amino acid sequence of SEQ ID NO: 1; (ii) a CDR-H2 comprising the amino acid sequence of SEQ ID NO:2; and (iii) a CDR-H3 comprising the amino acid sequence of SEQ ID NO:3; and (b) a light chain variable region (VL) that comprises: (i) a VL complementarity determining region one (CDR-L1) comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6. The CD3-binding domain comprises (a) a VH that comprises: (i) a CDR-H1 comprising the amino acid sequence of SEQ ID NO: 18, (ii) a CDR-H2 comprising the amino acid sequence of SEQ ID NO:19, and (iii) a CDR-H3 comprising the amino acid sequence of SEQ ID NO:20; and (b) a VL that comprises: (i) a CDR-L1 comprising the amino acid sequence of SEQ ID NO:15, (ii) a CDR-L2 comprising the amino acid sequence of SEQ ID NO:16, and (iii) a CDR-L3 comprising the amino acid sequence of SEQ ID NO:17.

E19: The method of any one of E7-E18, the DLL3-binding domain comprises a VH that comprises the amino acid sequence of SEQ ID NO:7, and a VL that comprises the amino acid sequence of SEQ ID NO:8, and the CD3-binding domain comprises a VH that comprises the amino acid sequence of SEQ ID NO:21, and a VL that comprises the amino acid sequence of SEQ ID NO:22.

E20: The method of any one of E7-E18, the DLL3-binding domain comprises a VH that comprises the amino acid sequence of SEQ ID NO:11, and a VL that comprises the amino acid sequence of SEQ ID NO:12, and the CD3-binding domain comprises a VH that comprises the amino acid sequence of SEQ ID NO:21, and a VL that comprises the amino acid sequence of SEQ ID NO:22.

E21: The method of any one of E7-E19, wherein the DLL3-binding domain comprises the amino acid of SEQ ID NO:9 and the CD3-binding domain comprises the amino acid of SEQ ID NO: 23.

E22: The method of any one of E7-E18 or E20, the DLL3-binding domain comprises the amino acid of SEQ ID NO:13 and the CD3-binding domain comprises the amino acid of SEQ ID NO:23.

E23: The method of E21 or E22, wherein the anti-DLL3 agent comprises the amino acid sequence of SEQ ID NO: 10 or SEQ ID NO: 14.

E24: The method of any one of E7-E23, wherein the anti-DLL3 agent comprises the amino acid of SEQ ID NO:27 or SEQ ID NO: 32.

E25: The method of any one of E1-E24, wherein the method further comprises administering one or more additional therapeutic agent to the subject.

E26: The method of E25, wherein the one or more additional therapeutic agents is a corticosteroid (e.g., dexamethasone), saline, or an anti-IL-6 antibody.

E27: The method of any one of E25 or E26, wherein the one or more additional therapeutic agent is administered to the subject in the first cycle wherein the anti-DLL3 agent is administered.

E28: The method of any one of E1-E27, wherein the anti-DLL3 agent is prepared by a process wherein a host cell comprising a nucleic acid encoding the anti-DLL3 agent described in any one of E7-E24 is cultured under conditions allowing the expression of the anti-DLL3 agent and the expressed anti-DLL3 agent is then recovered from the cell culture.

E29: The method of any one of E1-E28, wherein the subject is a human. E30: The method of E29, wherein the subject had at least one prior treatment of the cancer and relapsed, for example, two or more prior treatment and relapsed. E31: The method of E30, wherein the least one prior treatment of the cancer is platinum, chemotherapy, etoposide, and optionally an anti-PD-L1 antibody. E32: The method of E29, wherein the subject had no prior systemic treatment of the cancer. E33: The method of any one of E1-32, wherein the anti-DLL3 agent is tarlatamab. E34: The method of any one of E3, wherein the anti-PD-L1 antibody is atezolizumab or durvalumab.

E33: An anti-DLL3 agent for use in the treatment of DLL3-positive cancer (e.g., SCLC), wherein the anti-DLL3 agent is administered as set forth in any one of embodiments E1-E29.

### BRIEF DESCRIPTION OF THE DRAWINGS

FIG. 1A is a graph showing the best percent change from baseline in tumor burden (defined by the sum of the longest diameters (SLD) of all target lesions) in 94 patients whose data cut-off date was at least 9 weeks after the first dose date and for whom postbaseline tumor data were available. CR denotes complete response, PR partial response, SD stable disease, and NE not evaluable. SD\hat{ } indicates patients had an initial response but did not have confirmation of response on the subsequent scan and PR** indicates patients had an initial PR and still have potential for future confirmative scans. One confirmed subject in cohort 30 had missing sum of diameters for lesion measurement and was not included in the plot. T Step dosing (i.e., 1 mg run-in dose) was used in these cohorts. FIG. 1B is a graph showing the time to response, the duration of treatment, and patient status as of the data cutoff date according to dose of tarlatamab for all patients with confirmed response (N=25).

FIG. 2A shows a Kaplan-Meier curve of progression-free survival for patients whose data cut-off date was at least 9 weeks after the first dose date (N=107). FIG. 2B shows a Kaplan-Meier curve of overall survival for patients whose data cut-off date was at least 9 weeks after the first dose date (N=107).

FIGS. 3A-3D are graphs showing peak cytokine levels (6A: IL-6; 6B: IL-8; 6C: IL-10; 6D: TNF-α) trended higher in patients with CRS as compared to patients without CRS. Biomarker evaluable patients (N=86); patients with any grade CRS in Cycle 1 (n=45); patients without CRS (n=40). C1, Cycle 1; CRS, cytokine release syndrome; G, grade.

FIG. 4 is a graph showing a longitudinal analysis of IL-10 expression in patients from the phase I study described in Example 1. IL-10 showed significant elevation above referenced normal range, and was higher in patients with CRS. JT Trend test adjusted P-value=0.049 (significant at 95% confidence); KW test of association adjusted P-value=0.096 (significant at 90% confidence; not significant at 95% confidence). Yellow dotted lines indicate referenced normal range.

FIGS. 5A and 5B are graphs showing analysis of IFN-γ expression in patients from the phase I study described in Example 1. IFN-γ induction was above the physiologic range; induction was similar between those with versus without Cycle 1 CRS. JT Trend test adjusted P-value=0.234 (not significant at 95% confidence); KW test of association adjusted P-value=0.317 (not significant at 90% or at 95% confidence). Yellow dotted lines indicate referenced normal range.

FIG. 6 is a schematic diagram of the clinical study described in Example 5.

### DETAILED DESCRIPTION

AMG 757 is a half-life-extended BiTE® (bispecific T cell engager) molecule developed for the treatment of DLL3-positive cancers such as SCLC. The activity of AMG 757 requires the simultaneous binding to both target cells (DLL3⁺cells) and T cells. The pharmacological effect of AMG 757 is mediated by specific redirection of previously primed cytotoxic CD8⁺ or CD4⁺ T lymphocytes to kill DLL3⁺ cells. AMG 757 is being evaluated in a first-in-human study in subjects with SCLC (Study 20160323) and was found to have anti-tumor activity starting at dose level of 0.3 mg once every two weeks (Q2W) and with acceptable safety at doses up to 100 mg Q2W.

In pre-clinical studies, treatment with AMG 757 induces upregulation of PD-1 and programmed death ligand 1 (PD-L1) on T cells. The combination of AMG 757 and anti-PD-1 antibodies increases T-cell mediated redirected lysis of tumor cells that express DLL3 compared to AMG 757 alone (Amgen Study Report R20190104). Upregulation of PD1/PD-L1 in the tumor microenvironment may be a mechanism of resistance to BiTE therapy that treatment with anti-PD1 or anti-PD-L1 therapy may mitigate.

Combination therapies including BiTE molecules and cytotoxic chemotherapies are novel for the treatment of solid tumors. As disclosed and exemplified herein, Phase 1 clinical studies were conducted for the treatment of SCLC, using agents that target DLL3 (e.g., AMG 757) alone or in combination with anti-PD-L1 agents and/or chemotherapeutic agents. Methods disclosed herein can provide improved convenience, flexibility and efficacy and/or reduce adverse effects to patients compared to once every two week dosing regimens.

### 1. Definition

Some of exemplary bispecific anti-DLLS agents disclosed herein (such as BiTE® molecules) are bispecific T cell engaging antigen-binding polypeptides. These polypeptides are recombinant proteins comprising two binding domains, each domain derived from an antigen-binding fragment of a full-length antibody. Such antigen-biding fragment retains the ability to specifically bind to an antigen (preferably with substantially the same binding affinity). Examples of an antigen-binding fragment includes (i) a Fab fragment, a monovalent fragment consisting of the VL, VH, CL and CH1 domains; (ii) a F(ab′)₂ fragment, a bivalent fragment comprising two Fab fragment is linked by a disulfide bridge at the hinge region; (iii) a Fd fragment consisting of the VH and CH1 domains; (iv) a Fv fragment consisting of the VL and VH domains of a singe arm of an antibody, and (vs) a dAb fragment (Ward et al., 1989 Nature 341:544-546), which consists of a VH domain. Furthermore, although the two domains of the Fv fragment, VL and VH are coded for b separate genes they can be joined, using recombinant methods, by a synthetic linker that enables them to be made as a single protein chain in which the VL and VH regions pair to form monovalent molecules (known as single chain Fv (scFv; see e.g., Bird et al. Science 242:423-426 (1988) and Huston et al., 1988, Proc. Natl. Acad. Sci. USA 85:5879-5883.

A “variable domain” refers to the variable region of the antibody light chain (VL) or the variable region of the antibody heavy chain (VH), either alone or in combination. As known in the art, the variable regions of the heavy and light chains each consist of four framework regions (FR) connected by three complementarily determining regions (CDRs), and contribute to the formation of the antigen-binding site of antibodies.

The “Complementarity Determining Regions” (CDRs) of exemplary agents targeting DLL3 are provided in the Sequence Table. The CDRs can be defined according to Kabat, Chothia, the accumulation of both Kabat and Chothia, AbM, contact, North, and/or conformational definitions or any method of CDR determination well known in the art. See. e.g., Kabat et al., 1991 Sequences of Proteins of Immunological Interest, 5th ed. (hypervariable regions); Chothia et al, 1989, Nature 342:877-883 (structural loop structures). AbM definition of CDRs is a compromise between Kabat and Chothia and uses Oxford Molecular's AbM antibody modeling software (Accelrys®). The identity of the amino acid residues in a particular antibody that make up a CDR can be determined using methods well known in the art.

The term “treatment” includes prophylactic and/or therapeutic treatments if it is administered prior to clinical manifestation of a condition, the treatment is considered prophylactic. Therapeutic treatment includes. e.g., ameliorating or reducing the severity of a disease, or shortening the length of the disease. Also, the term “treat,” as well as words related thereto do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In exemplary aspects, the methods treat by way of delaying the onset or recurrence of the cancer by 1 day, 2 days, 4 days, 6 days, 8 days, 10 days, 15 days, 30 days, two months, 4 months, 6 months, 1 year, 2 years, 4 years, or more. In exemplary aspects, the methods treat by way increasing the survival of the subject. In various aspects, the treatment provided by the methods of the present disclosure provides a therapeutic response as per Response Evaluation Criteria in Solid Tumors (RECIST) or other like criteria. RECIST is a set of criteria to evaluate the progression, stabilization or responsiveness of tumors and/or cancer cells jointly created by the National Cancer Institute of the United States, the National Cancer Institute of Canada Clinical Trials Group and the European Organisation for Research and Treatment of Cancer. According to RECIST, certain tumors are measured in the beginning of an evaluation (e.g., a clinical trial), in order to provide a baseline for comparison after treatment with a drug. The response assessment and evaluation criteria for tumors are published in Eisenhauer et. al., Eur J Cancer 45:228-247 (2009) and Litiére et. al., Journal of Clinical Oncology 37(13): 1102-1110 (2019) DOI: 10.1200/JCO.18.01100. In various instances, the treatment provided by the methods of the present disclosure provides a therapeutic response as per a modified RECIST tumor response assessment, as follows:

Summary of Measurement and Tumor Response
Assessment Based on Modified RECIST 1.1

| | |
|---|---|
| Measurable lesions | Non-nodal lesions: ≥10 mm (unidimensional |
| | measurement) |
| | Pathologic lymph nodes: longest diameter |
| | short axis ≥15 mm |
| Measurement of each | Non-nodal lesions: The longest diameter (mm) |
| lesion | in the axial plane |
| | Pathologic lymph nodes: short axis (mm) |
| Tumor burden | Sum of the longest diameters (SLD) of all |
| | index lesions |
| | Up to 5 lesions per organ, up to 10 total |
| Response assessment: | CR: Disappearance of all lesions |
| index lesions | Pathologic lymph nodes short axis <10 mm |
| (calculated from % | PR: ≥30% decrease from baseline |
| change in tumor | SD: Does not meet criteria for CR, PR or |
| burden) | progressive disease. |
| | Progressive disease: ≥20% increase |
| | (and ≥5 mm absolute increase) from nadir |
| Response assessment: | CR: Disappearance of all lesions |
| non-index lesions | Pathologic lymph nodes short axis <10 mm |
| | SD: Persistence of one or more non-index |
| | lesion(s) |
| | Progressive disease: Unequivocal progression |
| | of existing non-index lesions |
| New Lesions | The presence of new lesion(s) defines |
| | progression |
| Confirmation | Confirmation by subsequent assessment |
| | after ≥4 weeks required for CR, PR and |
| | progressive disease |

Summary of Modified RECIST 1.1 Overall Response Assessment

| | | | Overall Response |
|---|---|---|---|
| Index lesions (tumor | Non-Index | New | using modified |
| burden)^{a}, % | lesions | lesions | RECIST 1.1 |
| ↓ 100% | Absent | Absent | CR^{b} |
| None^{d} | Absent | Absent | CR^{b} |
| ↓ 100% | Present | Absent | PR^{b} |
| ↓ ≥30% | Absent/Present | Absent | PR^{b} |
| ↓ <30% to ↑ <20% | Absent/Present | Absent | SD |
| None^{d} | Present | Absent | SD |
| ↑ ≥20% | Any | Any | Progressive |
| | | | disease^{c} |
| Any | Unequivocal | Any | Progressive |
| | progression | | disease^{c} |
| Any | Any | Present | Progressive |
| | | | disease^{c} |
| NA/ND/UE | Absent/Present | Absent | UE |
| None^{d} | NA/ND/UE | Absent | UE |

CR = complete response;
NA = not available;
ND = not done;
PR = partial response;
RECIST = Response Evaluation Criteria in Solid Tumors;
UE = unable to evaluate
^{a}Decrease assessed relative to baseline. Increase assessed relative to nadir.
^{b}Response: CR and PR require a confirmation assessment after ≥4 weeks, may also wait until the next scheduled imaging
^{c}Progression: Progressive disease requires a confirmation assessment 4 to 6 weeks after initial radiographic progressive disease is observed
^{d}Subjects with non-index lesions only

Accordingly, methods of slowing the progression of a DLL3-positive cancer in a subject, enhancing the T cell activity or an immune response against a DLL3-positive cancer in a subject, reducing growth of a DLL3-positive tumor or DLL3-positive cancer in a subject, reducing metastasis of DLL3-positive tumor cells in a subject, increasing cell death of DLL3-positive tumor or cancer cells in a subject, delaying the onset or recurrence of a DLL3-positive cancer in a subject and/or increasing the survival of a subject are provided herein. Also, a method of treating a DLL3-positive cancer to provide a complete response (CR), partial response (PR), or stable disease (SD), as per a modified RECIST 1.1, in a subject is provided. In various aspects, the method comprises administering to the subject an anti-DLL3 agent alone, or in combination with an anti-PD-L1 antibody and/or one or more chemotherapeutic agents in accordance with the present disclosures. For example, in various aspects, the method comprises administering an anti-DLL3 agent comprising the amino acid sequence of SEQ ID NOs: 13 and 23 alone or in combination with an anti-PD-L1 antibody and/or chemotherapeutic agents.

“About” or “approximately.” when used in connection with a measurable numerical variable, refers to the indicated value of the variable and to all values of the variable that are within the experimental error of the indicated value (e.g. within the 95% confidence interval for the mean) or ±10% of the indicated value, whichever is greater. Numeric ranges are inclusive of the numbers defining the range.

“first step dose” or “run-in dose” when used in connection with administration of anti-DLL3 agents for the treatment of cancer (e.g., SCLC) refers to the initial dose of an anti-DLL3 agent in a step dose schedule or regimen. Typically, a first step dose a run-in dose equals to or is lower than a dose at which a first dose effect (e.g., cytokine release syndrome (CRS)) is observed. As known in the art, first step dose can be determined by modeling and simulation of safety and pharmacokinetic data. For example, a first step dose can be a maximum tolerated dose (QTD) of an ant-DLL3 agent where no CRS or a CRS lower than a certain grade (e.g., Grade 2) is observed.

“Target dose” when used in connection with administration of anti-DLL3 agents for the treatment of cancer (e g. SCLC) refers to a dose that achieves a target effect of an anti-DLL3 agent (e.g., ameliorating or reducing the severity of SCLC, or shortening the length of the SCLC).

“Step dose” when used in connection with administration of ant-DLL3 agents for the treatment of cancer (e.g., SCLC) refers to a dose in a step dose schedule or regimen that is higher than the previous dose at which an anti-DLL3 agent is administered. Step dose includes one or more doses that increase from a first step dose to reach a target dose.

### 2. Agents Targeting DLL3

DLL3 is a non-canonical Notch ligand expressed primarily during embryonic development that functions during somitogenesis. DLL3 accumulate in the Golgi in normal tissues (Geffers et al. J Cell Biol.178:465-476 (2007)). DLL3 was identified as a tumor-associated antigen and a compelling target for T cell-based therapies by analyzing the differential expression of this target in 28 SCLC tumors and a large panel of normal tissues (Study 123658).

The human DLL3 protein comprises several extracellular domains: signal peptide, N-terminus, DSL, EGF1, EGF2, EGF3, EGF4, EGF5, EGF6, and membrane proximal domain. The amino acid sequence of human DLL3, the EGF3 domain, the EGF4 domain, the combined EGF3 and EGF4 domains, and the membrane proximal domain are shown in the sequence table as SEQ ID NOs: 28, 29, 30, 31, and 33, respectively.

An exemplary agent targeting DLL3 is a bispecific T cell engaging antigen-binding polypeptide that binds DLL3 and CD3, such as a BiTE® molecule. BiTE® molecules are recombinant proteins made from two flexibly linked binding domains, each domain derived from antibodies. One binding domain of BITE® molecule is specific for a tumor-associated surface antigen (such as DLL3); the second binding domain is specific for CD3, a subunit of the T cell receptor complex on T cells. By their design, BiTE® molecules are uniquely suited to transiently connect T cells with target cells and, at the same time, potently activate the inherent cytolytic potential of T cells against target cells. See e.g., WO 99/54440, WO 2005/040220, and WO 2008/119567.

Accordingly, in some embodiments, the agent targeting DLL3 described comprises two binding domains: the first domain binds DLL3 (preferably human DLL3), and the second domain binds CD3 (preferably human CD3). Preferably, the first domain binds to an epitope of DLL3 comprised within the amino acid sequence of SEQ ID NO: 31. More preferably, the first domain binds to an epitope of DLL3 comprised within the amino acid sequence of SEQ ID NO: 29.

In certain embodiments, the DLL3-binding domain comprises (a) a heavy chain variable region (VH) that comprises: (i) a VH complementarity determining region one (CDR-H1) comprising the amino acid sequence of SEQ ID NO:1; (ii) a CDR-H2 comprising the amino acid sequence of SEQ ID NO:2; and (iii) a CDR-H3 comprising the amino acid sequence of SEQ ID NO:3; and (b) a light chain variable region (VL) that comprises: (i) a VL complementarity determining region one (CDR-LT) comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6.

In certain embodiments, the DLL3-binding domain comprises: a VH that comprises the amino acid sequence of SEQ ID NO:7, and a VL that comprises the amino acid sequence of SEQ ID NO:8. In certain preferred embodiments, the DLL3-binding domain comprises: a VH that comprises the amino acid sequence of SEQ ID NO:11, and a VL that comprises the amino acid sequence of SEQ ID NO:12.

In some embodiments, the VH and VL are joined by a linker to form a single chain Fv (scFv). In certain embodiments, the DLL3-binding domain comprises the amino acid sequence of SEQ ID NO: 9. In certain preferred embodiments, the DLL3-binding domain comprises the amino acid sequence of SEQ ID NO: 13.

In certain embodiments, the CD3-binding domain comprises: (a) a VH that comprises: a CDR-H1 comprising the amino acid sequence of SEQ ID NO:18, a CDR-H2 comprising the amino acid sequence of SEQ ID NO: 19, and a CDR-H3 comprising the amino acid sequence of SEQ ID NO:20; and a VL that comprises: a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO:17.

In certain embodiments, the CD3-binding domain comprises: a VH that comprises the amino acid sequence of SEQ ID NO:21, and a VL that comprises the amino acid sequence of SEQ ID NO:22. In certain embodiments, the CD3-binding domain comprises the amino acid sequence of SEQ ID NO: 23.

In certain embodiments, the anti-DLL3 agent disclosed herein comprises two domains. The first domain binds to DLL3 (preferably human DLL3) and comprises (a) a heavy chain variable region (VH) that comprises: (i) a VH complementarity determining region one (CDR-H1) comprising the amino acid sequence of SEQ ID NO: 1; (ii) a CDR-H2 comprising the amino acid sequence of SEQ ID NO:2; and (iii) a CDR-H3 comprising the amino acid sequence of SEQ ID NO:3; and (b) a light chain variable region (VL) that comprises: (i) a VL complementarity determining region one (CDR-L1) comprising the amino acid sequence of SEQ ID NO:4; (ii) a CDR-L2 comprising the amino acid sequence of SEQ ID NO:5; and (iii) a CDR-L3 comprising the amino acid sequence of SEQ ID NO:6. The second domain binds to CD3 (preferably human CD3), and comprises (a) a VH that comprises: (i) a CDR-H1 comprising the amino acid sequence of SEQ ID NO:18, (ii) a CDR-H2 comprising the amino acid sequence of SEQ ID NO:19, and (iii) a CDR-H3 comprising the amino acid sequence of SEQ ID NO: 20; and (b) a VL that comprises: (i) a CDR-L1 comprising the amino acid sequence of SEQ ID NO:15, (ii) a CDR-L2 comprising the amino acid sequence of SEQ ID NO:16, and (iii) a CDR-L3 comprising the amino acid sequence of SEQ ID NO:17.

In certain embodiments, the anti-DLL3 agent described herein comprises two domains: (a) the first domain binds DLL3 (preferably human DLL3) and comprises: a VH that comprises the amino acid sequence of SEQ ID NO:7, and a VL that comprises the amino acid sequence of SEQ ID NO: 8; and (b) the second domain binds CD3 (preferably human CD3) and comprises: a VH that comprises the amino acid sequence of SEQ ID NO:21, and a VL that comprises the amino acid sequence of SEQ ID NO:22. In certain preferred embodiments, the anti-DLL3 agent described herein comprises two domains: (a) the first domain binds DLL3 (preferably human DLL3) and comprises: a VH that comprises the amino acid sequence of SEQ ID NO:11, and a VL that comprises the amino acid sequence of SEQ ID NO: 12; and (b) the second domain binds CD3 (preferably human CD3) and comprises: a VH that comprises the amino acid sequence of SEQ ID NO:21, and a VL that comprises the amino acid sequence of SEQ ID NO:22.

In certain embodiments, the anti-DLL3 agent described herein comprises two domains: (a) the first domain binds DLL3 (preferably human DLL3) and comprises the amino acid sequence of SEQ ID NO: 9, (b) the second domain binds CD3 (preferably human CD3) and comprises the amino acid of SEQ ID NO: 23. In certain embodiments, the anti-DLL3 agent described herein comprises two domains: (a) the first domain binds DLL3 (preferably human DLL3) and comprises the amino acid sequence of SEQ ID NO: 13, (b) the second domain binds CD3 (preferably human CD3) and comprises the amino acid of SEQ ID NO: 23.

In certain embodiments, the anti-DLL3 agent described herein comprises the amino acid sequence of SEQ ID NO: 10. In certain embodiments, the anti-DLL3 agent described herein comprises the amino acid sequence of SEQ ID NO: 14.

In certain embodiments, the anti-DLL3 agent described herein comprises or consists of the amino acid sequence of SEQ ID NO: 27. In certain embodiments, the anti-DLL3 agent described herein comprises or consists of the amino acid sequence of SEQ ID NO: 33.

The anti-DLL3 agent described herein can be produced by recombinant DNA technology known in the art. For example, the anti-DLL3 agent can be produced by a process wherein a host cell (e.g., Chinese hamster ovary cells) comprising a nucleic acid encoding the anti-DLL3 agent described herein is cultured under conditions allowing the expression of the anti-DLL3 agent and the expressed anti-DLL3 agent is then recovered from the cell culture. In various embodiments, the anti-DLL3 agent is tarlatamab (International Nonproprietary Names for Pharmaceutical Substances (INN): Proposed INN: List 123, WHO Drug Information 34(2): 395-397 (2020)), also known as AMG 757. Tarlatamab is an immunoglobulin scFv-scFv-scFc, anti-[Homo sapiens DLL3 (delta-like ligand 3)] and anti-[Homo sapiens CD3E (CD3 epsilon, Leu-4)], monoclonal antibody single chain (scFv)2-scFc, bispecific; IG single chain scFv-scFv-scFc, anti-DLL3 and anti-CD3E (1-982) [scFv-VH-V-kappa anti-DLL3 (1-241) [VH (Homo sapiens IGHV4-59*01 G49>C (44) (96.9%)-(IGHD)-IGHJ4*01 (100%)) CDR-IMGT [8.7.12](26-33.51-57.96-107) (1-118)-15-mertris(tetraglycyl-seryl) linker (119-133)-V-KAPPA (Homo sapiens IGKV3-20*01 (91.7%)-IGKJ2*01 Q120>C (234) (90.9%)) CDRIMGT [7.3.9](160-166.184-186.223-231) (134-241)]-6-merseryl-tetraglycyl-seryl linker (242-247)-scFv-VH-V-lambda antiCD3E (248-496) [VH (Mus musculus IGHV10-1*02 (91.9%)-(IGHD)-IGHJ3*01 (86.7%)/Homo sapiens IGHV3-73*01 (87.0%)-(IGHD)-IGHJ5*01 (100%)) CDR-IMGT [8.10.16](273-280.298-307.346-361) (248-372)-15-mer-tris(tetraglycyl-seryl) linker (373-387)-V-LAMBDA (Homo sapiens IGLV7-43*01 (85.1%)-IGLJ3*02 (100%)) CDR-IMGT [9.3.9](413-421.439-441.478-486) (388-496)]-4-mer-tetraglycyl linker (497-500)-scFc (h-CH2-CH3)-(h-CH2-CH3) (501-982) [Homo sapiens IGHG1*03 h-CH2-CH3, nGlml (hinge 6-15 (501-510), CH2 R83>C (572), N84.4>G (577), V85>C (582) (511-620), CH3 E12 (636), M14 (638) (621-725), CHS>del) (501-725)-30-merhexakis(tetraglycyl-seryl) linker (726-755)-Homo sapiens IGHGT*03 h-CH2-CH3, nGlml (hinge 6-15 (756-765), CH2 R83>C (827), N84.4>G (832), V85>C (837) (766-875), CH3 E12 (891), M14 (893) (876-980), CHS (981-982)) (756-982)]], non-glycosylated, produced in Chinese hamster ovary (CHO) cells; immunomodulator, antineoplastic.

In certain embodiments, exemplary anti-DLL3 agents are DLL3 (e.g., human DLL3) binding molecules such as those disclosed in, e.g., WO2019234220, WO2020/069028, WO2019/131988, WO2021/200898, and WO 2021/155380, all of which are hereby incorporated by reference in their entireties. In certain embodiments, the anti-DLL3 agent is a protein that comprises (a) a first domain which is a single chain variable fragment that specifically binds to a human CD3; (b) a second domain which is a single domain antibody that specifically binds to a human serum albumin protein; and (c) a third domain which is a single domain antibody that specifically binds to a DLL3 protein. In certain embodiments, the anti-DLL3 agent comprises or consists of the amino acid sequence of SEQ ID NO: 34 or 35. In certain embodiments, the anti-DLL3 agent is a protein comprising (a) a first antigen binding domain that specifically binds to human DLL3; (b) a second antigen binding domain that specifically binds to human CD3, and (c) first and a second Fc domain, wherein the first Fc domain covalently linked to the first antigen binding domain, and the second Fc domain covalently linked to the second antigen binding domain. In certain embodiments, the first binding domain specifically binds to the membrane proximal region of the human DLL3 (e.g., SEQ ID NO: 33). In certain embodiments, the first binding domain comprises from its N- to C-terminus a first light chain variable domain, a first light chain constant domain, a first peptide linker, a first heavy chain variable domain and a first heavy chain constant CH1 domain; and the second binding domain comprises from its N- to C-terminus a second light chain variable domain, a second light chain constant domain, a second peptide linker, a second heavy chain variable domain and a second heavy chain constant CH1 domain. In certain embodiments, the anti-DLL3 agent comprises a first antigen binding domain and a second antigen binding domain, wherein the first antigen binding domain comprising or consisting of the amino acid sequence of SEQ ID NO: 36 and the second binding domain comprising or consisting of the amino acid sequence of SEQ ID NO: 37. In certain embodiments, the anti-DLL3 agent comprises a first binding domain and a second binding domain, wherein at least one of the first and second domain binds to human CD3, and a third binding domain that binds to human DLL3. In certain embodiments, one of the first or second binding domain binds to human CD3, one of the first or second binding domain binds to human CD137, and the third binding domain binds to human DLL3. In certain embodiments, the first and second binding domains are identical and bind to human CD3, and the third binding domain binds to human DLL3. In certain embodiments, the first and second binding domains are identical and bind to human CD137, and the third binding domain binds to human DLL3. Examples of such anti-DLL3 agents are disclosed in WO2021200898.

Agents targeting DLL3 disclosed herein can be used for the treatment of DLL3-positive cancer (e.g., lung cancer, small cell lung cancer) alone or in combination with other anti-cancer agents disclosed herein.

### 3. Other Anti-Cancer Agents

a. Agents Targeting PD-L1

Programmed Cell Death protein 1 (PD-1), also known as CD279, SLEB2, and hSLE1, is a transmembrane protein expressed on activated T, natural killer (NK) and B lymphocytes, macrophages, dendritic cells (DCs) and monocytes. Notably, PD-1 is highly expressed on tumor-specific T cells (Han et al., Am J Cancer Res 10(3): 727-742 (2020)). PD-1 binds to B7 protein family members, PD-1 Ligand 1 (PD-L1; also referred to as CD279 and B7-H1) and PD-1 Ligand 2 (also known as PD-L2, CD273, and B7-DC). PD-L1 is constitutively expressed on T and B cells, macrophages and dendritic cells, whereas PD-L2 expression is typically restricted to activated DC and macrophages (Xing et al., Oncoimmunology 7(3): e1356144 (2017) (doi: 10.1080/2162402X.2017.1356144)). PD-1 inhibits both adaptive and innate immune responses. The PD-1/PD-L1 axis is involved in the suppression of T cell immune responses in cancer. Antagonists of this pathway have been clinically validated across a number of solid tumor indications. PD-1 inhibitors, e.g., nivolumab, pembrolizumab, and cemiplimab, and PD-L1 inhibitors, e.g., atezolizumab, avelumab, and durvalumab, target the PD-1/PD-L1 pathway, and each has been approved by the U.S. Food and Drug Administration (FDA) for the treatment of various cancers. In various embodiments, agents targeting PD-L1 (e.g., PD-L1 blocking agent) can be used in methods disclosed herein to treat DLL3 positive cancers. Exemplary agents targeting PD-L1 include anti-PD-L1 antibodies such as atezolizumab, avelumab, and durvalumab.

In certain embodiments, the anti-PD-L1 antibody is atezolizumab (International Nonproprietary Name for Pharmaceutical Substances (INN), WHO Drug Information, Vol. 29, No. 3, 2015, Recommended INN: List 74). Atezolizumab is a humanized PD-L1 blocking antibody. It is an immunoglobulin G1-kappa, anti-[Homo sapiens CD274 (programmed death ligand 1, PDL1, PD-L1, B7 homolog 1, B7H1)], humanized monoclonal antibody; gamma1 heavy chain (1-448) [humanized VH (Homo sapiens IGHV3-23*04 (86.70%)-(IGHD)-IGHJ4*01) [8.8.11](1-118)-Homo sapiens IGHG1*03 (CH1 R120>K (215) (119-216), hinge (217-231), CH2 N84.4>A (298) (232-341), CH3 (342-446), CHS (447-448)) (119-448)], (221-214′)-disulfide with kappa light chain (1′-214′) [humanized V-KAPPA (Homo sapiens IGKV1-5*01 (87.90%)-IGKJ1*01) [6.3.9](1′-107′)-Homo sapiens IGKC*01(108′-214′)]; dimer (227-227″:230-230″)-bisdisulfide. Atezolizumab is available commercially, e.g., it is marketed as Tecentriq®.

In certain embodiments, the anti-PD-L1 antibody is avelumab (International Nonproprietary Name for Pharmaceutical Substances (INN), WHO Drug Information Vol. 30, No. 1, 2016, Recommended INN: List 75). Averlumab is a PD-L1 blocking monoclonal antibody produced in CHO cells. It is an immunoglobulin G1-lambda1, anti-[Homo sapiens CD274 (programmed death ligand 1, PDL1, PD-L1, B7 homolog 1, B7H1)], Homo sapiens monoclonal antibody; gamma1 heavy chain (1-450) [Homo sapiens VH (IGHV3-23*01 (90.80%)-(IGHD)-IGHJ4*01) [8.8.13](1-120)-IGHG1*01, Gm17,1 (CH1 (121-218), hinge (219-233), CH2(234-343), CH3 (344-448), CHS (449-450) (121-450)], (223-215′)-disulfide with lambda1 light chain (1′-216′)[Homo sapiens V-LAMBDA (IGLV2-14*01 (99.00%)-IGLJ1*01) [9.3.10](1-110′)-IGLC1*02 (111-216′)]; dimer (229-229″:232-232″)-bisdisulfide. Avelumab is commercially available, e.g., it is marketed as Bavencio®.

In certain embodiments, the anti-PD-L1 antibody is durvalumab (International Nonproprietary Name for Pharmaceutical Substances (INN), WHO Drug Information, Vol. 29, No. 3, 2015, Recommended INN: List 74). Durvalumab is a PD-L1 blocking monoclonal antibody produced in CHO cells. It is an immunoglobulin G1-kappa, anti-[Homo sapiens CD274 (programmed death ligand 1, PDL1, PD-L1, B7 homolog 1, B7H1)], Homo sapiens monoclonal antibody; gamma1 heavy chain (1-451) [Homo sapiens VH (IGHV3-7*01 (99.00%)-(IGHD)-IGHJ4*01) [8.8.14](1-121)-IGHG1*03 (CH1 (122-219), hinge (220-234), CH2 (235-344) L1.3>F (238), L1.2>E (239), P116>S (335), CH3 (345-449), CHS (450-451)) (122-451)], (224-215′)-disulfide with kappa light chain (1′-215′) [Homo sapiens V-KAPPA (IGKV3-20*01 (96.90%)-IGKJ1*01) [7.3.9](1′-108′)-IGKC*01 (109′-215′)]; dimer (230-230″:233-233″)-bisdisulfide. Durvalumab is commercially available, e.g., it is marketed as Imfinzi®.

b. Chemotherapeutic Agents

“Chemotherapeutic agents,” also referred to as antineoplastic agents, include compounds useful for the treatment of cancer. Chemotherapeutic agent can be classified according to their mechanism of action and can be further divided into subgroups within each class. Exemplary classes of chemotherapeutic agents include alkylating agents, antimetabolites, topoisomerase inhibitors, anti-tumor antibiotics, mitotic inhibitors, and protein kinase inhibitors. Alkylating agents include subgroups such as oxazaphosphorines, nitrogen mustards, imidazotetrazines, nitrosoureas, alkyl sulfonate, hydrazines, and platinum-based agents. Platinum-based agents include cisplatin, carboplatin, and oxaliplatin. Topoisomerase inhibitors include topoisomerase I inhibitors and topoisomerase II inhibitors. Mitotic inhibitors include vinca alkaloids, taxanes, and nontaxane microtubule inhibitors. Anti-tumor antibiotics include bleomycin, actinomycin D (dactinomycin), and mitomycin. Protein kinase inhibitors include BCR-ABL and c-KIT tyrosine kinase inhibitors, EGFR tyrosine kinase inhibitors, ALK tyrosine kinase inhibitors, V600E mutated-BRAF oncogene inhibitors, MEK inhibitors, Bruton kinase inhibitors. Janus kinase inhibitors, and CDK inhibitors.

In certain embodiments, the chemotherapeutic agent that can be used in the method disclosed herein is an alkylating agent. In certain embodiments, the alkylating agent is a platinum-based agent such as cisplatin, carboplatin, or oxaliplatin. In certain embodiments, the alkylating agent is lurbinectedin. Lurbinectedin is commercially available, e.g., it is marketed as Zepzelca™. In certain embodiments, the chemotherapeutic agent that can be used in the method disclosed herein is a topoisomerase inhibitor such as a topoisomerase II inhibitor (e.g., etoposide). In certain embodiments, the chemotherapeutic agent that can be used in the methods disclosed herein includes a platinum-based agent (cisplatin, carboplatin, or oxaliplatin), a topoisomerase II inhibitor (etoposide) or a combination of a platinum-based agent and a topoisomerase II inhibitor.

### 4. Dosing Regimen for Cancer Treatment Targeting DLL3

Disclosed herein are methods of treating DLL3-positive cancer (e.g., lung cancer, SCLC, neuroendocrine prostate cancer (NEPC)) comprising administering to a subject in need thereof an agent targeting DLL3, or a combination of agents targeting DLL3 and PD-L1 and/or chemotherapeutic agents. The methods comprise administering to a subject in need thereof a DLL3 targeting agent according to specific doses/regimens disclosed herein alone or in combination with a PD-L1 targeting agent and/or chemotherapeutic agents. In certain embodiments, the methods further comprise administering to the subject one or more additional therapeutic agents that prevent, reduce or mitigate the risk of adverse effects associated with the administration of the DLL3 targeting agent. In various embodiments, the DLL3 targeting agent is administered by parenteral administration. In various embodiments, the DLL3 targeting agent is administered by intravenous (IV) infusion. Unless otherwise specified herein, the DLL3 targeting agent is administered by a bolus IV infusion (e.g., an infusion over about 60 minutes). Similarly, agents targeting PD-L1 are administered by a bolus IV infusion unless otherwise specified.

a. Dosing Regimen with Agents Targeting DLL3

In one aspect, disclosed herein are methods of treating DLL3-positive cancer comprising administering to a subject in need thereof an agent targeting DLL3. Agents targeting DLL3 include the anti-DLL3 agents disclosed above. In certain embodiments, disclosed herein is a method of treating DLL3-positive cancer, comprising administering to a subject in need thereof an anti-DLL3 agent at a dose of from about 3 mg to about 100 mg twice every three weeks (21-day). In certain embodiments, the anti-DLL3 agent is administered at a dose of from about 10 mg to about 100 mg twice every three weeks. In certain embodiments, the anti-DLL3 agent is administered at a dose of about 3 mg, about 10 mg, about 30 mg, 50 mg, about 80 mg, or about 100 mg, in particular about 10 mg, about 30 mg, or about 100 mg, twice every three weeks. In certain embodiments, the anti-DLL3 agent is administered on day 1 and day 8 of a 21-day cycle.

In certain embodiments, disclosed herein is a method of treating DLL3-positive cancer, comprising administering to a subject in need thereof an anti-DLL3 agent at a dose of from about 6 mg to about 200 mg once every three weeks (21-day, Q3W). In certain embodiments, the anti-DLL3 agent is administered at a dose in the range of from about 6 mg to about 50 mg, or about 20 mg to about 100 mg, or from about 80 mg to about 150 mg, or from about 100 mg to about 200 mg once every three weeks. In certain embodiments, the anti-DLL3 agent is administered at a dose of about 6 mg, about 20 mg, about 60 mg, about 100 mg, about 180 mg or about 200 mg, in particular about 20 mg, about 60 mg, or about 200 mg, once every three weeks. In certain embodiments, the anti-DLL3 agent is administered at a dose of about 20 mg once every three weeks. In certain embodiments, the anti-DLL3 agent is administered on day 1 of a 21-day cycle.

Subjects can have an increased risk for cytokine release syndrome (CRS) during initiation of treatment with the anti-DLL3 agents (e.g., the DLL3 target agents disclosed above) due to its mechanism of action, and a step dosing approach may be implemented. Thus, in certain embodiments, the anti-DLL3 agent is administered using a step dosing approach during initiation of treatment of the agent (e.g., the first cycle of treatment) before the anti-DLL3 agent is administered according to the twice every three weeks or once every three weeks regimen described above. In such embodiments, the anti-DLL3 is administered in a 21-day cycle during initiation of the treatment (cycle 1) according to the following one step dose regimen: a first step dose or run-in dose on day 1, a step dose that equals to target dose on day 8, and a target dose on day 15. Alternatively, the anti-DLL3 agent is administered in a 21-day cycle during initiation of the treatment (cycle 1) according to the following: a first dose or run-in dose on day 1, a step dose that equal to target dose on day 8 and no anti-DLL3 agent is administered on day 15; or no anti-DLL3 agent is administered on day 1, a first dose or run-in dose on day 8, a step dose that equal to target dose on day 15.

In certain embodiments, the anti-DLL3 agent is administered in a 21-day cycle according to the following schedule: a) cycle 1: a first dose of 0 mg or 1 mg on day 1, a second dose on day 8, and a third dose on day 15, b) starting in cycle 2 of the 21-day cycle and thereafter, one or more subsequently doses administered twice every cycle, wherein each of the second, third and the one or more subsequent doses are the same and are of from about 3 mg to about 100 mg. In certain embodiments, the anti-DLL3 agent is administered in a 21-day cycle according to the following schedule: a) cycle 1: a first dose of 1 mg on day 1, a second dose on day 8, and a third dose on day 15, b) starting in cycle 2 of the 21-day cycle and thereafter, one or more subsequent doses administered twice every cycle, wherein each of the second, third, and one or more subsequent doses are the same and are of from about 10 mg to about 100 mg (e.g., 10 mg, 30 mg, or 100 mg). In certain embodiments, the anti-DLL3 agent is administered on day 1 and day 8 in cycle 2 and thereafter.

In certain embodiments, the anti-DLL3 agent is administered in a 21-day cycle according to the following schedule: a) cycle 1: a first dose of 0 mg or 1 mg on day 1, a second dose on day 8, and a third dose on day 15, b) starting in cycle 2 of the 21-day cycle and thereafter, one or more subsequent doses administered once every cycle, wherein each of the second, third and the one or more subsequent doses are the same and are from about 6 mg to about 200 mg. In certain embodiments, the anti-DLL3 agent is administered in a 21-day cycle according to the following schedule: a) cycle 1: a first dose of 1 mg on day 1, a second dose on day 8, and a third dose on day 15, b) starting in cycle 2 of the 21-day cycle and thereafter, one or more subsequent doses administered once every cycle, wherein each of the second, third, and one or more subsequent doses are the same and are of from about 20 mg to about 200 mg (e.g., 20 mg, 60 mg, or 200 mg). In certain embodiments, the anti-DLL3 agent is administered on day 1 in cycle 2 and thereafter.

In certain embodiments, the anti-DLL3 agent is administered in a 21-day cycle according to the following schedule: a) cycle 1: a first dose of 1 mg on day 1, a second dose on day 8, b) starting in cycle 2 of the 21-day cycle and thereafter, one or more subsequent doses administered once every cycle, wherein each of the second, and the one or more subsequent doses are the same and are from about 6 mg to about 100 mg (e.g., 20 mg). In such embodiments, no anti-DLL3 agent is administered on cycle 1 day 15.

In certain embodiments, the anti-DLL3 agent is administered in a 21-day cycle according to the following schedule: a) cycle 1: a first dose of 1 mg on day 8, a second dose on day 15, b) starting in cycle 2 of the 21-day cycle and thereafter, one or more subsequent doses administered once every cycle, wherein each of the second, and the one or more subsequent doses are the same and are from about 6 mg to about 100 mg (e.g., 20 mg). In such embodiments, no anti-DLL3 agent is administered on cycle 1 day 1.

In various embodiments described above, the anti-DLL3 agent is administered by parenteral administration, for example, a bolus IV infusion (e.g., an infusion over about 60 minutes). The twice every three weeks and once every three weeks dosing regimens described above can provide improved convenience and flexibility to patients and can improve the activity of the anti-DLL3 agent compared to a twice every two weeks dosing regimen.

In addition to step dosing approach, the anti-DLL3 agent can be administered via IV infusion over an extended period of time (eIV), e.g., over a period of 2 to 7 days, during initiation of treatment of the agent (e.g., cycle 1 of treatment). Compared to bolus IV administration (e.g., IV infusion over about 60 minute), eIV can achieve similar or higher cumulative serum exposures of the anti-DLL3 agent with a lower Cmax. Thus, using eIV can reduce the intensity and/or frequency of symptoms associated with CRS while achieve similar or enhanced pharmacodynamic activity (e.g., efficacy). eIV is also referred to herein as continuous intravenous infusion.

In certain embodiments, the anti-DLL3 agent is administered using an eIV approach during initiation of treatment of the agent (the first cycle of treatment) before the anti-DLL3 agent is administered according to the twice every three weeks or once every three weeks regimen described above. In such embodiments, the anti-DLL3 is administered in a 21-day cycle, wherein the anti-DLL3 agent is administered according to the following in cycle 1: starting on day 1, by continuous intravenous infusion at a dose of from about 1 mg to about 200 mg over a period of 2 days to 7 days, and optionally, on day 8, day 15, or both day 8 and day 15, by a bolus intravenous infusion at a dose of from about 10 mg to about 200 mg.

In certain embodiments, the anti-DLL3 agent is administered in a 21-day cycle according to the following regimen: a) starting on day 1 cycle 1, administering the anti-DLL3 agent at a dose of from about 1 mg to about 200 mg by continuous intravenous infusion over a period of 2 days to 7 days, and b) starting in cycle 2 and thereafter, administering the anti-DLL3 agent by a bolus intravenous infusion as follows: i) at a dose of from about 10 mg to about 100 mg twice every three weeks, or ii) at a dose of from about 20 mg to about 200 mg once every three weeks. In certain embodiments, the anti-DLL3 agent is administered at a dose of from about 10 mg to about 100 mg or from about 30 mg to about 100 mg over a period of 2 days, 3 days, 5 days, or 7 days in a). In certain embodiments, the anti-DLL3 agent is administered at a dose of from about 100 mg to about 200 mg over a period of 2 days, 3 days, 5 days, or 7 days in a). In certain embodiments, the anti-DLL3 agent is administered at a dose of 30 mg, 50 mg, or 100 mg over a period of 3 days, 5 days or 7 days in a). In certain embodiments, the anti-DLL3 agent is administered at a dose of 30 mg or 100 mg over a period of 3 days in a). In certain embodiments, the method further comprises a) administering the anti-DLL3 agent by a bolus IV infusion at a dose of from about 10 mg to about 200 mg on day 8, day 15, or day 8 and day 15 of cycle 1.

In certain embodiments, the anti-DLL3 agent is administered in a 28-day cycle according to the following regimen: a) starting on day 1 cycle 1, administering the anti-DLL3 agent at a dose of from about 1 mg to about 100 mg by continuous intravenous infusion over a period of 2 days to 7 days, and b) starting in cycle 2 and thereafter, administering the anti-DLL3 agent by a bolus intravenous infusion at a dose of from about 10 mg to about 100 mg once every two weeks. In certain embodiments, the anti-DLL3 agent is administered at a dose of from about 10 mg to about 100 mg or from about 30 mg to about 100 mg over a period of 2 days, 3 days, 5 days, or 7 days in a). In certain embodiments, the anti-DLL3 agent is administered at a dose of 30 mg, 50 mg, or 100 mg over a period of 3 days, 5 days or 7 days in a). In certain embodiments, the anti-DLL3 agent is administered at a dose of 30 mg or 100 mg over a period of 3 days in a). In certain embodiments, the method further comprises a) administering the anti-DLL3 agent by a bolus IV infusion at a dose of from about 10 mg to about 100 mg on day 8, day 15, or day 8 and day 15 of cycle 1.

The anti-DLL3 agent includes any of the DLL3 targeting agents described above. For example, the anti-DLL3 agents comprise the amino acid sequence of SEQ ID NOs: 13 and 23, or comprise or consist of the amino acid sequence of SEQ ID NO: 14, 27 or 32. In various embodiments, the anti-DLL3 agent is AMG 757. In various embodiments, the DLL3-positive cancer includes lung cancer such as SCLC or NEPC. In certain embodiments, the SCLC is relapsed/refractory SCLC (RR SCLC) or extensive disease SCLC (ED SCLC), or limited stage SCLC. In certain embodiments, the subject is a human having SCLC, e.g., RR SCLC or ED SCLC.

b. Dosing Regimen with Agents Targeting DLL3 and PD-L1

Disclosed herein are methods of treating DLL3-positive cancer comprising administering to a subject in need thereof a combination of agents targeting DLL3 and PD-L1. Agents targeting DLL3 include anti-DLL3 agents disclosed herein, agents targeting PD-L1 include anti-PD-L1 antibodies disclosed herein. In one aspect, disclosed herein is a method of treating DLL3-positive cancer comprising administering to a subject in need thereof an anti-DLL3 agent, an anti-PD-L1 antibody, and optionally one or more chemotherapeutic agents, wherein the anti-DLL3 agent is administered at a dose of from about 10 mg to about 100 mg once every two weeks. In certain embodiments, the anti-DLL3 agent is administered at a dose of about 10 mg, about 30 mg, about 50 mg, or about 100 mg once every two weeks. In certain embodiments, the anti-DLL3 agent is administered on day 1 and day 15 of a 28-day cycle.

In one aspect, disclosed herein is a method of treating DLL3-positive cancer comprising administering to a subject in need thereof an anti-DLL3 agent, an anti-PD-L1 antibody, and optionally one or more chemotherapeutic agents, wherein the anti-DLL3 agent is administered at a dose of from about 10 mg to about 100 mg twice every three weeks, or at a dose of from about 20 mg to about 200 mg once every three weeks. In certain embodiments, the anti-DLL3 agent is administered at a dose of about 10 mg, about 30 mg, about 50 mg, or about 100 mg twice every three weeks, e.g., on day 1 and day 8 of a 21-day cycle. In certain embodiments, the anti-DLL3 agent is administered at a dose of from about 20 mg to about 100 mg (e.g., about 20 mg, about 60 mg, or about 100 mg) once every three weeks, e.g., on day 1 a 21-day cycle. In certain embodiments, the anti-DLL3 agent is administered at a dose of from about 100 mg to about 200 mg (e.g., about 120 mg or about 200 mg) once every three weeks, e.g., on day 1 a 21-day cycle.

In various embodiments wherein an anti-DLL3 agent is administered together with an anti-PD-L1 antibody and optionally one or more chemotherapeutic agents, the anti-DLL3 agent can be administered according to a step dose regimen during initiation of treatment (e.g., cycle 1) to minimize potential adverse effects (e.g., CRS) associated with the anti-DLL3 agent. Thus, in certain embodiments, the anti-DLL3 agent is administered in a 21-day cycle according to the following regimen in cycle 1 of treatment: a first dose of 0 mg or about 1 mg on day 1, a second dose of from about 1 mg to about 100 mg on day 8, and a third dose of from about 10 mg to about 200 mg on day 15. In certain embodiments, the anti-DLL3 agent is administered according to the following regimen in cycle 1: a first dose of about 1 mg on day 1, a second dose of from about 10 mg to about 100 mg on day 8, and a third dose of from about 10 mg to about 100 mg on day 15. In certain embodiments, the anti-DLL3 agent is administered according to the following regimen in cycle 1: a first dose of about 1 mg on day 1, a second dose of from about 10 mg to about 100 mg on day 8, and a third dose of from about 20 mg to about 200 mg on day 15. Each of the cycle 1 regimens can be used prior to the once every two weeks, twice every three weeks, or once every three weeks regimen for the anti-DLL3 agent described above.

In certain preferred embodiments wherein an anti-DLL3 agent is administered in combination with an anti-PD-L1 agent and optional chemotherapy, the anti-DLL3 agent is administered in a 21-day cycle according to the following regimen in cycle 1 of treatment: a first dose of about 1 mg on day 1, a second dose of from about 10 mg to about 100 mg on day 8 (e.g., 20 mg), and no anti-DLL3 agent is administered on day 15. Alternatively, the anti-DLL3 agent is administered in a 21-day cycle according to the following regimen in cycle 1 of treatment: no anti-DLL3 agent is administered on day 1, a first dose of about 1 mg on day 8, a second dose of from about 10 mg to about 100 mg on day 15 (e.g., 20 mg). In such embodiments, the anti-PD-L1 agent (durvalumab or atezolizumab) is administered first followed by one or more chemotherapeutic agents and the anti-DLL3 agent if administered on the same day.

In certain embodiments, disclosed herein is a method of treating DLL3-positive cancer comprising administering to a subject in need thereof an anti-DLL3 agent, an anti-PD-L1 antibody, and optionally one or more chemotherapeutic agents, wherein the anti-DLL3 agent is administered according to the following regimen: a) cycle 1 (21 days): a first dose of about 1 mg on day one, a second dose on day 8, a third dose on day 15, b) cycles 2 and cycle 3 (21 days each cycle): a fourth dose on day 1 and a fifth dose on day 8 of each cycle, and c) one or more subsequent doses once every two weeks in a 28-day cycle starting in cycle 4 and thereafter, wherein the second, third, fourth, fifth and the one or more subsequent doses are the same and are each from about 10 mg to about 100 mg (e.g., about 10 mg, about 30 mg, or about 100 mg).

In certain embodiments, disclosed herein is a method of treating DLL3-positive cancer comprising administering to a subject in need thereof an anti-DLL3 agent, an anti-PD-L1 antibody, and optionally one or more chemotherapeutic agents, wherein the anti-DLL3 agent is administered in a 21-day cycle according to the following regimen: a) cycle 1: a first dose of about 1 mg on day one, a second dose on day 8, a third dose on day 15, b) cycles 2 and cycle 3: a fourth dose on day 1 and a fifth dose on day 8 of each cycle, and c) one or more subsequent doses once every three weeks starting in cycle 4 and thereafter, wherein the second, third, fourth, and fifth doses are the same and are each from about 10 mg to about 100 mg (e.g., about 10 mg, about 30 mg, or about 100 mg), and wherein the one or more subsequent doses are the same and are each from about 20 mg to about 200 mg (e.g., about 20 mg, about 60 mg, or about 200 mg).

In certain embodiments, disclosed herein is a method of treating DLL3-positive cancer comprising administering to a subject in need thereof an anti-DLL3 agent, an anti-PD-L1 antibody, and optionally one or more chemotherapeutic agents, wherein the anti-DLL3 agent is administered in a 21-day cycle according to the following regimen: a) cycle 1: a first dose of about 1 mg on day one, a second dose of from about 10 mg to about 100 mg on day 8, a third dose on day 15, b) cycles 2 and cycle 3: a fourth dose on day 1 of each cycle, and c) one or more subsequent doses once every three weeks starting in cycle 4 and thereafter, wherein the third, fourth, and the one or more subsequent doses are the same and are each from about 20 mg to about 200 mg (e.g., about 20 mg, about 60 mg, or about 200 mg).

In certain embodiments, disclosed herein is a method of treating DLL3-positive cancer comprising administering to a subject in need thereof an anti-DLL3 agent, an anti-PD-L1 antibody, and optionally one or more chemotherapeutic agents, wherein the anti-DLL3 agent is administered in a 21-day cycle according to the following regimen: a) cycle 1: a first dose of about 1 mg on day one, a second dose of from about 10 mg to about 100 mg on day 8 (e.g., 20 mg), b) cycles 2 and cycle 3: a third dose on day 1 of each cycle, and c) one or more subsequent doses once every three weeks starting in cycle 4 and thereafter, wherein the third and the one or more subsequent doses are the same and are each from about 10 mg to about 100 mg (e.g., about 20 mg).

In certain embodiments, disclosed herein is a method of treating DLL3-positive cancer comprising administering to a subject in need thereof an anti-DLL3 agent, an anti-PD-L1 antibody, and optionally one or more chemotherapeutic agents, wherein the anti-DLL3 agent is administered in a 21-day cycle according to the following regimen: a) cycle 1: a first dose of about 1 mg on day 8, a second dose of from about 10 mg to about 100 mg on day 15 (e.g., 20 mg), b) cycles 2 and cycle 3: a third dose on day 1 of each cycle, and c) one or more subsequent doses once every three weeks starting in cycle 4 and thereafter, wherein the third and the one or more subsequent doses are the same and are each from about 10 mg to about 100 mg (e.g., about 20 mg).

In various embodiments wherein the anti-DLL3 agent is administered together with the anti-PD-L1 antibody and optionally one or more chemotherapeutic agents, the anti-PD-L1 antibody is a PD-L1 blocking antibody. Examples of such anti-PD-L1 antibody include atezolizumab, durvalumab, and avelumab. In certain embodiments, the anti-PD-L1 antibody is atezolizumab or durvalumab. In certain embodiments, the anti-PD-L1 antibody is durvalumab. When used in methods disclosed herein, the dose and regimen of the anti-PD-L1 antibodies are the same as approved by regulatory agencies (e.g., the FDA). For example, atezolizumab can be administered at a dose of about 840 mg every 2 weeks, or about 1200 mg every 3 weeks, or about 1680 mg every 4 weeks. For example, durvalumab can be administered at a dose of about 10 mg/kg every 2 weeks, or about 1500 mg every three weeks, or about 1500 mg every four weeks.

In various embodiments wherein the anti-DLL3 agent is administered together with the anti-PD-L1 antibody and optionally one or more chemotherapeutic agents, the one or more chemotherapeutic agents include an alkylating agent, a topoisomerase inhibitor or a combination thereof. In certain embodiments, the one or more chemotherapeutic agents include a platinum-based agent (e.g., cisplatin, carboplatin, or oxaliplatin), a topoisomerase II inhibitor (e.g., etoposide) or a combination thereof. In certain embodiments, the one or more chemotherapeutic agents comprise cisplatin or carboplatin and etoposide. In certain embodiments, the one or more chemotherapeutic agents are etoposide. In various embodiments, the one or more chemotherapeutic agents are administered according to dose and/or regimen approved by a regulatory agency (e.g., the FDA). For example, in various embodiments, carboplatin is administered at a dose sufficient to achieve AUC=5 mg/ml/min, and etoposide is administered at a dose of 100 mg/m².

The anti-DLL3 agent includes any of the DLL3 targeting agents described above. For example, the anti-DLL3 agents comprise the amino acid sequence of SEQ ID NOs: 13 and 23, or comprise or consist of the amino acid sequence of SEQ ID NO: 14, 27 or 32. In various embodiments, the DLL3-positive cancer includes lung cancer such as SCLC or NEPC. In certain embodiments, the SCLC is relapsed/refractory SCLC (RR SCLC) or extensive disease SCLC (ED SCLC) or limited stage SCLC. In certain embodiments, the subject is a human having SCLC, e.g., RR SCLC or ED SCLC, or limited stage SCLC.

In various embodiments, the anti-DLL3 agent, the anti-PD-L1 antibody and the optional one or more chemotherapeutic agents are each administered by IV infusion. In certain embodiments, the anti-DLL3 agent is administered after the administration of the anti-DP-L1 antibody and the one or more chemotherapeutic agents when given on the same day.

In various embodiments disclosed herein, “combination therapy” or “in combination with” refers to administration of one treatment modality (e.g., an anti-DLL3 agent) in addition to another treatment modality (e.g., an anti-PD-L1 antibody and optionally one or more chemotherapeutic agents) to a subject (e.g., a human) having DLL3-positive cancer. In combination therapies wherein an anti-DLL3 agent and an anti-PD-L1 antibody are involved, one treatment modality can be administered before, during, or after administration of the other treatment modality to the subject. However, such combination therapy does not include situations wherein 28 or more days have elapsed between the end of administration of one treatment modality and the beginning of administration of another treatment modality.

c. Dosing Regimen with Anti-DLL3 Agents and Chemotherapeutic Agents

Disclosed herein are methods of treating DLL3-positive cancer comprising administering to a subject in need thereof a combination of anti-DLL3 agents and one or more chemotherapeutic agents. Agents targeting DLL3 include anti-DLL3 agents disclosed herein, chemotherapeutic agents include alkylating agents disclosed herein. In certain embodiments, the alkylating agent is lurbinectedin.

In certain embodiments, disclosed herein is a method of treating DLL3-positive cancer, comprising administering to a subject in need thereof an anti-DLL3 agent and an alkylating agent, wherein the anti-DLL3 agent is administered to the subject at a dose of from about 10 mg to about 200 mg (e.g., 10 mg, 20 mg, 60 mg, or 100 mg) once every two weeks. In certain embodiments, the anti-DLL3 agent is administered at a dose of from about 10 mg to about 100 mg (e.g., 10 mg, 20 mg, 60 mg, or 100 mg) twice every three weeks. In certain embodiments, the anti-DLL3 agent is administered at a dose of from about 20 mg to about 200 mg (e.g., 20 mg, 60 mg, 100 mg, or 200 mg) once every three weeks. In certain embodiments, the anti-DLL3 agent is administered on day 1 of a 21-day cycle. In various embodiments, the alkylating agent is lurbinectedin.

In certain embodiments, the method comprises administering to a subject in need thereof an anti-DLL3 agent and an alkylating agent, wherein the anti-DLL3 agent is administered to the subject in a 21-day cycle according to the following: a first dose of 0 mg or about 1 mg on day 1, a second dose of from about 10 mg to about 100 mg on day 8, a third dose of from about 10 mg to about 200 mg on day 15, and one or more subsequent doses of from about 10 mg to about 200 mg starting on day 22 and once every three weeks thereafter. In certain embodiments, the first dose is 1 mg, the second dose is from 10 mg to 100 mg (e.g., 10 mg, 20 mg, 60 mg, or 100 mg), the third dose is from 10 mg to 200 mg (e.g., 10 mg, 20 mg, 60 mg, 100 mg, or 200 mg), and the one or more subsequent doses are the same and are the same as the third dose (e.g., 10 mg, 20 mg, 60 mg, 100 mg, or 200 mg). In certain embodiments, the method comprises administering only the alkylating agent in cycle 1 and cycle 2 and administering the alkylating agent and the anti-DLL3 agent in cycle 3 and thereafter. In various embodiments, the alkylating agent is lurbinectedin. In various embodiments, lurbinectedin can be administered according to a dose and/or regimen approved by a regulatory agency (e.g., the FDA), e.g., at a dose of about 3.2 mg/m², 2.6 mg/m², or 2 mg/m² once every three weeks.

The anti-DLL3 agent includes any of the DLL3 targeting agents described above. For example, the anti-DLL3 agents comprise the amino acid sequence of SEQ ID NOs: 13 and 23, or comprise or consist of the amino acid sequence of SEQ ID NO: 14, 27 or 32

In various embodiments, the DLL3-positive cancer includes lung cancer such as SCLC. In certain embodiments, the SCLC is relapsed/refractory SCLC (RR SCLC) or extensive disease SCLC (ED SCLC). In certain embodiments, the subject is a human having SCLC, e.g., RR SCLC or ED SCLC.

In various embodiments, the anti-DLL3 agent and the alkylating agent (e.g., lurbinectedin) are each administered by IV infusion. In certain embodiments, the anti-DLL3 agent is administered after the administration of the alkylating agent when given on the same day.

In various embodiments disclosed herein, “combination therapy” or “in combination with” refers to administration of one treatment modality (e.g., an anti-DLL3 agent) in addition to another treatment modality (e.g., alkylating agents such as lurbinectedin) to a subject (e.g., a human) having DLL3-positive cancer. In combination therapies wherein an anti-DLL3 agent and lurbinectedin are involved, one treatment modality can be administered before, during, or after administration of the other treatment modality to the subject. However, such combination therapy does not include situations wherein 28 or more days have elapsed between the end of administration of one treatment modality and the beginning of administration of another treatment modality.

### 5. Additional Therapeutic Agents

In some embodiments, the methods disclosed herein further comprises the use of one or more additional therapeutic agents to prevent, reduce or mitigate the risk of adverse effects associated with the administration of the anti-DLL3 agent alone, or in combination with the anti-PD-L1 antibody and/or chemotherapeutic agents. A major adverse effect associated with the use of the anti-DLL3 agent is CRS. The one or more additional therapeutic agents useful for preventing, reducing or mitigating the risk of CRS include corticosteroids (e.g., dexamethasone), fluid (e.g., saline), and anti-IL6 antibody (e.g., tocilizumab or siltuximab). Dexamethasone may be administered by IV administration prior to all cycle 1 doses of anti-DLL3 agent (e.g., AMG 757) including all step doses, saline (e.g., 1 liter) may be administered IV following all anti-DLL3 (e.g., AMG 757) doses in cycle 1, and anti-IL6 antibody (e.g., tocilizumab or siltuximab) may be administered as needed (e.g., subject not responsive to IV fluid). Additional corticosteroid prophylaxis with oral dexamethasone may be implemented as needed. Exemplary dose of dexamethasone includes 8 mg/administration (maximum of 24 mg/day). Exemplary dose of tocilizumab includes 8 mg/kg (not to exceed 800 mg). Symptoms of CRS include fever, nausea, fatigue, headache, myalgias, malaise, and therapeutic agents useful for treating such these symptoms (e.g., paracetamol/acetaminophen for fever) may also be used. In certain embodiments, the one or more additional therapeutic agents that may also be used for reducing or mitigating adverse effects associated with anti-DLL3 agent treatment include granulocyte colony-stimulating factor (e.g., filgrastim or pegfilgrastim).

Thus, in certain embodiments, the methods disclosed herein further comprise administering one or more additional therapeutic agents selected from a corticosteroid (e.g., prednisone, hydrocortisone, and dexamethasone), a fluid (saline), and anti-IL6 antibody (e.g., tocilizumab or siltuximab). In certain embodiments, the methods further comprise administering one or more additional therapeutic agents selected from a corticosteroid (e.g., dexamethasone), a fluid (saline) and tocilizumab or siltuximab. In certain embodiments, the one or more of the corticosteroid, fluid and tocilizumab are administered in cycle 1 wherein anti-DLL3 agent (e.g., AMG 757) is administered.

In certain embodiments of any one of methods wherein one or more additional therapeutic agents are administered, the subject is a human.

### 6. Therapeutic Response

The efficacy of the disclosed methods for treating DLL3 positive cancer (such as SCLC) may be assessed by a variety of clinical outcomes, endpoints, and/or measures. In this regard, clinical outcomes that may be assessed include, but are not limited to, Progression Free Survival (PFS), Overall Survival (OS), Objective Response (ORR), Disease Control Rate (DCR), Duration of Response (DOR). The term “progression free survival (PFS),” as used herein, refers to the time from randomization until first evidence of disease progression or death. The term “overall survival (OS),” as used herein, refers to the time from randomization to death. The term “objective response rate (ORR),” as used herein, is a measure of how a specific treatment impacts tumor burden in a patient with a history of solid tumors and refers to the proportion of patients that respond either partially or fully to therapy. The term “duration of response (DoR),” as used herein, refers to the time from randomization to disease progression or death in patients who achieve complete or partial response. The term “disease control rate (DCR),” as used herein, describes the percentage of patients with advanced cancer whose therapeutic intervention has led to a complete response, partial response, or stable disease. Clinical endpoints with respect to cancer therapies are further described in, e.g., Delgado A. and Guddati, A. K., et al., Am J Cancer Res 2021; 11(4):1121-1131.

In some aspects, the disclosed methods desirably increase one or more of Progression Free Survival (PFS), Overall Survival (OS), Objective Response Rate (ORR), and/or Disease Control Rate (DCR) and Duration of Response (DOR) as compared to standard of care (SOC). For example, when the anti-DLL3 agent is used as a first-line treatment for SCLC (e.g., anti-DLL3 agent in combination with anti-PD L1 and optionally a chemotherapy agent treating a patient with no prior systemic treatment), the disclosed method desirably results in an ORR of at least about 65% (e.g., about 65% to about 70%), a median PFS greater than about 5 months (e.g., 7 months or more), and a median OS of at least about 13 months, such as about 13-15 months or about 16-18 months. When the anti-DLL3 agent is administered as a third-line treatment for SCLC (e.g., anti-DLL3 agent monotherapy treating a patient who had two or more prior therapies and relapsed), the method desirably results in an ORR of at least about 20% (e.g., about 24% or more), a median PFS greater than about 6 months, and a median OS of at least about 7.5 months (e.g., 8 months or more).

### 7. Articles of Manufacture

Disclosed herein are articles of manufacture comprising: (a) a container comprising an anti-DLL3 agent (e.g., AMG 757); and (b) a package insert with instructions for treating DLL3-positive cancer (or treating SCLC or NEPC) in a subject by administering the anti-DLL3 agent (e.g., AMG 757), wherein the instructions specify that the anti-DLL3 agent is administered at a dose of from about 10 mg to about 100 mg (e.g., 10 mg, 30 mg, or 100 mg) to the subject twice every three weeks, such as on day 1 and day 8 of a 21-day cycle. In certain embodiments, the instructions specify that the anti-DLL3 agent is administered at a dose of from about 20 mg to about 200 mg (e.g., 20 mg, 60 mg, 100 mg, 160 mg, or 200 mg) to the subject once every three weeks, such as on day 1 of a 21-day cycle. In certain embodiments, the instructions further specify that the anti-DLL3 agent is administered by extended intravenous infusion over 2 to 7 days (e.g., over 3 days) in the first cycle in which the anti-DLL3 agent is administered to the subject. In certain embodiments, the instructions further specify that the anti-DLL3 agent is administered in cycle 1 (21-day cycle) according to a one step dosing regimen: 1 mg on day 1, a second dose on day 8 and a third dose on day 15, wherein the second and third doses are the same and are each from about 10 mg to about 100 mg.

In certain embodiments, articles of manufacture comprises: (a) a container comprising an anti-DLL3 agent (e.g., AMG 757); and (b) a package insert with instructions for treating DLL3-positive cancer (or treating SCLC) in a subject by administering the anti-DLL3 agent (e.g., AMG 757) in combination with an anti-PD-L1 antibody (e.g., atezolizumab or durvalumab), wherein the instructions specify that the anti-DLL3 agent is administered at a dose of from about 20 mg to about 200 mg (e.g., 20 mg, 60 mg, 100 mg, 160 mg, or 200 mg) to the subject once every three weeks, such as on day 1 of a 21-day cycle. In certain embodiments, the instructions specify that the anti-DLL3 agent is administered at a dose of from about 10 mg to about 100 mg to the subject twice every three weeks, such as on day 1 and day 8 of a 21-day cycle. In certain embodiments, the instructions specify that the anti-DLL3 agent is administered at a dose of from about 10 mg to about 100 mg to the subject once every two weeks, such as on day 1 and day 15 of a 28-day cycle. In certain embodiments, the instructions further specify that one or more chemotherapeutic agents (e.g., carboplatin or cisplatin and/or etoposide) are administered to the subject in combination with the anti-DLL3 and anti-PD-L1 agents. In various embodiments, the package insert may specify that the anti-DLL3 agent (e.g., AMG 757) is administered in combination with chemo-immunotherapy followed by maintenance cycles of the anti-DLL3 agent plus an anti-PD-L1 agent. Alternatively, the package insert may specify that the anti-DLL3 agent (e.g., AMG 757) is administered in combination with an anti-PD-L1 agent as maintenance only therapy following standard of care chemo-immunotherapy. In embodiments wherein the anti-DLL3 agent and an anti-PD-L1 agent are used as maintenance only therapy, the package insert may further specify that subjects who have received at least 4 cycles (e.g., between 4 to 6 cycles) of platinum-based chemotherapy, etoposide, and an anti-PD-L1 agent and not have experienced disease progression are eligible, and subjects who did not have access to first line anti-PD-L1 agent and received platinum based chemotherapy and etoposide for 4 to 6 cycles are also eligible. In addition, for maintenance only therapy, the package insert may additionally specify initiating cycle 1 day 1 of the treatment within 8 weeks of the start of the last cycle of chemotherapy. The insert may further specify that prophylactic cranial radiation is permitted if radiation completes at least 7 days prior to cycle 1 day 1 wherein the anti-DLL3 agent is administered, and subject is not requiring steroids for management of central nervous system (CNS) symptoms.

In various embodiments, the package insert may further instruct hospitalizing and monitoring the subject for up to about 48 hours (e.g., about 24 hours, 12 hours, or 8 hours) after the administration of the anti-DLL3 agent (e.g., AMG 757) in cycle 1 and/or cycle 2. For example, the package insert may instruct hospitalizing and monitoring the subject for up to about 48 hours (e.g., about 24 hours, 12 hours, or 8 hours) after the first two or three doses of the anti-DLL3 agent in cycle 1. In various embodiments, the package insert may further instruct measuring or testing one or more cytokines such as IL-6, IL-8, IL-10, TNF-α, and IFN-7 of the subject (e.g., level of one or more cytokines in the subject's blood or serum) after the administration of the anti-DLL3 agent (e.g., AMG 757) in cycle 1 and hospitalizing and monitoring the subject for up to about 48 hours (e.g., about 24 hours, 12 hours, or 8 hours) if any of the level of any of the cytokines is above a normal reference level. For example, the package insert may instruct measuring or testing IL-10 of the subject after the administration of the anti-DLL3 agent (e.g., AMG 757) in cycle 1 and hospitalizing and monitoring the subject for up to about 48 hours (e.g., about 24 hours, 12 hours, or 8 hours) if the level of IL-10 is above a normal reference level. The cytokines can be measured or tested using methods known in the art.

In certain embodiments, articles of manufacture comprises: (a) a container comprising an anti-DLL3 agent (e.g., AMG 757); and (b) a package insert with instructions for treating DLL3-positive cancer (or treating SCLC) in a subject by administering the anti-DLL3 agent (e.g., AMG 757) in combination with an alkylating agent (e.g., Lurbinectedin), wherein the instructions specify, that the anti-DLL3 agent is administered at a dose of from about 20 mg to about 200 mg (e.g., 20 mg, 60 mg, 100 mg, 160 mg, or 200 mg) to the subject once every three weeks, such as on day 1 of a 21-day cycle.

In various embodiments, the container comprises an anti-DLL3 agent (e.g., AMG 757) in an amount of about 1 mg, 5 mg, 10 mg, or 25 mg, for example, the anti-DLL3 agent is supplied as a sterile, single use, preservative free lyophilized drug product containing 1, 5, 10, or 25 mg of the anti-DLL3 agent per container (e.g., vial). In various embodiments, the instructions specify that the lyophilized drug product is reconstituted with sterile water for injection. In various embodiments, the instructions specify that the subject is a human (e.g., a human having SCLC).

### 8. Subjects

In various instances of the presently disclosed methods, the subject is a human subject. In exemplary embodiments, the human subject has DLL3-positive cancers. In exemplary embodiments, the DLL3-positive cancer is small cell lung cancer (SCLC) or neuroendocrine prostate cancer (NEPC). In exemplary instances, the human subject has SCLC, optionally, a histologically or cytologically confirmed SCLC. In various aspects, the human is male or female and/or greater than or equal to 18 years of age with a SCLC. In exemplary aspects, the human subject has been treated with a platinum-based chemotherapy. In exemplary aspects, the human subject has RR SCLC, optionally, which progressed or recurred following at least one platinum-based chemotherapy with or without a PD-L1 inhibitor. In exemplary aspects, the human subject has ES-SCLC, optionally, histologically or cytologically confirmed ES SCLC. In exemplary aspects, the human subject has ES-SCLC and has received no prior systemic treatment for ES-SCLC. In exemplary instances, the human subject has an Eastern Cooperative Oncology Group (ECOG) performance status of 0-1 (Oken et al., Am J Clin Oncol 5: 649-655 (1982). In various aspects, the human subject has one or more brain metastases that have been treated. In various aspects, the human subject has liver metastases. In various aspects, the platinum-based chemotherapy comprises carboplatin or cisplatin or platinum-irinotecan. In other exemplary instances, the human subject has NEPC such as metastatic de novo or treatment-emergent NEPC. Optionally, the subject has at least 1 line of prior systemic treatment, including a platinum containing regimen for de novo NEPC (if at the time of NEPC diagnosis they had no prior diagnosis or treatment for prostate carcinoma) or an androgen signaling inhibitor (e.g., abiraterone, enzalutamide, darolutamide and/or apalutamide) if treatment-emergent (had a previous diagnosis of prostate carcinoma prior to NEPC diagnosis).

### 9. Cancer

In various aspects, the cancer treated by the presently disclosed methods is a DLL3-positive cancer (e.g., SCLC and NEPC). In various instances, the cancer treated by the presently disclosed methods is lung cancer such as a small cell lung cancer (SCLC). In exemplary aspects, the SCLC is a histologically or cytologically confirmed SCLC. Optionally, the SCLC is measurable by modified Response Criteria in Solid Tumors (RECIST) 1.1, wherein measurable lesions include (a) non-nodal lesions with clear borders that can be measured accurately and serially in one dimension in the axial plane (longest diameter≥10 mm measured by magnetic resonance imaging/computed tomography (MRI/CT) with scan slice thickness≤5 mm) and/or (b) nodal lesions with the longest diameter perpendicular to the long axis (short axis)≥15 mm on MRI/CT, and/or exclude simple cysts, pleural/pericardial effusions and ascites. In various embodiments, the cancer treated by the presently disclosed methods is neuroendocrine prostate cancer (NEPC) such as metastatic de novo or treatment-emergent NEPC. In exemplary embodiments, the NEPC is histologically diagnosed small cell NEPC, or prostate carcinoma with neuroendocrine differentiation as defined by positive immunohistochemical staining for chromogranin and/or synaptophysin in the majority of the tumor sample or 2 alterations in Tp53, RB1, and/or PTEN by immunohistochemistry (IHC) or genomic analyses of baseline tumor tissue or circulating tumor DNA (ctDNA).

### EXAMPLES

### Example 1 Clinical Experience with AMG 757

Study 20160232 is an open-label, ascending, multiple dose, phase 1 study evaluating AMG 757 in subjects with SCLC. There are two indications for this Study: A: Relapsed/refractory small cell lung cancer (RR SCLC) and B: Extensive disease SCLC (ED SCLC).

Primary Endpoints: Dose limiting toxicities (DLTs), treatment-emergent adverse events (AEs), treatment-related AEs, and clinically significant changes in vital signs, ECG, physical examinations, and clinical laboratory tests.

Secondary Endpoints: For Indications A and B: (1) PK parameters for AMG 757 following intravenous administration including but not limited to maximum observed concentration (Cₘₐₓ), minimum observed concentration (Cₘᵢₙ), area under the concentration-time curve (AUC) over the 2 week dosing interval, accumulation following multiple dosing, and, if feasible, half-life (t_{1/2}), (2) Objective Response (OR) per modified Response Evaluation Criteria in Solid Tumors (RECIST) 1.1, (3) Duration of Response (DOR), and (4) 1-year Progression-Free Survival (PFS), and (5)1-year Overall Survival (OS). For Indication B only: Relapse Free Survival (RFS).

Exploratory Endpoints: For Indications A and B: (1) Incidence of anti-AMG 757 antibody formation, (2) Changes in protein, nucleic acid and cellular biomarkers in blood (e.g., cytokines, lymphocyte status, CTCs, sDLL3), (3) Cell surface protein expression (e.g., DLL3) and tumor infiltrating lymphocyte status in tumor tissue at baseline. For Indication B only: Effect of prior chemotherapy on T cell cytokine production pre-AMG 757 treatment. Incidence of CRS (only for the part evaluating CRS mitigation strategies).

Key Eligibility Criteria for Study 20160323 are summarized in Table 3 below.

TABLE 3
Key Eligibility Criteria

| Key inclusion criteria | Key exclusion criteria |
|---|---|
| Male or female ≥18 years of age | History of other malignancy within |
| with Histologically or cytologically | the past 2 years prior to first dose |
| confirmed Small Cell Lung Cancer | of AMG 757 with exceptions |
| (SCLC) | |
| Part A: RR SCLC who progressed or | Major surgery within 28 days of first |
| recurred following platinum-based | dose AMG 757 |
| chemotherapy | |
| Part B: ED SCLC with ongoing clinical | Untreated or symptomatic brain |
| benefit (stable disease [SD], | metastases and leptomeningeal |
| partial response [PR], or complete | disease |
| response [CR]) following no more | |
| than 6 cycles of first-line platinum- | |
| based chemotherapy with the last dose | |
| of chemotherapy greater than or equal | |
| to 28 days prior to the study day 1 | |
| (first-line consolidation setting) | |
| Eastern Cooperative Oncology Group | Prior anti-cancer therapy: at least |
| (ECOG) performance status of 0-2 | 28 days must have elapsed between any |
| | prior anti- cancer therapy and first |
| | dose of AMG 757 |
| Subjects with treated brain metastases | |
| are eligible provided they meet defined | |
| criteria | |
| Adequate organ function as defined in | |
| protocol | |

AMG 757 (0.003-100.0 mg) was administered intravenously every two weeks ±step dosing in patients with SCLC that progressed after >1 platinum-based regimen. Antitumor activity was assessed using modified RECIST 1.1. Progression-free survival (PFS) and overall survival (OS) were estimated using Kaplan-Meier methods. Tumor DLL3 expression was assessed by immunohistochemistry. T-cell activation and cytokine profiles were evaluated. AMG 757 or tarlatamab administration continued until disease progression, unacceptable side effects, or consent withdrawal.

The analysis included patients enrolled in the escalation and expansion cohorts. The data cutoff was Jul. 19, 2022. A two-parameter Bayesian Logistic Regression Model (BLRM) model guided dose exploration. Safety data was reviewed on an ongoing basis. In dose level review meetings (DLRMs), the Sponsor, in consultation with site investigators, reviewed the BLRM recommended dose level and all available cumulative data by cohort prior to making dose escalation decisions. AEs and DLTs observed in all subjects were evaluated continually and fully integrated into all DLRMs. Based on the overall benefit-risk profile of 100 mg, it was decided to further evaluate this as the expansion dose. Descriptive statistics are provided for selected demographics, safety, pharmacokinetics (PK), pharmacodynamics, and biomarker data. Kaplan-Meier methods were used to estimate the median and percentiles for time to event endpoints with confidence interval (CI) calculated using the Brookmeyer and Crowley method.

Maximum tolerated dose (MTD) is the highest dose deemed safe as determined collaboratively by investigators and study team with consideration of the Bayesian Logistic Regression Model (BLRM).

Adverse events were graded using Common Terminology Criteria for Adverse Events (CTCAE), version 4.0. CRS events were graded using the Lee criteria.

Additionally, cytokine release syndrome (CRS), neutropenia, and neurologic events were monitored as events of interest in this study using an Amgen MedDRA Query narrow (AMQN) search approach. All events were coded using MedDRA version 24.1. Cytokine release syndrome by AMQN search includes Cytokine abnormal, Cytokine release syndrome, Cytokine storm, and Cytokine test. CRS events were graded using CRS Lee et al. (2014) criteria. Neutropenia was based on AMQN search and graded using CTCAE version 4.0. Neurologic Events were based on “Central neuropsychiatric events due to direct neurotoxicities” AMQN search and graded using CTCAE version 4.0.

Efficacy data disclosed herein were based on local investigators evaluations. Patients were defined as evaluable for efficacy if data cut-off date was at least 9 weeks after the first dose date to allow time for assessment.

Exploratory analyses of T cell and peripheral cytokines was performed on serially collected blood samples.

For immunogenicity assessments, blood samples from patients who received tarlatamab were collected on study day 1 (pre-dose) and multiple timepoints during the study for detection of anti-tarlatamab binding antibodies using a validated electro-chemiluminescent bridging immunoassay.

### Immune Cell and Cytokine Analysis

Whole blood samples drawn into EDTA tubes were collected according to the schedule of assessment specified in the study protocol. A flow cytometry validated panel was used to stain whole blood samples using fluorescently labeled antibodies CD4 BV510 (clone SK3, BD Biosciences), CD8 BV605 (clone SKI, BD Biosciences), CD3 Alexa Fluor 700 (clone SK7, BioLegend), and CD279 (PD-1) BB515 (clone EH12.1, BD Biosciences). Data were acquired centrally at Q2 Solutions Laboratories Europe on a BD FACSCanto flow cytometer. To evaluate cytokine production, serum samples were collected and IFNγ levels were assessed using the Meso Scale Discovery (MSD) V-plex Pro-inflammatory Panel 1. The assay was performed according to the manufacturer's instructions. In brief, samples were diluted 1:2 with Diluent 2 (MSD). Diluted samples and standards were added in duplicate to a 96-well plate with capture antibodies independently precoated on 10 defined spots and incubated for 2 hours at ambient temperature. The plates were washed three times with wash buffer and a detection antibody mixture was added to each well and the plate was incubated for 2 hours at ambient temperature. The plate was washed three times with wash buffer and 2× Read Buffer T (MSD) was added to each well and read on an MSD plate reader. The concentrations were extrapolated from the standard curve within the established range of 2.61-542,720 pg/mL.

### Characterization of Cytokine Release Syndrome (CRS)

An analysis was performed in order to explore correlations between cytokine levels within 24 hours following the initial dose of tarlatamab and occurrence of CRS in Cycle 1. Cohorts receiving 1 mg as an initial dose of tarlatamab and from 1-100 mg in subsequent doses in Cycle 1 were included in this analysis.

Serum was drawn at time points up to 24 hours for cytokine analysis. The incidence, time to onset, severity, management, and recurrence of CRS were assessed. Serum peak level and elevation speed within 24 hours following the initial dose of tarlatamab were evaluated for a panel of soluble factors in patients with CRS versus no CRS in Cycle 1. Patients from 1 mg initial dosing cohorts were included.

A Kruskal Wallis (KW) rank-based test with false discovery rate correction was used to identify whether analyte values from the CRS and no CRS categories originate from different distributions. A Jonckheere-Terpstra (JT) trend test was utilized along with false discovery rate correction to identify increasing trend in analyte values from the no CRS to the CRS bucket. Univariate logistic regression was utilized to determine whether the growth rate and peak of each analyte was predictive of the occurrence of CRS in Cycle 1 post dosing.

### Results

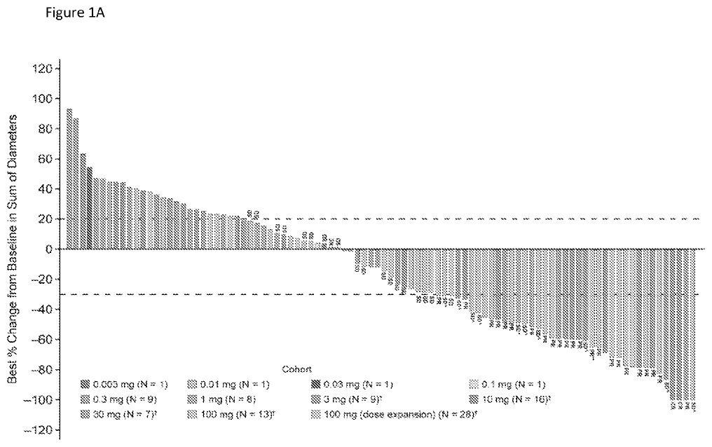

As of Jul. 19, 2022, 107 patients received tarlatamab in dose escalation (0.003-100 mg; n=73) and expansion (100 mg; n=34) cohorts (FIG. 1). Step-dosing was utilized starting with the 3 mg cohort (using 1 mg as the run-in dose followed by target dose on day 8, day 15, and Q2W thereafter) due to observed cytokine release syndrome (CRS) in prior cohorts. Cytokine release syndrome (CRS) is an expected risk of tarlatamab per its mechanism of action (MOA).

Baseline characteristics are summarized in Table 3. Median age was 63 years (range, 32-80). ECOG performance status was 0-1 in 99% of patients. More than 70% of patients had ≥2 lines of prior therapy, 25% were platinum refractory and 50% had prior PD-1/PD-L1 inhibitor.

The median follow-up was 8.7 months (range, 0.2-31.8). Treatment was discontinued in 92 patients (86%) most commonly for disease progression (n=77 [72%]). At data cutoff, 47 patients (43.9%) had ended study due to death. Median number of treatment cycles started was 3 (interquartile range [IQR]: 1, 8) and median number of tarlatamab doses received was 6 (IQR: 3, 16).

TABLE 3
Patient Demographics and Baseline Characteristics

All patients
(N = 107)

Age, years

| | |
|---|---|
| Median (IQR) | 63.0 (58.0, 69.0) |

Sex, n (%)

| | |
|---|---|
| Male | 61 (57%) |
| Female | 46 (43%) |

Race, n (%)

| | |
|---|---|
| White | 86 (80%) |
| Asian | 13 (12%) |
| Black/African American | 3 (3%) |
| Other | 5 (5%) |

Ethnicity, n (%)

| | |
|---|---|
| Hispanic/Latino | 2 (2%) |
| Not Hispanic/Latino | 105 (98%) |

Smoking history, n (%)

| | |
|---|---|
| Never | 10 (9%) |
| Current | 14 (13%) |
| Former | 81 (76%) |
| Missing | 2 (2%) |

ECOG performance status, n (%)

| | |
|---|---|
| 0 | 40 (37%) |
| 1 | 66 (62%) |
| 2 | 1 (1%) |

Prior lines of therapy

| | |
|---|---|
| Median (IQR) | 2.0 (1.0, 3.0) |
| 1, n (%) | 30 (28%) |
| 2, n (%) | 45 (42%) |
| ≥3, n (%) | 32 (30%) |

Most recent line platinum-treated patients, n (%)

| | |
|---|---|
| Platinum-sensitive | 54 (51%) |
| Platinum-resistant | 22 (21%) |
| Platinum-refractory | 26 (25%) |
| Not Assessable/missing | 4 (4%) |

Prior radiotherapy, n (%)

| | |
|---|---|
| Yes | 85 (79%) |
| No | 22 (21%) |

Prior anti-PD-1 or anti-PD-L1, n (%)

| | |
|---|---|
| Yes | 53 (50%) |
| No | 54 (51%) |

Metastatic at baseline, n (%)

| | |
|---|---|
| Yes | 100 (94%) |
| Brain metastases | 27 (25%) |
| Liver metastases | 54 (51%) |
| No | 7 (7%) |

History of treated brain metastases

| | |
|---|---|
| Yes | 39 (36%) |
| No | 68 (64%) |
| Extensive stage disease, n (%)* | 100 (94%) |

Sum of diameters of target lesions at baseline, mm

| | |
|---|---|
| Median (IQR) | 75.0 (43.0, 108.0) |

*Stage of disease unknown for 1 patient at baseline.
ECOG, Eastern Cooperative Oncology Group, IQR, interquartile range; PD-1, programmed cell death protein 1; PD-L1, programmed cell death ligand 1.

Retrospective DLL3 immunohistochemistry analysis was performed on fresh or archived biopsy as described above. DLL3 was expressed (≥1%) in 85 of 90 (94%) evaluable patients; median H-score was 186 (range, 0-300) and median tumor cell positivity was 95% (range, 0-100%).

### Safety and Tolerability

DLTs occurred in 6 patients including pneumonitis (n=1 [last prior dose, 0.3 mg]), increased alanine aminotransferase (n=1 [1 mg], CRS (n=1 [1 mg]), encephalopathy (n=1 [10 mg]), chills, pyrexia, and neutropenia (n=1 each [100 mg]). A maximum tolerated dose (MTD) was not reached; the highest dose (100 mg) was evaluated in the expansion cohort. Four patients (3.7%) discontinued tarlatamab due to AEs of encephalopathy (n=1), immune effector cell-associated neurotoxicity (ICANS) (n=1), and pneumonitis (n=2), all of which were treatment related. A single G5 pneumonitis event was recorded in a 70-year old male with a history of prior carboplatin/etoposide chemotherapy, chronic obstructive pulmonary disease, and radiation to the lung and pleural nodules. The event onset was cycle 1 day 18, 3 days after the second tarlatamab treatment (both doses 0.3 mg) and was confounded by clinically significant disease progression at the time of pneumonitis requiring urgent palliative radiation to the lung and to a soft tissue mass in the thoracic spine causing spinal cord compression. The cause of death was attributed by the investigator to disease progression and pneumonitis. An additional G3 and three additional G2 TEAEs of pneumonitis were observed (5/107 [4.7%] overall incidence of pneumonitis). Among patients with G2 pneumonitis, 1 patient ended treatment due to neurotoxicity (not pneumonitis), 1 patient had resolution of pneumonitis prior to discontinuation for PD, and 1 patient resumed treatment without dose change.

TEAEs of any cause/grade occurred in 107 patients (100%). The most common were CRS (56 patients [52.3%]), pyrexia (43 [40.2%]), constipation (33 [30.8%]) and fatigue (32 [29.9%]). Grade≥3 AEs occurred in 61 patients (57.0%) with the most common being neutropenia (8.4%), decreased lymphocyte count (6.5%), and hypertension (5.6%). Serious adverse events (SAEs) occurred in 55 patients (51.4%). TEAEs led to dose reductions in 9 patients (8.4%) with 4 (3.7%) having CRS-related reductions. Dose interruption occurred in 20 patients (18.7%), most commonly for neutropenia and decreased neutrophil count. Any grade and grade≥3 TRAEs occurred in 97 (90.7%) and 33 (30.8%) patients, respectively.

CRS, neutropenia, and neurologic events were monitored as events of interest based on preclinical, clinical, and mechanistic data with tarlatamab, other BiTE™ molecules, and other T cell-associated therapies. Amgen MedDRA Query narrow (AMQN) searches were performed to supplement standard system organ class single preferred term safety reporting (defined above and summarized in Table 4). Measures to ameliorate the potential for CRS included prophylactic corticosteroids (cycle 1 only) and IV hydration in some patients. Grade>2 treatment-emergent CRS was reported in 15 patients (14.0%) and grade 3 CRS in 1 patient (0.9%); no grade 4 or 5 CRS has been reported. For any grade CRS (n=56), median time to first onset was 2 days (range, 1-30 days) after first dose based on recorded date; more precise time-based reporting was implemented to better characterize CRS, with median time to onset of 17.5 hours in the subset of patients with hourly data available (n=47). CRS was transient (median duration, 3 days [IQR: 2-4 days]) and resolved in all cases. Eight patients (7.5%) received tocilizumab for CRS. CRS was largely confined to cycle 1. A total of 5 patients (4.7%) had CRS in cycle 2; 4 of these patients also had CRS in cycle 1, while one patient experienced CRS for the first time in cycle 2 or later. Treatment-emergent neurologic AEs of any grade occurred in 75 patients (70.1%) and were mostly grade 1; dysgeusia (29.0%), headache (19.6%), and dizziness (10.3%) were the most common. Grade≥3 treatment-emergent neurologic events occurred in 12 patients (11.2%) including confusional state (4.7%), delirium (1.9%), and encephalopathy (1.9%). One subject had a grade 4 neurologic event (confusion), none had grade 5. All grade≥3 neurologic AEs resolved, with 1 subject discontinuing tarlatamab due to G3 encephalopathy and 2 other subjects continuing treatment at reduced doses. G2 ICANS was the other neurologic cause leading to discontinuation in 1 subject. First onset of any grade neurological event was mostly within the first 30 days of treatment (median, 9 days [IQR, 2-29 days] with a median duration 5 days (IQR, 2-15 days). Grade≥3 neutropenia occurred in 11 patients (10.3%). Any grade neutropenia first onset occurred at a median of 30 days (IQR, 21-31 days) after first tarlatamab administration, and median duration was 7 days (IQR, 4-13); overall, 10 patients (9.3%) received G-CSF. Febrile neutropenia occurred in 1 patient and was not considered treatment related.

TABLE 4
Treatment-emergent Adverse Events (Preferred term and AMQ for selected terms)

| All patients | | | | |
|---|---|---|---|---|
| (N = 107) | | | | |
| Any grade | Grade ≥2 | Grade ≥3 | Grade ≥4 | Grade 5 |

Adverse events of any cause that
occurred during treatment*

| | | | | | |
|---|---|---|---|---|---|
| Any | 107 (100%) | 94 (88%) | 61 (57%) | 13 (12%) | 1 (1%) |
| Serious | 55 (51%) | 44 (41%) | 30 (28%) | 7 (7%) | 1 (1%) |
| Resulting in | 4 (4%) | 4 (4%) | 3 (3%) | 0 (0.0) | 0 (0.0) |
| discontinuation | | | | | |

Adverse events of any cause that occurred during treatment
(any grade in >10% of patients or grade ≥3 in >5%)*

| | | | | | |
|---|---|---|---|---|---|
| Cytokine release | 56 (52%) | 15 (14%) | 1 (1%) | 0 | 0 |
| syndrome | | | | | |
| Pyrexia | 43 (40%) | 11 (10%) | 2 (2%) | 0 | 0 |
| Constipation | 33 (31%) | 8 (8%) | 0 | 0 | 0 |
| Fatigue | 32 (30%) | 15 (14%) | 4 (4%) | 0 | 0 |
| Nausea | 31 (29%) | 6 (6%) | 1 (1%) | 0 | 0 |
| Dysgeusia | 31 (29%) | 6 (6%) | 0) | 0 | 0 |
| Decreased appetite | 29 (27%) | 15 (14%) | 0 | 0 | 0 |
| Anemia | 22 (21%) | 17 (16%) | 4 (4%) | 0 | 0 |
| Headache | 21 (20%) | 1 (1%) | 0 | 0 | 0 |
| Weight decreased | 19 (18%) | 8 (8%) | 1 (1%) | 0 | 0 |
| Dyspnea | 18 (17%) | 8 (8%) | 4 (4%) | 0 | 0 |
| Asthenia | 17 (16%) | 11 (10%) | 2 (2%) | 0 | 0 |
| Vomiting | 16 (15%) | 3 (3%) | 1 (1%) | 0 | 0 |
| Arthralgia | 15 (14%) | 2 (2%) | 0 | 0 | 0 |
| Back pain | 14 (13%) | 7 (7%) | 0 | 0 | 0 |
| Hypotension | 14 (13%) | 7 (7%) | 0 | 0 | 0 |
| Cough | 14 (13%) | 2 (2%) | 0 | 0 | 0 |
| Hypertension | 13 (12%) | 9 (8%) | 6 (6%) | 0 | 0 |
| Neutropenia | 12 (11%) | 11 (10%) | 9 (8%) | 3 (3%) | 0 |
| Hyponatremia | 12 (11%) | 7 (7%) | 5 (5%) | 0 | 0 |
| White blood cell | 11 (10%) | 9 (8%) | 5 (5%) | 1 (1%) | 0 |
| count decreased | | | | | |
| Diarrhea | 11 (10%) | 3 (3%) | 1 (1%) | 0 | 0 |
| Dizziness | 11 (10%) | 2 (2%) | 0 | 0 | 0 |
| Lymphocyte count | 10 (9%) | 8 (8%) | 7 (7%) | 3 (3%) | 0 |
| decreased | | | | | |

Adverse events of interest of any cause^{†}

| | | | | | |
|---|---|---|---|---|---|
| Neurologic events | 75 (70%) | 29 (27%) | 12 (11%) | 1 (1%) | 0 |
| CRS | 56 (52%) | 15 (14%) | 1 (1%) | 0 | 0 |
| Neutropenia | 17 (16%) | 16 (15%) | 11 (10%) | 4 (4%) | 0 |

*Based on single preferred term incidence. Coded using MedDRA version: 25.0. Adverse events graded using CTCAE version 4.0 and CRS events graded using Lee et al. (2014) criteria.
^{†}CRS based on AMQ narrow search, which includes Cytokine abnormal, Cytokine release syndrome, Cytokine storm, Cytokine test. Neutropenia based on AMQ narrow search. Neurologic Events based on “Central neuropsychiatric events due to direct neurotoxicities” AMQ narrow search. Coded using MedDRA version: 25.0. CRS events graded using CRS Lee et al. (2014) criteria. Neutropenia and neurologic events graded using CTCAE version 4.0.
AMQ, Amgen MedDRA Query.

### Efficacy

Confirmed ORR was 23.4% (95% confidence interval [CI]: 15.7, 32.5) including 2 complete and 23 partial responses (Table 5).

TABLE 5
Tumor Response to Tarlatamab According
to Investigator Assessment

| | |
|---|---|
| | Interim efficacy analysis set* |
| Response | (N = 107) |

Objective response rate, % (95% CI)

| | |
|---|---|
| Confirmed | 23% (15.7-32.5) |
| Confirmed and unconfirmed | 25% (17.3-34.6) |
| Disease control rate, % (95% Cl) | 51% (41.5-61.2) |

Best overall response, n (%)

| | |
|---|---|
| Confirmed complete response | 2 (2%) |
| Confirmed partial response | 23 (22%) |
| Stable disease | 30 (28%) |
| Progressive disease | 9 (8%) |
| Could not be evaluated^{†} | 34 (32%) |
| No assessment^{‡} | 9 (8%) |
| Median time to response (IQR), months | 1.81 (1.68-1.91) |
| Median duration of objective response | 12.3 (6.6-14.9) |
| (95% CI), months | |

*The Interim efficacy analysis set is a subset of the safety analysis set. The Interim efficacy analysis set include patients whose data cut-off date is at least 9 weeks after the first dose date.
^{†}This includes 32 patients who had PD in the post-baseline scan however without further confirmation scan (unconfirmed PD per modified RECIST 1.1).
^{‡}Reasons for no imaging assessment included consent withdrawn (n = 5), death (n = 2), clinical PD (n = 1), and starting new anticancer therapy (n = 1).

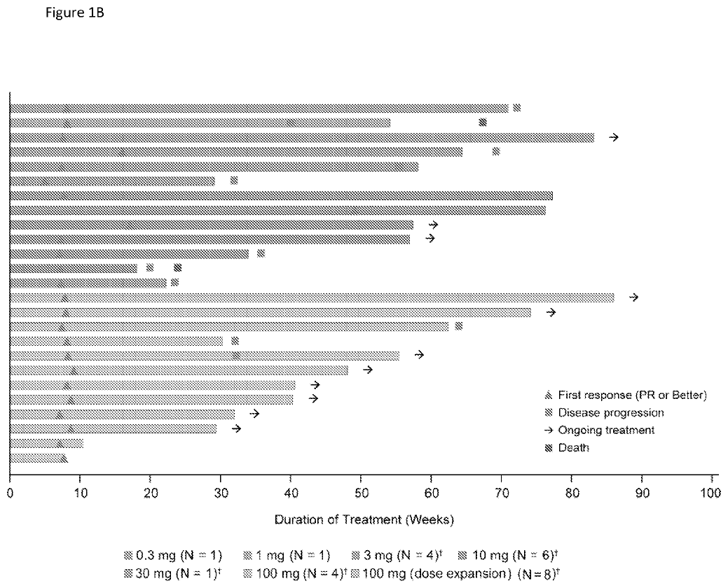

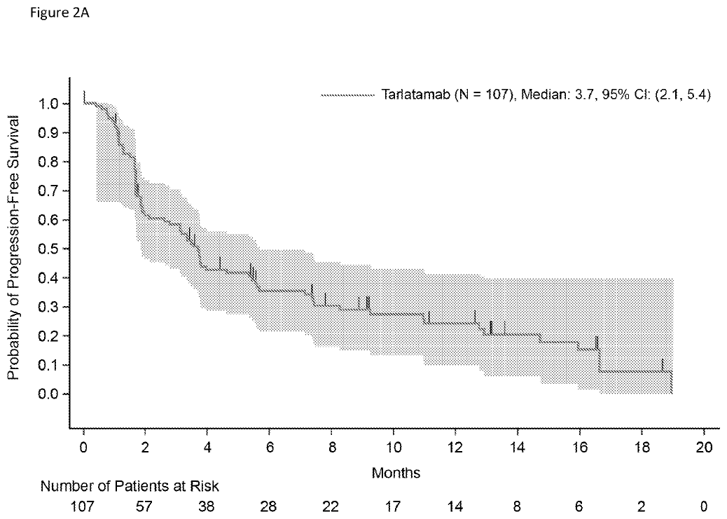

FIG. 1A illustrates best percentage change from baseline in sum of diameters for patients with evaluable postbaseline assessments (n=94). Disease control rate was 51.4% (95% CI: 41.5, 61.2). Responses were seen starting with the 0.3 mg dose and generally higher rates of response were observed at doses of 3 mg and above. At least 30% tumor shrinkage in target lesions at post-baseline assessment was observed in 39 patients (36.4%). Among confirmed responders, median TTR was 1.8 months (range, 1.2-7.4) and median DOR was 12.3 months (95% CI: 6.6, 14.9) (FIG. 1B). Longest duration of response was 14.9 months and 11 patients (44% of responders) had ongoing response at data cutoff. Median PFS was 3.7 months (95% CI: 2.1, 5.4) and median OS was 13.2 months (95% CI: 10.5, NE) (FIG. 2), respectively. A total of 28 patients (26.2%) went on to subsequent anticancer therapies after tarlatamab.

### Clinical Pharmacokinetics

As of Apr. 15, 2022, preliminary pharmacokinetic data from dose escalation and expansion cohorts were available for 101 patients. Briefly, tarlatamab exhibited approximate dose proportional increase in serum exposures. Approximate steady state in serum tarlatamab exposures were achieved within 4 weeks of every-other-week target regimen initiation, with minimal accumulation. The mean (±SD) terminal elimination half-life estimated at steady-state across the evaluated target dose range was approximately 5.7 (±2.2) days, which is consistent with the intended half-life extension of the HLE platform relative to non-HLE BiTE™ molecules.

### Immunogenicity

Among the patients with available samples, 10 of 97 (10.3%) developed anti-tarlatamab antibodies after tarlatamab administration. Two of 99 (2.0%) patients had pre-existing antibodies at baseline. There was no apparent anti-drug antibody (ADA) impact on tarlatamab exposures or on the safety profile in these patients.

### Pharmacodynamics

The pharmacodynamic response after the first dose of tarlatamab infusion was characterized by initial T cell redistribution, T cell activation, and transient IFN-gamma elevation. For step-dose cohorts, pharmacodynamic responses were greatest after initial administration of 1 mg step dose and were not exceeded with target dose administration.

### Clinical CRS Summary

CRS was mostly grade 1 (39%), occurred in Cycle 1, and was reversible in all patients (see Table 6). CRS was clinically manageable.

TABLE 6

| | All Patients |
|---|---|
| Clinical CRS* Summary | (N = 106) |
| CRS, n (%) | 56 (53) |
| Grade 1 | 41 (39) |
| Grade 2 | 14 (13) |
| Grade 3 | 1 (1) |
| Occurred in Cycle 1^{†} | 55 (98) |
| Occurred in Cycle ≥2^{†} | 5 (9) |
| Received oxygen for CRS^{†} | 3 (5) |
| Intravenous fluid use for hypotension in CRS^{†} | 8 (14) |
| Vasopressor use for hypotension in CRS^{†} | 2 (4) |
| Received tocilizumab for CRS^{†} | 8 (14) |
| Resolution^{†} | 56 (100) |
| Median (range) time of CRS onset, h^{‡} | 17.5 (3, 683) |
| Median (range) duration of CRS, d | 3 (1, 22) |

To mitigate the risk of CRS, ≥1 of the following prophylactic measures were able to be used during cycle 1: additional corticosteroid prophylaxis with oral dexamethasone, administration of tocilizumab, etanercept, or acetaminophen.
*CRS includes cytokine abnormal, cytokine release syndrome, cytokine storm, cytokine test.
^{†}Percentage based on the total number of patients with any grade CRS.
^{‡}Time of onset in patients with both date and hour of onset data (n = 47).
Data cutoff: Jun. 15, 2022.
CRS, cytokine release syndrome.

### Cytokines and CRS Analysis

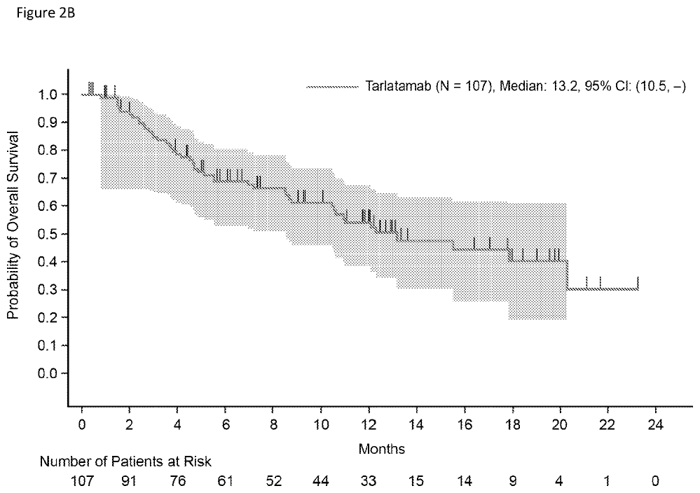

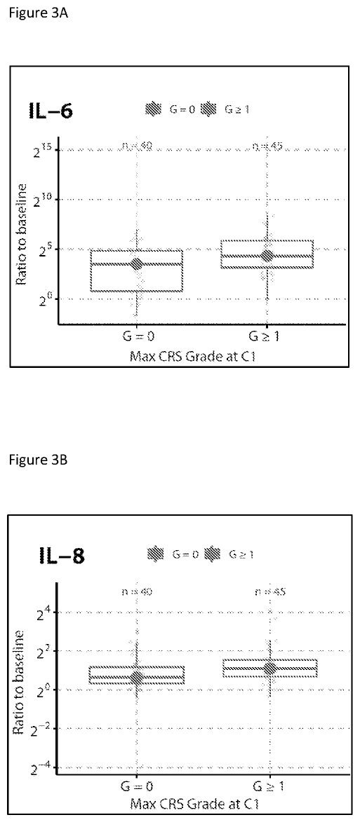

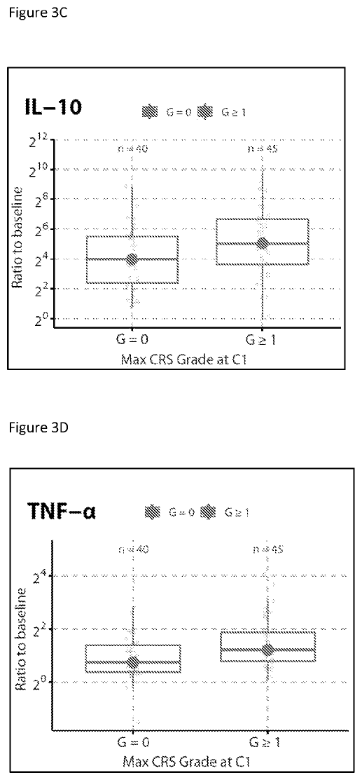

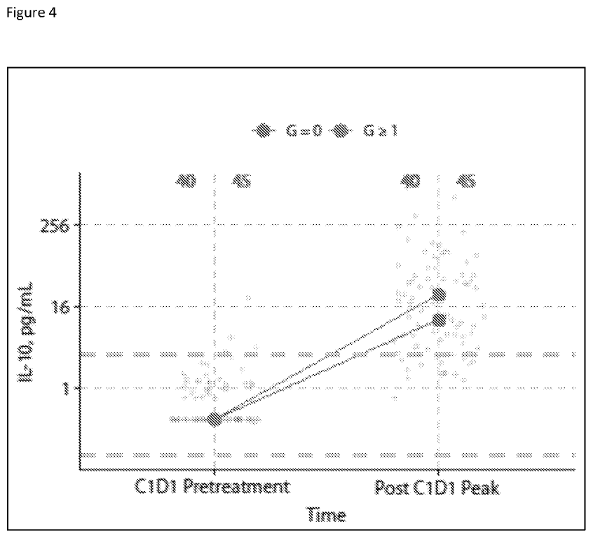

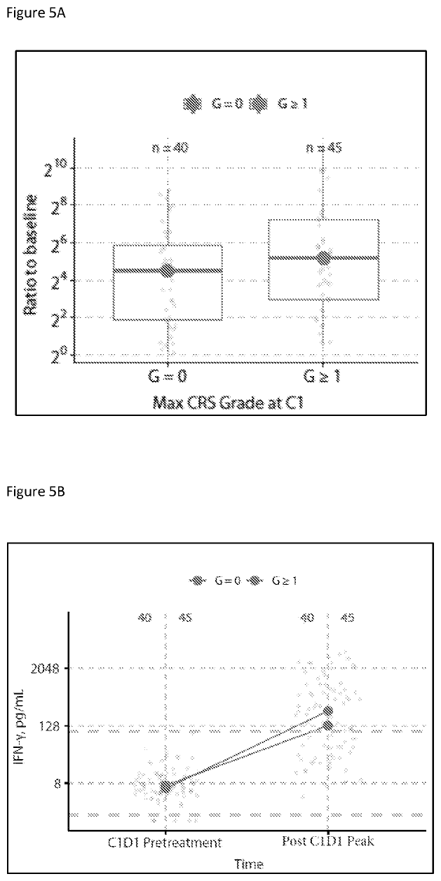

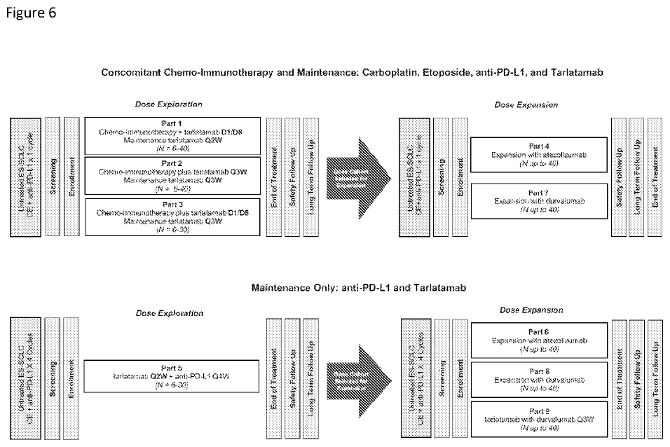

In biomarker evaluable patients, the ratio of peak level within 24 hours to baseline level for IL-6, IL-8, IL-10, and TNF-α trended higher in the patients with CRS in Cycle 1 versus patients without (FIGS. 3A-3D). IL-10 showed significant elevation above referenced normal range, and was higher in patients with CRS (FIG. 4). The effector cytokine IFN-γ was investigated due to its strong induction in preclinical experiments (see, e.g., Giffin M J, et al. Clin Cancer Res. 2021; 27:1526-1537). As expected from tarlatamab's mechanism of action, IFN-γ induction was above the physiologic range; induction was similar between those with Cycle 1 CRS versus those without Cycle 1 CRS (FIGS. 5A and 5B).

### Discussion

Tarlatamab demonstrated a manageable safety profile across a wide dose range through the expansion dose of 100 mg and was associated with encouraging response rates in a heavily pretreated population of SCLC patients. Confirmed responses were durable and OS appeared promising. Across all doses (N=107), tarlatamab was discontinued in only 4 patients (3.7%) and dose reductions were implemented for 9 patients due to AEs. An MTD was not reached; the highest dose (100 mg) was further evaluated in the dose expansion cohort.

CRS was expected based on the MOA of tarlatamab. While CRS was the most frequent TEAE observed in this study (56% of patients), it was generally low-grade, transient, and typically occurred in the first cycle. CRS was typically reversible and managed with steroids, IV fluids, and anti-pyretics, with tocilizumab used to treat CRS in 8 out of 107 patients receiving tarlatamab (7.5%). Neutropenia was a risk associated with tarlatamab observed in this study and was unexpected based on pre-clinical data; the mechanism is not understood. The study protocol was updated accordingly for specific monitoring and management. Further evaluation of neutropenia will be relevant to trials of tarlatamab use in combination with other marrow suppressing therapies. Neurologic evaluation was conducted as part of frequent clinical evaluation to assess study patients for CRS and/or neurologic AEs due to the known association with immune-effector cell therapies. Most neurologic AEs were mild and self-limiting without the need for treatment discontinuation or dose reduction, though there were 12 patients (11.2%) with grade≥3 neurologic AEs. There were 2 patients who discontinued tarlatamab due to neurologic AEs (encephalopathy, ICANS). Careful evaluation of neurologic AEs is ongoing to better characterize these events and identify risk factors or interventions that might specifically improve management.

There are few approved therapies for SCLC after first line. A phase 2 study of lurbinectedin in second line SCLC found an ORR of 35% and median DOR of 5.3 months. In a randomized study of topotecan vs combination chemotherapy in recurrent SCLC, topotecan ORR was 24% and median DOR was 3.3 months. The prior conditional approval by US FDA of nivolumab and pembrolizumab for third line or later SCLC was based on response rates of 12% and 19%, respectively, with durable responses seen at ≥12 months in >60% of responding patients. These approvals were subsequently withdrawn as survival benefit was not demonstrated. The ORR of 23% and median DOR of 12.3 months for tarlatamab compares well with other therapies, especially considering over 70% of patients had at least 2 prior lines of therapy. Half of the patients in this study (50%) had received prior PD-1/PD-L1 therapy, representative of current practice in first line SCLC. Despite the median PFS (3.7 months) seen with tarlatamab, the median OS (13.2 months) is relatively high and compares favorably with 9.3-month median OS reported previously with lurbinectedin or about 6-month OS with topotecan, though the value of comparisons is limited by differences in study design and patient populations. The promising OS benefit may reflect the long durability of response seen thus far in those who respond to tarlatamab, but further follow up is needed in larger randomized studies. An alternative explanation of the relatively long OS with a short PFS could be that OS benefit derived from post-tarlatamab treatment, though this is less likely a major factor because only 26.2% of patients received such treatment in this heavily pretreated cohort. Identifying clinical, demographic, and biological factors predictive of response and/or toxicity (e.g., prior therapies, DLL3 expression) is an ongoing effort. Increased DLL3 expression appears to trend with a higher magnitude of clinical benefit.

The results of this example demonstrate promising activity of tarlatamab in patients with a high unmet medical need, and have led to several ongoing investigations of tarlatamab as monotherapy in SCLC and other neuroendocrine cancers.

### Example 2 Study Design of Extended Intravenous Infusion of AMG 757 in Phase 1 Study Evaluating the Safety, Tolerability and Pharmacokinetics of AMG 757 in Subjects with Small Cell Lung Cancer

Study 20160232 is an open-label, ascending, multiple dose, phase 1 study evaluating AMG 757 in subjects with SCLC. There are two indications for this Study: A: Relapsed/refractory small cell lung cancer (RR SCLC) and B: Extensive disease SCLC (ED SCLC).

The Primary, Secondary, Exploratory Endpoints and the key Inclusion and Exclusion Criteria of the study are listed in Example 1.

To reduce the incidence of CRS, the cycle 1 dosing schedule is adapted to administer AMG 757 for a longer period of infusion duration as described herein. Specifically, AMG 757 is administered by extended intravenous (eIV) infusion (e.g., infusion duration range from 2 to 7 days) for the cycle 1 day 1 dose to reduce the incidence and/or severity of CRS. eIV infusion dosing involves a run-in dose or target dose administered via a 2-, 3-, 5-, or 7 day eIV infusion followed by a bolus IV infusion (e.g., 60 minute IV infusions) of the step dose on day 8 (equal to the target dose) and the target dose on day 15, or followed by a bolus IV infusion of the target dose on day 15. The dose levels for the eIV approach starting on cycle 1 day 1 include a dose of from about 1 mg to about 200 mg (e.g., 30 mg or 100 mg) infused over a 72 hour duration followed by a bolus IV infusion on day 8 and day 15 or on day 15 only. Thereafter, AMG 757 is administered Q2W (e.g., starting on day 29), twice every three weeks (e.g., starting on day 22), or Q3W (e.g., starting on day 22). A summary of eIV dose schedules (cycle 1 only) is shown in the table below.

| | Cycle 1 day 1 | Cycle 1 day 8 | Cycle 1 day 15 |
|---|---|---|---|
| Extended IV infusion | Run-in or target dose | Step dose (equal | Target dose |
| | 2-, 3-, 5-, or 7-day | to target dose) | |
| | extended IV infusion | | |
| Extended IV infusion | Run-in or target dose | N/A | Target dose |
| without day 8 dosing | 2-, 3-, 5-, or 7-day | | |
| | extended IV infusion | | |

### Example 3. Extended IV Administration to Patients with SCLC and Results

SCLC patients were enrolled in the eIV cohorts for AMG 757 monotherapy dose exploration and expansion. As of Jan. 3, 2023, thirty one (31) patients were enrolled in eIV cohorts. Starting on cycle 1 day 1, 30 mg or 100 mg AMG 757 was administered over a 3-day period (72 hrs.) via eIV infusion, this was followed by a bolus infusion of AMG 757 at a dose of 100 mg on day 8 and day 15 of cycle 1, and then 100 mg once every two weeks thereafter. Among the 31 patients, 6 received 30 mg eIV administration (Cohort 26) and 25 received 100 mg eIV administration (Cohorts 27 and 31). On cycle 1 day 1, dexamethasone 8 mg IV (or equivalent) was administered 1 hour before start of AMG 757 infusion. In addition, saline (1 L) was administered over 4-5 hours starting with commencement of the AMG 757 infusion on cycle 1 day 1. Treatment results are summarized in Table 7 below.

TABLE 7
Summary of Objective Response and Tumor Shrinkage for eIV administration

| | Cohort 26 | Cohort 27&31 |
|---|---|---|
| | (N = 6) | (N = 25) |
| Confirmed Partial Response (PR) | 2 (33.3) | 10 (40.0) |
| Stable disease (SD) | 2 (33.3) | 2 (8.0) |
| Progressive discasc (PD) | 0 (0.0) | 2 (8.0) |
| Not evaluable | 2 (33.3) | 7 (28.0) |
| PD unconfirmedb | 2 (33.3) | 7 (28.0) |
| No post-baseline scan | 0 (0.0) | 4 (16.0) |
| Unconfirmed response awaiting | 0 (0.0) | 0 (0.0) |
| confirmatory scan - n (%) | | |
| Unconfirmed CR | 0 (0.0) | 0 (0.0) |
| Unconfirmed PR | 0 (0.0) | 0 (0.0) |
| Unconfirmed response and not | 1 (16.7) | 0 (0.0) |
| confirmed at next scan - n (%) | | |
| Unconfirmed CR | 0 (0.0) | 0 (0.0) |
| Unconfirmed PR | 1 (16.7) | 0 (0.0) |
| Objective response rate (ORR) | | |
| Confirmed - n (%) | 2 (33.3) | 10 (40.0) |
| 95% CI^{C} | (4.3, 77.7) | (21.1, 61.3) |
| Confirmed and unconfirmed awaiting | 2 (33.3) | 10 (40.0) |
| confirmatory scan - n (%) | | |
| 95% CI^{C} | (4.3, 77.7) | (21.1, 61.3) |
| Disease Control Rate (DCR) - n (%) | 4 (66.7) | 12 (48.0) |
| 95% CI^{C} of DCR | (22.3, 95.7) | (27.8, 68.7) |

Any Tumor Shrinkage^{d}

| | | |
|---|---|---|
| Yes | 4 (66.7) | 13 (52.0) |
| At least 30% Tumor Shrinkage^{e} | 2 (33.3) | 10 (40.0) |
| No | 2 (33.3) | 8 (32.0) |
| Missing post-baseline sum of diameter | 0 (0.0) | 4 (16.0) |

CI = Confidence Interval; N = Number of subjects in the Interim efficacy analysis set; KM = Kaplan-Meier; N1 = number of confirmed responders; N2 = number of disease control (CR + PR + SD).
The Interim efficacy analysis set is a subset of the safety analysis set. The Interim efficacy analysis set include subjects whose data cut-off date is at least 9 weeks after the first dose date.
^{a} Assessment of disease response is determined based on modified RECIST 1.1 guidelines.
^{b}“PD Unconfirmed” classifies subjects who had PD in the post-baseline scan however without further confirmation scan.
^{C}Exact 95% confidence interval is calculated using the Clopper Pearson method.
^{d}Include subjects who has any tumor shrinkage in the target lesions at post-baseline assessment.
^{e}Include subjects who has at least 30% tumor shrinkage in the target lesions at post-baseline assessment.
^{f} Min and Max for duration of response used only event observations excluded censored observations.
Snapshot date: 3 JAN. 2023. Data cutoff date: 3 JAN. 2023.

Treatment-emergent adverse events of any grade were observed in all patients in the eIV cohorts. The adverse events lead to AMG 757 dose reduction in 1 patient (3.8%) and discontinuation of the drug in 1 patient (3.8%) in Cohorts 27 and 31. They did not lead to dose reduction or discontinuation in any patient in Cohort 26.

### Example 4 Three Week Administration Regimens of AMG 757 in Phase 1 Study Evaluating the Safety, Tolerability and Pharmacokinetics of AMG 757 in Subjects with Small Cell Lung Cancer

Study 20160232 is an open-label, ascending, multiple dose, phase 1 study evaluating AMG 757 in subjects with SCLC. There are two indications for this Study: A: Relapsed/refractory small cell lung cancer (RR SCLC) and B: Extensive disease SCLC (ED SCLC).

The Primary, Secondary, Exploratory Endpoints and the key Inclusion and Exclusion Criteria of the study are listed in Example 1.

Two 21-day dose regimens of AMG 757 are investigated in subjects with SCLC. Subjects receive AMG 757 twice every three weeks of a 21-day cycle (e.g., on day 1 and day 8 of a 21-day cycle) or once every three weeks of a 21-day cycle (e.g., day 1 of the 21-day cycle) beginning in cycle 2. Step dose, e.g., first step dose on day 1, step dose equal to target dose on day 8, and target dose on day 15, is implemented in cycle 1.

Day 1 and day 8 of 21-day cycle (D1/D8): Subjects receive AMG 7571 mg on day 1, followed by the target dose of 100 mg on day 8 and day 15. Starting with cycle 2 day 1 and onwards, subjects receive AMG 757 target dose on day 1 and day 8. The starting target dose of AMG 757 is 100 mg.

Once every 3 week dosing (Q3W): Subjects receive AMG 7571 mg on cycle 1 day 1, followed by a step dose on day 8, and the target dose on day 15. Starting with cycle 2 day 1 and beyond, subjects receive the target dose Q3W. The starting Q3W target dose is 200 mg. The Q3W dosing schedule can provide improved convenience and flexibility to patients and healthcare workers. It also has a positive impact on resource utilization (e.g., taking up fewer days of a month in a treatment center). Subjects in the 200 mg Q3W cohort receive AMG 757 100 mg on cycle 1 day 8. The dose de-escalation target doses for AMG 757 Q3W dosing include 100 mg Q3W, and 60 mg Q3W.

As of Jan. 3, 2023, 11 patients were treated in the Q3W dosing cohort (Cohort 37) and 1 patient was treated in the day 1 and day 8 of 21-day cycle cohort (Cohort 38). Briefly for the Q3W dosing, 1 mg AMG 757 was administered on cycle 1 day 1, followed by 100 mg on cycle 1 day 8, and 200 mg on cycle 1 day 15. Starting in cycle 2 day 1 and beyond, 200 mg AMG 757 was administered Q3W. For the D1/D8 dosing, 1 mg AMG 757 was administered on cycle 1 day 1, followed by 100 mg on cycle 1 day 8 and day 15. Starting in cycle 2 day 1 and beyond, 100 mg AMG 757 was administered on D1/D8 of 21-day cycle. The response of the patient who had D1/D8 dosing was stable disease. Treatment results for patients who had Q3W dosing are summarized in Table 8 below.

TABLE 8
Summary of Objective Response and Tumor
Shrinkage for AMG 757 Q3W administration

Q3W Dosing
(N = 11)

Best overall response^{a} - n (%)

| | |
|---|---|
| Confirmed Complete Response (CR) | 0 (0.0) |
| Confirmed Partial Response (PR) | 3 (27.3) |
| Stable disease (SD) | 3 (27.3) |
| Progressive disease (PD) | 0 (0.0) |
| Not evaluable | 2 (18.2) |
| PD unconfirmed^{b} | 2 (18.2) |
| No post-baseline scan | 3 (27.3) |
| Unconfirmed response awaiting confirmatory | 0 (0.0) |
| scan - n (%) | |
| Unconfirmed CR | 0 (0.0) |
| Unconfirmed PR | 0 (0.0) |
| Unconfirmed response and not confirmed at next | 1 (9.1) |
| scan - n (%) | |
| Unconfirmed CR | 0 (0.0) |
| Unconfirmed PR | 1 (9.1) |

Objective response rate (ORR)

| | |
|---|---|
| Confirmed - n (%) | 3 (27.3) |
| 95% CI^{C} | (6.0, 61.0) |
| Confirmed and unconfirmed awaiting | 3 (27.3) |
| confirmatory scan - n (%) | |
| 95% CI^{C} | (6.0, 61.0) |
| Disease Control Rate (DCR) - n (%) | 6 (54.5) |
| 95% CI^{C} of DCR | (23.4, 83.3) |

Any Tumor Shrinkage^{d}

| | |
|---|---|
| Yes | 6 (54.5) |
| At least 30% Tumor Shrinkage^{e} | 4 (36.4) |
| No | 3 (27.3) |
| Missing post-baseline sum of diameter | 2 (18.2) |

CI = Confidence Interval; N = Number of subjects in the Interim efficacy analysis set; KM = Kaplan-Meier; N1 = number of confirmed responders; N2 = number of disease control (CR + PR + SD).
The Interim efficacy analysis set is a subset of the safety analysis set. The Interim efficacy analysis set include subjects whose data cut-off date is at least 9 weeks after the first dose date.
^{a}Assessment of disease response is determined based on modified RECIST 1.1 guidelines.
^{b}“PD Unconfirmed” classifies subjects who had PD in the post-baseline scan however without further confirmation scan.
^{C}Exact 95% confidence interval is calculated using the Clopper Pearson method.
^{d}Include subjects who has any tumor shrinkage in the target lesions at post-baseline assessment.
^{e}Include subjects who has at least 30% tumor shrinkage in the target lesions at post-baseline assessment.
^{f} Min and Max for duration of response used only event observations excluded censored observations.
Snapshot date: 3 JAN. 2023. Data cutoff date: 3 JAN. 2023.

Treatment-emergent adverse events of any grade were observed in 10 patients (83.3%) in Cohort 37, dose of AMG 757 was reduced in 1 patient and the drug was discontinued in 1 patient due to the adverse events. Treatment-emergent adverse events of any grade were observed in 3 patients (100%) in Cohort 38, no dose reduction or discontinuation was made.

### Example 5 a Phase 1B Study Evaluating the Safety and Efficacy of First Line Tarlatamab (AMG 757) in Combination with Carboplatin, Etoposide, and Pd-L1 Inhibitor in Subjects with Extensive Stage Small Cell Lung Cancer

This study (Study 20200469) is a phase 1b, multicenter, open-label study evaluating the safety, tolerability, PK, pharmacodynamics (PD), and preliminary efficacy of first-line tarlatamab in combination with standard of care chemo-immunotherapy in subjects with ES-SCLC. Tarlatamab is evaluated in combination with induction chemotherapy plus anti-PD-L1 (e.g., atezolizumab) followed by maintenance cycles of tarlatamab plus anti-PD-L1 (e.g., atezolizumab), and also as maintenance only therapy with tarlatamab given in combination with anti-PD-L1 (e.g., atezolizumab) following standard of care chemo immunotherapy. The indication for this study is SCLC.

Objectives and endpoints of the study are listed in Table 9 below.

TABLE 9

| Objectives | Endpoints |
|---|---|
| Primary | |
| To evaluate the safety, tolerability, and | Dose-limiting toxicities (DLTs), |
| determine the recommended phase 2 dose | treatment-emergent and treatment-related |
| (RP2D) and/or maximum tolerated dose | adverse events, changes in vital signs, |
| (MTD) of tarlatamab in combination with | electrocardiograms (ECGs), and clinical |
| programmed death ligand 1 (PD-L1) | laboratory tests |
| inhibition with or without chemotherapy | |
| Secondary | |
| To evaluate the 6-month progression-free | PFS, defined as the time from the first |
| survival (PFS), objective response rate | dose of investigational product (IP) until |
| (ORR), duration of response (DOR), | the first documentation of radiologic |
| disease control, and overall survival (OS) | disease progression or death due to any |
| of tarlatamab in combination with PD-L1 | cause, whichever occurs first in the |
| inhibition and chemotherapy | absence of subsequent anticancer therapy. |
| | PFS will be censored at the last evaluable |
| | post-baseline tumor assessment prior to |
| | subsequent anticancer therapy; otherwise, |
| | at the first dose of IP. Progression will be |
| | based on modified Response Evaluation |
| | Criteria in Solid Tumors version 1.1 |
| | (RECIST v1.1). |
| | Objective response, defined as best |
| | overall response (BOR) of complete |
| | response (CR) or partial response (PR) |
| | based on modified RECIST v1.1. |
| | DOR, defined as the time from the first |
| | documentation of objective response until |
| | the first documentation of disease |
| | progression or death due to any cause, |
| | whichever occurs first. Only subjects who |
| | have achieved objective response will be |
| | evaluated for DOR. Progression |
| | censoring rules are per protocol |
| | Disease control, defined as objective |
| | response or stable disease per modified |
| | RECIST v1.1. |
| | OS |
| To characterize the pharmacokinetics | Serum concentrations of tarlatamab |
| (PK) of tarlatamab during induction and | |
| maintenance | |
| Exploratory | |
| To investigate exploratory biomarkers by | Quantification of biomarker expression at |
| biochemical and/or genetic analysis of | protein, RNA, and DNA level as |
| blood and/or tumor samples | appropriate |
| To characterize the immunogenicity of | Incidence of anti-tarlatamab antibody |
| tarlatamab in combination with PD-L1 | formation |
| inhibition with or without chemotherapy | |

Key Inclusion and Exclusion Criteria are summarized below.

### Key Inclusion Criteria

Age≥18 years
Subjects with histologically or cytologically confirmed ES SCLC and no prior systemic treatment for ES-SCLC other than first-line therapy described below. Subjects with prior treatment for limited stage SCLC are permitted
   Parts 1 to 4 and 7: Subject must have received 1 cycle of platinum chemotherapy, etoposide, and PD-L1 inhibitor. Subjects who did not have access to PD-L1 inhibitor are eligible.
   Parts 5, 6, 8, and 9: Subjects must have received between 4 and 6 cycles of first-line platinum chemotherapy, etoposide, and PD-L1 inhibitor and not have experienced disease progression. If there is no access to first-line PD-L1 inhibitor, subjects who received 4 to 6 cycles of platinum chemotherapy plus etoposide are eligible.
Measurable disease by modified Response Evaluation Criteria in Solid Tumors (RECIST) 1.1 (Except Parts 5, 6, 8, & 9).
Eastern Cooperative Oncology Group 0 to 1
Subjects with treated asymptomatic brain metastases are eligible provided they meet the criteria per protocol
Adequate organ function per local laboratory, defined as follows:
   Absolute neutrophil count≥1.5×10⁹/L
   Platelet count≥100×10⁹/L
   Hemoglobin≥9 g/dL
   Estimated glomerular filtration rate based on Modification of Diet in Renal Disease calculation>60 mL/min/1.73 m²
   Aspartate aminotransferase and alanine aminotransferase≤3× upper limit of normal (ULN) (or ≤5×ULN for subjects with liver involvement)
   Total bilirubin≤1.5×ULN (or <2×ULN for subjects with liver metastases)
   Prothrombin time (PT)/international normalized ratio and partial thromboplastin time or activated partial thromboplastin time≤1.5× institutional ULN
Pulmonary function:
   No clinically significant pleural effusion on study day 1. Treatment of pleural effusions to meet eligibility are permitted.
   Baseline oxygen saturation>90% on room air.
Cardiac function:
   Cardiac ejection fraction>50%

### Key Exclusion Criteria

Subjects are Excluded from the Study if any of the Following Criteria Apply:
Untreated or symptomatic brain metastases and/or leptomeningeal disease
History or evidence of interstitial lung disease or active, noninfectious pneumonitis
Has a diagnosis of immunodeficiency or is receiving systemic steroid therapy or any other form of immunosuppressive therapy within 7 days prior to the first dose of study treatment
History of any immune-related colitis
Subject with symptoms and/or clinical signs and/or radiographic signs that indicate an acute and/or uncontrolled active systemic infection within 7 days prior to the first dose of study treatment. History of hypophysitis or pituitary dysfunction
History of arterial thrombosis (eg, stroke or transient ischemic attack) within 6 months of enrollment
Myocardial infarction and/or symptomatic congestive heart failure (New York Heart Association>class II) or unstable angina within 6 months of study day 1. Unstable cardiac arrhythmia within 3 months of study day 1. Clinically significant pericardial effusion
Active autoimmune disease that has required systemic treatment (except replacement therapy) within the past 2 years or any other diseases requiring immunosuppressive therapy while on study. Subjects with Type I diabetes, vitiligo, psoriasis, hypo- or hyper-thyroid disease not requiring immunosuppressive treatment are permitted
Live vaccine therapy within 4 weeks prior to study drug administration
History of solid organ transplantation
History or evidence of any other clinically significant disorder, condition or disease that, in the opinion of the investigator or Amgen physician, if consulted, would pose a risk to subject safety or interfere with the study evaluation, procedures or completion

### Example 6 Study Design and Results of Study 2020469

Overall Design: This is a phase 1b, multicenter, open-label study evaluating the safety, tolerability, PK, pharmacodynamics (PD), and preliminary efficacy of first-line tarlatamab in combination with standard of care chemo immunotherapy in subjects with ES-SCLC. Tarlatamab is evaluated in combination with induction chemotherapy plus anti-PD-L1 (e.g., atezolizumab) followed by maintenance cycles of tarlatamab plus anti-PD-L1 (e.g., atezolizumab), and also as maintenance only therapy with tarlatamab given in combination with anti-PD-L1 (e.g., atezolizumab) following standard of care chemo-immunotherapy

Tarlatamab is administered as a short-term intravenous (IV) infusion over 60 minutes followed by a flush. To mitigate the risk of cytokine release syndrome (CRS), a step dosing approach is implemented during the first cycle of tarlatamab administration in each part. Three different dosing schedules of tarlatamab are evaluated: once every 2 weeks (Q2W), on day 1 and day 8 of a 21-day cycle (D1/D8), and once every 3 weeks (Q3W). With all dosing schedules, tarlatamab administration begin with 1 mg given on cycle 1 day 1 followed by one or more step doses to reach the target dose. Premedication with dexamethasone 8 mg IV (or equivalent dose of other corticosteroids) is administered within 1 hour prior to all first cycle doses of tarlatamab. Prophylactic IV hydration (e.g., 500 to 1000 mL saline administered over approximately 4 to 5 hours) will also be administered after all tarlatamab cycle 1 doses.

The study consists of 9 parts and includes dose escalation (Parts 1, 2, 3, and 5) and dose expansion (Parts 4, 6, 7, 8, and 9):

Part 1 (Induction tarlatamab D1/D8 and Maintenance tarlatamab Q2W): tarlatamab initiates with step dosing in cycle 1 in combination with atezolizumab, carboplatin, and etoposide followed by tarlatamab D1/D8 in cycles 2 and 3 in combination with atezolizumab, carboplatin, and etoposide. From cycle 4 onwards, subjects receive maintenance cycles of tarlatamab Q2W+ atezolizumab every 4 weeks (Q4W).

The treatment regimen for Part 1 is listed below:

Induction: Cycle 1 (21 day cycle): Atezolizumab 1200 mg IV followed by carboplatin to match area under the concentration-time curve (AUC) 5 IV, and then etoposide 100 mg/m² IV on day 1. Tarlatamab is given following the completion of chemotherapy. Tarlatamab D1/D8 is initiated with 1-step dosing with tarlatamab 1 mg IV on cycle 1 day 1. Etoposide 100 mg/m² IV is administered on day 2 and day 3. Tarlatamab D1/D8 target dose is given on cycle 1 day 8 and day 15.

Cycles 2 and 3 (21 day cycles): Atezolizumab 1200 mg IV followed by carboplatin to match AUC 5 IV and etoposide 100 mg/m² IV on day 1. Tarlatamab target dose is administered following completion of chemotherapy on day 1. Etoposide 100 mg/m² IV is also administered on days 2 and 3. Tarlatamab target dose is also administered on day 8.

Maintenance: Cycle 4+(28 day cycles): Tarlatamab Q2W target dose IV on days 1 and 15. Atezolizumab 1680 mg IV Q4W on day 1.

Part 1 includes 1 or more of the following dose levels of tarlatamab (Table 10-1) in combination with a fixed dose of atezolizumab, and carboplatin and etoposide administered per standard of care.

TABLE 10-1
Part 1 Tarlatamab dosing

| | Cycle 1^{a} | Cycle 1^{a} | Cycle 1^{a} | Cycles 2 and 3^{a} | Cycle 4+^{b} |
|---|---|---|---|---|---|
| Dose Cohort | Day 1 mg | Day 8 mg | Day 15 mg | Days 1 and | Days 1 and |
| Level | (IV) | (IV) | (IV) | 8 mg (IV) | 15 mg (IV) |
| −1.1^{c} | N/A | 1 | 10 | 10 | 10 |
| 1.1 | 1 | 10 | 10 | 10 | 10 |
| 1.2 | 1 | 30 | 30 | 30 | 30 |
| 1.3 | 1 | 100 | 100 | 100 | 100 |

IV: intravenous; N/A: not applicable
^{a}21-day cycle
^{b}28-day cycle
^{c}Dose Cohort Level -1.1 tarlatamab initiate with first step dose of 1 mg on cycle 1 day 8 followed by 10 mg target dose on cycle 1 day 15.

Part 2 (Induction tarlatamab Q3W and Maintenance tarlatamab Q3W): tarlatamab initiate with step dosing in cycle 1 in combination with atezolizumab, carboplatin, and etoposide followed by tarlatamab Q3W in cycles 2 and 3 in combination with atezolizumab, carboplatin, and etoposide. From cycle 4 onwards, subjects receive maintenance cycles of tarlatamab Q3W+ atezolizumab Q3W.

The treatment regimen for Part 2 is listed below:

Induction: Cycle 1 (21-day cycle): Atezolizumab 1200 mg IV followed by carboplatin to match AUC 5 IV and then etoposide 100 mg/m² IV on day 1. Tarlatamab is given following the completion of chemotherapy. Tarlatamab Q3W is initiated with tarlatamab first step dose (1 mg) on cycle 1 day 1. Etoposide 100 mg/m² IV is administered on cycle 1 day 2 and day 3. Tarlatamab Q3W target dose is administered on cycle 1 day 8 and on cycle 1 day 15 (see Table 10-2). Alternatively, tarlatamab Q3W target dose is administered on cycle 1 day 8 and no treatment on cycle 1 day 15 (see Table 10-2).

Cycles 2 and 3 (21-day cycles): Atezolizumab 1200 mg IV followed by carboplatin to match AUC 5 and then etoposide 100 mg/m² IV on day 1. Tarlatamab Q3W target dose is administered following completion of chemotherapy on day 1. Etoposide 100 mg/m² IV is also administered on days 2 and 3.

Maintenance: Cycle 4+(21-day cycles): Atezolizumab 1200 mg IV on day 1 followed by tarlatamab Q3W target dose on day 1 of a 21-day cycle.

TABLE 10-2
Part 2 Tarlatamab dosing

| | Cycle 1 | Cycle 1 | Cycle 1 | Cycle 2+ |
|---|---|---|---|---|
| Dose Cohort | Day 1 | Day 8 | Day 15 | Day 1 |
| Level | (mg) IV | (mg) IV | (mg) IV | (mg) IV |
| −2.1^{a} | N/A | 1 | 20 | 20 |
| 2.1 | 1 | 20 | 20 or No | 20 |
| | | | Treatment | |
| 2.2 | 1 | 60 | 60 mg or No | 60 |
| | | | Treatment | |
| 2.3^{b} | 1 | 100 | 200 mg or No | 200^{c} |
| | | | Treatment | |

IV: intravenous; N/A: not applicable; Q3W: every 3 weeks
^{a}Dose level -2.1 tarlatamab initiates with 1st step dose on cycle 1 day 8 of 1 mg, target dose on cycle 1 day 15 of 20 mg, and Q3W target dose given on cycle 2 day 1 of 20 mg.
^{b}Dose level 2.3 tarlatamab initiates with 1st step dose of 1 mg on day 1, 2nd step dose of 100 mg on day 8, and target dose of 200 mg on day 15.
^{c}Tarlatamab 100 mg Q3W may be explored as an alternative dose level if 200 mg Q3W is not deemed safe. Tarlatamab is given at 1 mg on day 1, 100 mg on day 8, and 100 mg on day 15, and 100 mg on cycle 2 day 1.

In parts 1 and 2, depending on observed safety data, the following can occur: 1) dose de-escalation to the next lowest dose cohort level, 2) additional enrollment to the current dose cohort level, or 3) dose escalation to the next highest dose cohort level or initiation of enrollment in dose expansion. Re-escalation to the next higher dose cohort level is allowed, as appropriate. If re-escalation occurs, alternative (intermediate) dose cohort levels is explored per the recommendations of the Dose Level Review Meetings (DLRM), including adjusting the dose of tarlatamab or adjusting the day of tarlatamab administration. Part 1 Dose Level 1.3 and Part 2 Dose Level 2.3 are explored if the tarlatamab monotherapy dose of 100 mg D1/D8 and 200 mg Q3W are considered safe.

Part 3 (Induction tarlatamab D1/D8 and Maintenance tarlatamab Q3W): tarlatamab initiate with step dosing in cycle 1 in combination with atezolizumab, carboplatin, and etoposide followed by tarlatamab D1/D8 in cycles 2 and 3 in combination with atezolizumab, carboplatin, and etoposide. From cycle 4 onwards, subjects receive maintenance cycles of tarlatamab Q3W+ atezolizumab Q3W.

The treatment regimen for Part 3 is listed below:

Induction: Cycle 1 (21-day cycle): Atezolizumab 1200 mg IV followed by carboplatin to match AUC 5 IV, and then etoposide 100 mg/m² IV on day 1. Tarlatamab is given following the completion of chemotherapy. Tarlatamab D1/D8 highest safe and tolerated dose is initiated with step dosing with 1 mg on cycle 1 day 1. Etoposide 100 mg/m² is administered on day 2 and day 3. Tarlatamab is administered on cycle 1 day 8, and day 15.

Cycles 2 and 3 (21-day cycles): Atezolizumab 1200 mg IV followed by carboplatin to match AUC 5 IV and then etoposide 100 mg/m² IV on day 1. Tarlatamab D1/D8 target dose is administered following completion of chemotherapy on day 1. Etoposide 100 mg/m²IV is also administered on days 2 and 3. Tarlatamab target dose is also administered on day 8.

Maintenance: Cycle 4+(21-day cycles): Atezolizumab 1200 mg IV on day 1 followed by tarlatamab Q3W target dose on day 1 of each 21 day cycle.

TABLE 10-3
Part 3 Tarlatamab dosing

| | Cycles 1 to | Cycle 4+ |
|---|---|---|
| Dose Cohort | 3 (mg) | Day 1 (mg) |
| Level | IV | IV |
| 3.1 | Selected tarlatamab | 20 |
| 3.2 | D1/D8 dose level | 60 |
| 3.3 | from Part 1 | 200 |

Part 4 (Expansion of Part 1, Part 2, or Part 3)

Part 5 (Maintenance tarlatamab Q2W plus atezolizumab): tarlatamab initiate with step dosing in cycle 1 in combination with atezolizumab. From cycle 2 onwards, subjects receive tarlatamab Q2W+ atezolizumab Q4W.

Part 5 begins with tarlatamab Dose Level 5.1 (Table 10-4). Enrollment can begin at Dose Level 5.2 or 5.3 if the lower dose level combinations have been declared safe in Part 1 or Part 2.

The treatment regimen for Part 5 is listed below:

Cycle 1 (28 day cycle): Atezolizumab 1680 mg IV on day 1 followed by tarlatamab. Tarlatamab is initiated in cycle 1 with step dosing with tarlatamab 1 mg IV on cycle 1 day 1, followed by tarlatamab Q2W target dose IV on day 8 and 15.

Cycle 2+(28 day cycles): Atezolizumab 1680 mg IV Q4W on day 1 followed by tarlatamab target dose on day 1 and day 15 from cycle 2 onwards.

TABLE 10-4
Tarlatamab dosing

| | Cycle 1 | Cycle 1 | Cycle 1 | Cycle 2+ |
|---|---|---|---|---|
| Dose Cohort | Day 1 (mg) | Day 8 (mg) | Day 15 (mg) | Days 1 and 15 (mg) |
| Level | IV | IV | IV | IV |
| 5.1 | 1 | 10 | 10 | 10 |
| 5.2 | 1 | 30 | 30 | 30 |
| 5.3 | 1 | 100 | 100 | 100 |

Part 6 (Expansion of Part5)

Part 7: Will be an expansion of the combination dose selected for expansion from Part 1, 2, or 3 using durvalumab as the PD-L1 inhibitor. Durvalumab will be administered at a dose of 1500 mg IV once every 4 weeks or 1500 mg every 3 weeks.

Part 8: Will be an expansion of the combination dose cohort selected for expansion from Part 5 using durvalumab as the PD-L1 inhibitor. Durvalumab will be given at a dose of 1500 mg IV once every 4 weeks.

Part 9: Will be an expansion cohort of maintenance tarlatamab plus durvalumab each given every 3 weeks. The tarlatamab Q3W dose will be selected from one of the dose levels in Table 10-2. Durvalumab will be given at a dose of 1500 mg every 3 weeks.

Dose escalation/de-escalation recommendations is guided by a modified toxicity probability interval-2 (mTPI-2) model (Guo et al, 2017) with a target toxicity probability of 30%, equivalence toxicity interval of (25%, 35%) and probability of overdosing of 95%. Beta (1, 1) is used as a prior distribution.

### Results

As of Jan. 3, 2023, a total of 15 patients were enrolled (5 patients in Part 2 cohort 2.1, 2 subjects in cohort −2.1, and 8 patients in cohort 5.1). Fourteen patients received tarlatamab in Parts 2 and 5 dose exploration (5 patients in Part 2 cohort 2.1, 1 patient in Part 2 cohort −2.1, and 8 patients in Part 5 cohort 5.1) of the Study. Step-dosing was utilized in both Parts. Specifically, for Part 2 cohort 2.1, 1 mg (run-in dose) was administered to patients on day 1, cycle 1 followed by 20 mg (target dose) on day 8 and day 15 in cycle 1, and then 20 mg Q3W thereafter. For Part 2 cohort −2.1, 1 mg (run-in dose) was administered to the patient on day 8, cycle 1 followed by 20 mg (target dose) on day 15 in cycle 1, and then 20 mg Q3W thereafter. One subject in cohort −2.1 received chemotherapy and atezolizumab on day 1 cycle 1 but did not receive tarlatamab due to an unrelated infection and ended study. For Part 5, 1 mg (run-in dose) was administered to patients on day 1 in cycle 1 followed by 10 mg (target dose) on day 8 and day 15 in cycle, and then 10 mg Q2W thereafter. Atezolizumab and chemotherapy were administered as described above.

For Part 2, of the 5 subjects that received tarlatamab in cohort 2.1, 5 subjects had an imaging assessment for response. Two patients had a confirmed partial response (PR), 1 subject had an unconfirmed PR, and 2 patients had stable disease (SD). The one subject that received tarlatamab in cohort −2.1 had an unconfirmed PR. The total responses for Part 2 were 2/6 confirmed PR (33%), 2/6 unconfirmed PR (33%), and 2 SD (33%).

For Part 5, of the 8 subjects that received tarlatamab, 5 subjects had an imaging assessment for response. Of these 5 subjects, 3 subjects had SD (60%), and 2 subjects had disease progression (40%). 3 subjects had not yet had an imaging assessment.

Due to the known high response rate to initial standard of care treatment, the primary response assessment for the combination of tarlatamab with chemotherapy and PD-L1 inhibitor will be 6-month progression free survival and overall survival and it is too early to evaluate these measures. Overall, the responses thus far are encouraging for the tarlatamab combination regimen.

Of the 15 enrolled patients, treatment-emergent adverse events were observed in 14 (93.3%) patients. Treatment related-emergent adverse events>grade 2 were observed in 13 (86.7%) patients. In Part 2 cohort 2.1, 2 subjects discontinued treatment due to treatment related adverse events. 1 subject experienced a grade 3 ICANS event of seizure and 1 subject experienced grade 4 thrombocytopenia related to chemotherapy. Cytokine Release Syndrome occurred in 7 of 15 subjects (46.7%). Six subjects experienced grade 1 CRS and one subject experienced grade 2 CRS.

Table 11 below lists the sequences referenced in this application.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

| | | Form | |
|---|---|---|---|
| SEQ | Desig- | at/ | |
| ID NO | nation | source | Sequence |
| 1 | DLL3-4 | VH | SYYWS |
| | | CDR1 | |
| 2 | DLL3-4 | VH | YVYYSGTTNYNPSLKS |
| | | CDR2 | |
| 3 | DLL3-4 | VH | IAVTGFYFDY |
| | | CDR3 | |
| 4 | DLL3-4 | VL | RASQRVNNNYLA |
| | | CDR1 | |
| 5 | DLL3-4 | VL | GASSRAT |
| | | CDR2 | |
| 6 | DLL3-4 | VL | QQYDRSPLT |
| | | CDR3 | |
| 7 | DLL3-4 | VH | QVQLQESGPGLVKPSETLSLTCTVSGGSISSYYWSWIRQPPGKGLEWIGY |
| | | | VYYSGTTNYNPSLKSRVTISVDTSKNQFSLKLSSVTAADTAVYYCASIAV |
| | | | TGFYFDYWGQGTLVTVSS |
| 8 | DLL3-4 | VL | EIVLTQSPGTLSLSPGERVTLSCRASQRVNNNYLAWYQQRPGQAPRLLIY |
| | | | GASSRATGIPDRFSGSGSGTDFTLTISRLEPEDFAVYYCQQYDRSPLTFGG |
| | | | GTKLEIK |
| 9 | DLL3-4 | scFv | QVQLQESGPGLVKPSETLSLTCTVSGGSISSYYWSWIRQPPGKGLEWIGY |
| | | | VYYSGTTNYNPSLKSRVTISVDTSKNQFSLKLSSVTAADTAVYYCASIAV |
| | | | TGFYFDYWGQGTLVTVSSGGGGSGGGGSGGGGSEIVLTQSPGTLSLSPG |
| | | | ERVTLSCRASQRVNNNYLAWYQQRPGQAPRLLIYGASSRATGIPDRFSG |
| | | | SGSGTDFTLTISRLEPEDFAVYYCQQYDRSPLTFGGGTKLEIK |
| 10 | DLL3- | bispecific | QVQLQESGPGLVKPSETLSLTCTVSGGSISSYYWSWIRQPPGKGLEWIGY |
| | 4xI2C | | VYYSGTTNYNPSLKSRVTISVDTSKNQFSLKLSSVTAADTAVYYCASIAV |
| | | | TGFYFDYWGQGTLVTVSSGGGGSGGGGSGGGGSEIVLTQSPGTLSLSPG |
| | | | ERVTLSCRASQRVNNNYLAWYQQRPGQAPRLLIYGASSRATGIPDRFSG |
| | | | SGSGTDFTLTISRLEPEDFAVYYCQQYDRSPLTFGGGTKLEIKSGGGGSE |
| | | | VQLVESGGGLVQPGGSLKLSCAASGFTFNKYAMNWVRQAPGKGLEWV |
| | | | ARIRSKYNNYATYYADSVKDRFTISRDDSKNTAYLQMNNLKTEDTAVY |
| | | | YCVRHGNFGNSYISYWAYWGQGTLVTVSSGGGGSGGGGSGGGGSQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVF |
| | | | GGGTKLTVL |
| 11 | DLL3- | VH | QVQLQESGPGLVKPSETLSLTCTVSGGSISSYYWSWIRQPPGKCLEWIGY |
| | 4- | | VYYSGTTNYNPSLKSRVTISVDTSKNQFSLKLSSVTAADTAVYYCASIAV |
| | 001 | | TGFYFDYWGQGTLVTVSS |
| | (G44C) | | |
| 12 | DLL3- | VL | EIVLTQSPGTLSLSPGERVTLSCRASQRVNNNYLAWYQQRPGQAPRLLIY |
| | 4- | | GASSRATGIPDRFSGSGSGTDFTLTISRLEPEDFAVYYCQQYDRSPLTFGC |
| | 001 | | GTKLEIK |
| | (G234C) | | |
| 13 | DLL3- | scFv | QVQLQESGPGLVKPSETLSLTCTVSGGSISSYYWSWIRQPPGKCLEWIGY |
| | 4- | | VYYSGTTNYNPSLKSRVTISVDTSKNQFSLKLSSVTAADTAVYYCASIAV |
| | 001 | | TGFYFDYWGQGTLVTVSSGGGGSGGGGSGGGGSEIVLTQSPGTLSLSPG |
| | (G44C- | | ERVTLSCRASQRVNNNYLAWYQQRPGQAPRLLIYGASSRATGIPDRFSG |
| | G243C) | | SGSGTDFTLTISRLEPEDFAVYYCQQYDRSPLTFGCGTKLEIK |
| 14 | DLL3- | bispecific | QVQLQESGPGLVKPSETLSLTCTVSGGSISSYYWSWIRQPPGKCLEWIGY |
| | 4- | | VYYSGTTNYNPSLKSRVTISVDTSKNQFSLKLSSVTAADTAVYYCASIAV |
| | 001 | | TGFYFDYWGQGTLVTVSSGGGGSGGGGSGGGGSEIVLTQSPGTLSLSPG |
| | (CC) | | ERVTLSCRASQRVNNNYLAWYQQRPGQAPRLLIYGASSRATGIPDRFSG |
| | xI2C | | SGSGTDFTLTISRLEPEDFAVYYCQQYDRSPLTFGCGTKLEIKSGGGGSEV |
| | | | QLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYY |
| | | | CVRHGNFGNSYISYWAYWGQGTLVTVSSGGGGSGGGGSGGGGSQTVV |
| | | | TQEPSLTVSPGGTVTLTCGSSTGAVTSGNYPNWVQQKPGQAPRGLIGGT |
| | | | KFLAPGTPARFSGSLLGGKAALTLSGVQPEDEAEYYCVLWYSNRWVFG |
| | | | GGTKLTVL |
| 15 | CDR-L1 | artificial | GSSTGAVTSGNYPN |
| | of | | |
| | I2C | | |
| 16 | CDR-L2 | artificial | GTKFLAP |
| | of | | |
| | I2C | | |
| 17 | CDR-L3 | artificial | VLWYSNRWV |
| | of | | |
| | I2C | | |
| 18 | CDR-H1 | artificial | KYAMN |
| | of | | |
| | I2C | | |
| 19 | CDR-H2 | artificial | RIRSKYNNYATYYADSVKD |
| | of | | |
| | I2C | | |
| 20 | CDR-H3 | artificial | HGNFGNSYISYWAY |
| | of | | |
| | I2C | | |
| 21 | VH | artificial | EVQLVESGGGLVQPGGSLKLSCAASGFTFNKYAMNWVRQAPGKGLEW |
| | of | | VARIRSKYNNYATYYADSVKDRFTISRDDSKNTAYLQMNNLKTEDTAV |
| | I2C | | YYCVRHGNFGNSYISYWAYWGQGTLVTVSS |
| 22 | VL | artificial | QTVVTQEPSLTVSPGGTVTLTCGSSTGAVTSGNYPNWVQQKPGQAPRGL |
| | of | | IGGTKFLAPGTPARFSGSLLGGKAALTLSGVQPEDEAEYYCVLWYSNRW |
| | I2C | | VFGGGTKLTVL |
| 23 | VH- | artificial | EVQLVESGGGLVQPGGSLKLSCAASGFTFNKYAMNWVRQAPGKGLEW |
| | VL | | VARIRSKYNNYATYYADSVKDRFTISRDDSKNTAYLQMNNLKTEDTAV |
| | of | | YYCVRHGNFGNSYISYWAYWGQGTLVTVSSGGGGSGGGGSGGGGSQT |
| | I2C | | VVTQEPSLTVSPGGTVTLTCGSSTGAVTSGNYPNWVQQKPGQAPRGLIG |
| | | | GTKFLAPGTPARFSGSLLGGKAALTLSGVQPEDEAEYYCVLWYSNRWV |
| | | | FGGGTKLTVL |
| 24 | DLL3- | bispecific | QVQLQESGPGLVKPSETLSLTCTVSGGSISSYYWSWIRQPPGKGLEWIGY |
| | 4 | HLE | VYYSGTTNYNPSLKSRVTISVDTSKNQFSLKLSSVTAADTAVYYCASIAV |
| | xI2C- | molecule | TGFYFDYWGQGTLVTVSSGGGGSGGGGSGGGGSEIVLTQSPGTLSLSPG |
| | scFc | | ERVTLSCRASQRVNNNYLAWYQQRPGQAPRLLIYGASSRATGIPDRFSG |
| | | | SGSGTDFTLTISRLEPEDFAVYYCQQYDRSPLTFGGGTKLEIKSGGGGSE |
| | | | VQLVESGGGLVQPGGSLKLSCAASGFTFNKYAMNWVRQAPGKGLEWV |
| | | | ARIRSKYNNYATYYADSVKDRFTISRDDSKNTAYLQMNNLKTEDTAVY |
| | | | YCVRHGNFGNSYISYWAYWGQGTLVTVSSGGGGSGGGGSGGGGSQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVF |
| | | | GGGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGKGGG |
| | | | GSGGGGSGGGGSGGGGSGGGGSGGGGSDKTHTCPPCPAPELLGGPSVFL |
| | | | FPPKPKDTLMISRTPEVTCVVVDVSHEDPEVKFNWYVDGVEVHNAKTK |
| | | | PCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHY |
| | | | TQKSLSLSPGK |
| 25 | DLL3- | bispecific | QVQLQESGPGLVKPSETLSLTCTVSGGSISSYYWSWIRQPPGKGLEWIGY |
| | 4 | HLE | VYYSGTTNYNPSLKSRVTISVDTSKNQFSLKLSSVTAADTAVYYCASIAV |
| | xI2C- | molecule | TGFYFDYWGQGTLVTVSSGGGGSGGGGSGGGGSEIVLTQSPGTLSLSPG |
| | scFc | | ERVTLSCRASQRVNNNYLAWYQQRPGQAPRLLIYGASSRATGIPDRFSG |
| | delGK | | SGSGTDFTLTISRLEPEDFAVYYCQQYDRSPLTFGGGTKLEIKSGGGGSE |
| | | | VQLVESGGGLVQPGGSLKLSCAASGFTFNKYAMNWVRQAPGKGLEWV |
| | | | ARIRSKYNNYATYYADSVKDRFTISRDDSKNTAYLQMNNLKTEDTAVY |
| | | | YCVRHGNFGNSYISYWAYWGQGTLVTVSSGGGGSGGGGSGGGGSQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVF |
| | | | GGGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGGGGS |
| | | | GGGGSGGGGSGGGGSGGGGSGGGGSDKTHTCPPCPAPELLGGPSVFLFP |
| | | | PKPKDTLMISRTPEVTCVVVDVSHEDPEVKFNWYVDGVEVHNAKTKPC |
| | | | EEQYGSTYRCVSVLTVLHQDWLNGKEYKCKVSNKALPAPIEKTISKAKG |
| | | | QPREPQVYTLPPSREEMTKNQVSLTCLVKGFYPSDIAVEWESNGQPENN |
| | | | YKTTPPVLDSDGSFFLYSKLTVDKSRWQQGNVFSCSVMHEALHNHYTQ |
| | | | KSLSLSPGK |
| 26 | DLL3- | bispecific | QVQLQESGPGLVKPSETLSLTCTVSGGSISSYYWSWIRQPPGKCLEWIGY |
| | 4- | HLE | VYYSGTTNYNPSLKSRVTISVDTSKNQFSLKLSSVTAADTAVYYCASIAV |
| | 001 | molecule | TGFYFDYWGQGTLVTVSSGGGGSGGGGSGGGGSEIVLTQSPGTLSLSPG |
| | (CC) | | ERVTLSCRASQRVNNNYLAWYQQRPGQAPRLLIYGASSRATGIPDRFSG |
| | xI2C- | | SGSGTDFTLTISRLEPEDFAVYYCQQYDRSPLTFGCGTKLEIKSGGGGSEV |
| | scFc | | QLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYY |
| | | | CVRHGNFGNSYISYWAYWGQGTLVTVSSGGGGSGGGGSGGGGSQTVV |
| | | | TQEPSLTVSPGGTVTLTCGSSTGAVTSGNYPNWVQQKPGQAPRGLIGGT |
| | | | KFLAPGTPARFSGSLLGGKAALTLSGVQPEDEAEYYCVLWYSNRWVFG |
| | | | GGTKLTVLGGGGDKTHTCPPCPAPELLGGPSVFLFPPKPKDTLMISRTPE |
| | | | VTCVVVDVSHEDPEVKFNWYVDGVEVHNAKTKPCEEQYGSTYRCVSV |
| | | | LTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGKGGGG |
| | | | SGGGGSGGGGSGGGGSGGGGSGGG27GSDKTHTCPPCPAPELLGGPSVF |
| | | | LFPPKPKDTLMISRTPEVTCVVVDVSHEDPEVKFNW28YVDGVEVHNAK |
| | | | TKPCEEQYGSTYRCVSVLTVLHQDWLNGKEYKCKVSNKALPAPIEKTIS |
| | | | KAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHN |
| | | | HYTQKSLSLSPGK |
| 27 | DLL3- | bispecific | QVQLQESGPGLVKPSETLSLTCTVSGGSISSYYWSWIRQPPGKCLEWIGY |
| | 4- | HLE | VYYSGTTNYNPSLKSRVTISVDTSKNQFSLKLSSVTAADTAVYYCASIAV |
| | 001 | molecule | TGFYFDYWGQGTLVTVSSGGGGSGGGGSGGGGSEIVLTQSPGTLSLSPG |
| | (CC) | | ERVTLSCRASQRVNNNYLAWYQQRPGQAPRLLIYGASSRATGIPDRFSG |
| | xI2C- | | SGSGTDFTLTISRLEPEDFAVYYCQQYDRSPLTFGCGTKLEIKSGGGGSEV |
| | scFc | | QLVESGGGLVQPGGSLKLSCAASGFTFNKYAMNWVRQAPGKGLEWVA |
| | delGK | | RIRSKYNNYATYYADSVKDRFTISRDDSKNTAYLQMNNLKTEDTAVYY |
| | | | CVRHGNFGNSYISYWAYWGQGTLVTVSSGGGGSGGGGSGGGGSQTVV |
| | | | TQEPSLTVSPGGTVTLTCGSSTGAVTSGNYPNWVQQKPGQAPRGLIGGT |
| | | | KFLAPGTPARFSGSLLGGKAALTLSGVQPEDEAEYYCVLWYSNRWVFG |
| | | | GGTKLTVLGGGGDKTHTCPPCPAPELLGGPSVFLFPPKPKDTLMISRTPE |
| | | | VTCVVVDVSHEDPEVKFNWYVDGVEVHNAKTKPCEEQYGSTYRCVSV |
| | | | LTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGGGGSG |
| | | | GGGSGGGGSGGGGSGGGGSGGGGSDKTHTCPPCPAPELLGGPSVFLFPP |
| | | | KPKDTLMISRTPEVTCVVVDVSHEDPEVKFNWYVDGVEVHNAKTKPCE |
| | | | EQYGSTYRCVSVLTVLHQDWLNGKEYKCKVSNKALPAPIEKTISKAKGQ |
| | | | PREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQK |
| | | | SLSLSPGK |
| 28 | Human | human | MVSPRMSGLLSQTVILALIFLPQTRPAGVFELQIHSFGPGPGPGAPRSPCS |
| | DLL3 | | ARLPCRLFFRVCLKPGLSEEAAESPCALGAALSARGPVYTEQPGAPAPDL |
| | | | PLPDGLLQVPFRDAWPGTFSFIIETWREELGDQIGGPAWSLLARVAGRRR |
| | | | LAAGGPWARDIQRAGAWELRFSYRARCEPPAVGTACTRLCRPRSAPSRC |
| | | | GPGLRPCAPLEDECEAPLVCRAGCSPEHGFCEQPGECRCLEGWTGPLCT |
| | | | VPVSTSSCLSPRGPSSATTGCLVPGPGPCDGNPCANGGSCSETPRSFECTC |
| | | | PRGFYGLRCEVSGVTCADGPCFNGGLCVGGADPDSAYICHCPPGFQGSN |
| | | | CEKRVDRCSLQPCRNGGLCLDLGHALRCRCRAGFAGPRCEHDLDDCAG |
| | | | RACANGGTCVEGGGAHRCSCALGFGGRDCRERADPCAARPCAHGGRC |
| | | | YAHFSGLVCACAPGYMGARCEFPVHPDGASALPAAPPGLRPGDPQRYLL |
| | | | PPALGLLVAAGVAGAALLLVHVRRRGHSQDAGSRLLAGTPEPSVHALP |
| | | | DALNNLRTQEGSGDGPSSSVDWNRPEDVDPQGIYVISAPSIYAREVATPL |
| | | | FPPLHTGRAGQRQHLLFPYPSSILSVK |
| 29 | Hu | artificial | SGVTCADGPCFNGGLCVGGADPDSAYICHCPPGFQGSNCE |
| | DLL3 | | |
| | EGF-3 | | |
| 30 | Hu | articiaL | RVDRCSLQPCRNGGLCLDLGHALRCRCRAGFAGPRCE |
| | DLL3 | | |
| | EGF-4 | | |
| 31 | Hu | human | SGVTCADGPCFNGGLCVGGADPDSAYICHCPPGFQGSNCEKRVDRCSLQ |
| | DLL3 | | PCRNGGLCLDLGHALRCRCRAGFAGPRCE |
| | EGF- | | |
| | 3 + 4 | | |
| 32 | DLL3- | bispecific | QVQLQESGPGLVKPSETLSLTCTVSGGSISSYYWSWIRQPPGKCLEWIGY |
| | 4- | HLE | VYYSGTTNYNPSLKSRVTISVDTSKNQFSLKLSSVTAADTAVYYCASIAV |
| | 001 | molecule | TGFYFDYWGQGTLVTVSSGGGGSGGGGSGGGGSEIVLTQSPGTLSLSPG |
| | (CC) | | ERVTLSCRASQRVNNNYLAWYQQRPGQAPRLLIYGASSRATGIPDRFSG |
| | xI2C- | | SGSGTDFTLTISRLEPEDFAVYYCQQYDRSPLTFGCGTKLEIKSGGGGSEV |
| | scFc | | QLVESGGGLVQPGGSLKLSCAASGFTFNKYAMNWVRQAPGKGLEWVA |
| | delGK | | RIRSKYNNYATYYADSVKDRFTISRDDSKNTAYLQMNNLKTEDTAVYY |
| | | | CVRHGNFGNSYISYWAYWGQGTLVTVSSGGGGSGGGGSGGGGSQTVV |
| | | | TQEPSLTVSPGGTVTLTCGSSTGAVTSGNYPNWVQQKPGQAPRGLIGGT |
| | | | KFLAPGTPARFSGSLLGGKAALTLSGVQPEDEAEYYCVLWYSNRWVFG |
| | | | GGTKLTVLGGGGDKTHTCPPCPAPELLGGPSVFLFPPKPKDTLMISRTPE |
| | | | VTCVVVDVSHEDPEVKFNWYVDGVEVHNAKTKPCEEQYGSTYRCVSV |
| | | | LTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGGGGSG |
| | | | GGGSGGGGSGGGGSGGGGSGGGGSDKTHTCPPCPAPELLGGPSVFLFPP |
| | | | KPKDTLMISRTPEVTCVVVDVSHEDPEVKFNWYVDGVEVHNAKTKPCE |
| | | | EQYGSTYRCVSVLTVLHQDWLNGKEYKCKVSNKALPAPIEKTISKAKGQ |
| | | | PREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQK |
| | | | SLSLSPG |
| 33 | | artificial | FPVHPDGASALPAAPPGLRPGDPQRYL |
| 34 | Bispecific | artificial | Glu Val Gln Leu Val Glu Ser Gly Gly Gly Leu Val Gln Pro Gly Gly |
| | molecule | | 1 5 10 15 |
| | | | Ser Leu Lys Leu Ser Cys Ala Ala Ser Gly Phe Thr Phe Asn Lys Tyr |
| | | | 20 25 30 |
| | | | Ala Ile Asn Trp Val Arg Gln Ala Pro Gly Lys Gly Leu Glu Trp Val |
| | | | 35 40 45 |
| | | | Ala Arg Ile Arg Ser Lys Tyr Asn Asn Tyr Ala Thr Tyr Tyr Ala Asp |
| | | | 50 55 60 |
| | | | Gln Val Lys Asp Arg Phe Thr Ile Ser Arg Asp Asp Ser Lys Asn Thr |
| | | | 65 70 75 80 |
| | | | Ala Tyr Leu Gln Met Asn Asn Leu Lys Thr Glu Asp Thr Ala Val Tyr |
| | | | 85 90 95 |
| | | | Tyr Cys Val Arg His Ala Asn Phe Gly Asn Ser Tyr Ile Ser Tyr Trp |
| | | | 100 105 110 |
| | | | Ala Tyr Trp Gly Gln Gly Thr Leu Val Thr Val Ser Ser Gly Gly Gly |
| | | | 115 120 125 |
| | | | Gly Ser Gly Gly Gly Gly Ser Gly Gly Gly Gly Ser Gln Thr Val Val |
| | | | 130 135 140 |
| | | | Thr Gln Glu Pro Ser Leu Thr Val Ser Pro Gly Gly Thr Val Thr Leu |
| | | | 145 150 155 160 |
| | | | Thr Cys Ala Ser Ser Thr Gly Ala Val Thr Ser Gly Asn Tyr Pro Asn |
| | | | 165 170 175 |
| | | | Trp Val Gln Gln Lys Pro Gly Gln Ala Pro Arg Gly Leu Ile Gly Gly |
| | | | 180 185 190 |
| | | | Thr Lys Phe Leu Val Pro Gly Thr Pro Ala Arg Phe Ser Gly Ser Lcu |
| | | | 195 200 205 |
| | | | Leu Gly Gly Lys Ala Ala Leu Thr Leu Ser Gly Val Gln Pro Glu Asp |
| | | | 210 215 220 |
| | | | Glu Ala Glu Tyr Tyr Cys Thr Leu Trp Tyr Ser Asn Arg Trp Val Phe |
| | | | 225 230 235 240 |
| | | | Gly Gly Gly Thr Lys Leu Thr Val Leu Gly Gly Gly Gly Ser Gly Gly |
| | | | 245 250 255 |
| | | | Gly Ser Glu Val Gln Leu Val Glu Ser Gly Gly Gly Leu Val Gln Pro |
| | | | 260 265 270 |
| | | | Gly Asn Ser Leu Arg Leu Ser Cys Ala Ala Ser Gly Phe Thr Phe Ser |
| | | | 275 280 285 |
| | | | Lys Phe Gly Met Ser Trp Val Arg Gln Ala Pro Gly Lys Gly Leu Glu |
| | | | 290 295 300 |
| | | | Trp Val Ser Ser Ile Ser Gly Ser Gly Arg Asp Thr Leu Tyr Ala Asp |
| | | | 305 310 315 320 |
| | | | Ser Val Lys Gly Arg Phe Thr Ile Ser Arg Asp Asn Ala Lys Thr Thr |
| | | | 325 330 335 |
| | | | Leu Tyr Leu Gln Met Asn Ser Leu Arg Pro Glu Asp Thr Ala Val Tyr |
| | | | 340 345 350 |
| | | | Tyr Cys Thr Ile Gly Gly Ser Leu Ser Val Ser Ser Gln Gly Thr Leu |
| | | | 355 360 365 |
| | | | Val Thr Val Ser Ser Gly Gly Gly Gly Ser Gly Gly Gly Ser Glu Val |
| | | | 370 375 380 |
| | | | Gln Leu Val Glu Ser Gly Gly Gly Leu Val Gln Pro Gly Gly Ser Leu |
| | | | 385 390 395 400 |
| | | | Thr Leu Ser Cys Ala Ala Ser Ser Ser Ser Val Ser Leu Leu Ser Leu |
| | | | 405 410 415 |
| | | | Ala Trp Tyr Arg Gln Ala Pro Gly Lys Lys Arg Glu Leu Val Ala Gly |
| | | | 420 425 430 |
| | | | Ile Ser Asp Asp Gly Ser Ile Val Tyr Met Asp Ser Val Lys Gly Arg |
| | | | 435 440 445 |
| | | | Phe Thr Ile Ser Arg Asp Asn Ala Lys Asn Ser Val Tyr Leu Gln Met |
| | | | 450 455 460 |
| | | | Asn Ser Leu Arg Ala Glu Asp Thr Ala Val Tyr Tyr Cys Tyr Ala Tyr |
| | | | 465 470 475 480 |
| | | | Ser Trp Ile Thr Arg Ser Pro Tyr Trp Gly Gln Gly Thr Leu Val Thr |
| | | | 485 490 495 |
| | | | Val Ser Ser His His His His His His |
| | | | 500 505 |
| 35 | Bispecific | artificial | Glu Val Gln Leu Val Glu Ser Gly Gly Gly Leu Val Gln Pro Gly Gly |
| | molecule | | 1 5 10 15 |
| | | | Ser Leu Thr Leu Ser Cys Ala Ala Ser Ser Ser Ser Val Ser Leu Leu |
| | | | 20 25 30 |
| | | | Ser Leu Ala Trp Tyr Arg Gln Ala Pro Gly Lys Lys Arg Glu Leu Val |
| | | | 35 40 45 |
| | | | Ala Gly Ile Ser Asp Asp Gly Ser Ile Val Tyr Met Asp Ser Val Lys |
| | | | 50 55 60 |
| | | | Gly Arg Phe Thr Ile Ser Arg Asp Asn Ala Lys Asn Ser Val Tyr Leu |
| | | | 65 70 75 80 |
| | | | Gln Met Asn Ser Leu Arg Ala Glu Asp Thr Ala Val Tyr Tyr Cys Tyr |
| | | | 85 90 95 |
| | | | Ala Tyr Ser Trp Ile Thr Arg Ser Pro Tyr Trp Gly Gln Gly Thr Leu |
| | | | 100 105 110 |
| | | | Val Thr Val Ser Ser Gly Gly Gly Gly Ser Gly Gly Gly Ser Glu Val |
| | | | 115 120 125 |
| | | | Gln Leu Val Glu Ser Gly Gly Gly Leu Val Gln Pro Gly Asn Ser Leu |
| | | | 130 135 140 |
| | | | Arg Leu Ser Cys Ala Ala Ser Gly Phe Thr Phe Ser Lys Phe Gly Met |
| | | | 145 150 155 160 |
| | | | Ser Trp Val Arg Gln Ala Pro Gly Lys Gly Leu Glu Trp Val Ser Ser |
| | | | 165 170 175 |
| | | | Ile Ser Gly Ser Gly Arg Asp Thr Leu Tyr Ala Asp Ser Val Lys Gly |
| | | | 180 185 190 |
| | | | Arg Phe Thr Ile Ser Arg Asp Asn Ala Lys Thr Thr Leu Tyr Leu Gln |
| | | | 195 200 205 |
| | | | Met Asn Ser Leu Arg Pro Glu Asp Thr Ala Val Tyr Tyr Cys Thr Ile |
| | | | 210 215 220 |
| | | | Gly Gly Ser Leu Ser Val Ser Ser Gln Gly Thr Leu Val Thr Val Ser |
| | | | 225 230 235 240 |
| | | | Ser Gly Gly Gly Gly Ser Gly Gly Gly Ser Glu Val Gln Leu Val Glu |
| | | | 245 250 255 |
| | | | Ser Gly Gly Gly Leu Val Gln Pro Gly Gly Ser Leu Lys Leu Ser Cys |
| | | | 260 265 270 |
| | | | Ala Ala Ser Gly Phe Thr Phe Asn Lys Tyr Ala Ile Asn Trp Val Arg |
| | | | 275 280 285 |
| | | | Gln Ala Pro Gly Lys Gly Leu Glu Trp Val Ala Arg Ilc Arg Ser Lys |
| | | | 290 295 300 |
| | | | Tyr Asn Asn Tyr Ala Thr Tyr Tyr Ala Asp Gln Val Lys Asp Arg Phe |
| | | | 305 310 315 320 |
| | | | Thr Ile Ser Arg Asp Asp Ser Lys Asn Thr Ala Tyr Leu Gln Met Asn |
| | | | 325 330 335 |
| | | | Asn Leu Lys Thr Glu Asp Thr Ala Val Tyr Tyr Cys Val Arg His Ala |
| | | | 340 345 350 |
| | | | Asn Phe Gly Asn Ser Tyr Ile Ser Tyr Trp Ala Tyr Trp Gly Gln Gly |
| | | | 355 360 365 |
| | | | Thr Leu Val Thr Val Ser Ser Gly Gly Gly Gly Ser Gly Gly Gly Gly |
| | | | 370 375 380 |
| | | | Ser Gly Gly Gly Gly Ser Gln Thr Val Val Thr Gln Glu Pro Ser Leu |
| | | | 385 390 395 400 |
| | | | Thr Val Ser Pro Gly Gly Thr Val Thr Leu Thr Cys Ala Ser Ser Thr |
| | | | 405 410 415 |
| | | | Gly Ala Val Thr Ser Gly Asn Tyr Pro Asn Trp Val Gln Gln Lys Pro |
| | | | 420 425 430 |
| | | | Gly Gln Ala Pro Arg Gly Leu Ile Gly Gly Thr Lys Phe Leu Val Pro |
| | | | 435 440 445 |
| | | | Gly Thr Pro Ala Arg Phe Ser Gly Ser Leu Leu Gly Gly Lys Ala Ala |
| | | | 450 455 460 |
| | | | Leu Thr Leu Ser Gly Val Gln Pro Glu Asp Glu Ala Glu Tyr Tyr Cys |
| | | | 465 470 475 480 |
| | | | Thr Leu Trp Tyr Ser Asn Arg Trp Val Phe Gly Gly Gly Thr Lys Leu |
| | | | 485 490 495 |
| | | | Thr Val Leu His His His His His His |
| | | | 500 505 |
| 36 | | Artificial | DIQMTQSPSAMSASVGDRVTITCRASQGISNYLVWFQQKPGKAPKRLIYAVSSL |
| | | | YSGVPSRFSGSGSGTEFTLTISSLQPEDFATYYCLQHDSYPYTFGQGTKLEIKR |
| | | | TVAAPSVFIFPPSDEQLKSGTASVVCLLNNFYPREAKVQWKVDNALQSGNSQES |
| | | | VTEQDSKDSTYSLSSTLTLSKADYEKHKVYACEVTHQGLSSPVTKSFNRGECGG |
| | | | GGSEGKSSGSGSESKSTEGKSSGSGSESKSTGGGGSQVQLVQSGAEVKKPGASV |
| | | | KVSCKASGYTFTSYYVHWVRQAPGQGLEWMVIINPGGGTTSYAQKFLGRVTMTR |
| | | | DTSTNTVYMELKSLRSEDTAVYYCARGEAVTGNYFYYGMDVWGQGTTVTVSSAS |
| | | | TKGPSVFPLAPSSKSTSGGTAALGCLVKDYFPEPVTVSWNSGALTSGVHTFPAV |
| | | | LQSSGLYSLSSVVTVPSSSLGTQTYICNVNHKPSNTKVDKRVEPKSCDKTHTCP |
| | | | PCPAPEAAGGPSVFLFPPKPKDTLMISRTPEVTCVVVDVSHEDPEVKFNWYVDG |
| | | | VEVHNAKTKPREEQYNSTYRVVSVLTVLHQDWLNGKEYKCKVSNKALPAPIEKT |
| | | | ISKAKGQPREPQVYTLPPSREEMTKNQVSLWCLVKGFYPSDIAVEWESNGQPEN |
| | | | NYKTTPPVLDSDGSFFLYSKLTVDKSRWQQGNVFSCSVMHEALHNHYTQKSLSL |
| | | | SPG |
| 37 | | Artificial | EAVVTQEPSLTVSPGGTVTLTCRSSTGAVTTSNYANWVQEKPGQLPRGLIGGTN |
| | | | KRAPWVPARFSGSLLGGKAALTLSGAQPEDEAEYFCALWYSNLWVFGGGTKLTV |
| | | | LGQPKAAPSVTLFPPSSEELQANKATLVCLISDFYPGAVKVAWKADGSPVNTGV |
| | | | ETTTPSKQSNNKYAASSYLSLTPEQWKSHRSYSCQVTHEGSTVEKTVAPAECSG |
| | | | GGGSEGKSSGSGSESKSTEGKSSGSGSESKSTGGGGSEVQLVESGGGLVQPGGS |
| | | | LKLSCAASGFTFNTYAMNWVRQAPGKGLEWVARIRSKYNNYATYYADSVKDRFT |
| | | | ISRDDSKNTAYLQMNNLKTEDTAVYYCVRHGNFGNSYVSWFAYWGQGTLVTVSA |
| | | | ASTKGPSVFPLAPSSKSTSGGTAALGCLVKDYFPEPVTVSWNSGALTSGVHTEP |
| | | | AVLQSSGLYSLSSVVTVPSSSLGTQTYICNVNHKPSNTKVDKRVEPKSCDKTHT |
| | | | CPPCPAPEAAGGPSVFLFPPKPKDTLMISRTPEVTCVVVDVSHEDPEVKFNWYV |
| | | | DGVEVHNAKTKPREEQYNSTYRVVSVLTVLHQDWLNGKEYKCKVSNKALPAPIE |
| | | | KTISKAKGQPREPQVYTLPPSREEMTKNQVSLSCAVKGFYPSDIAVEWESNGQP |
| | | | ENNYKTTPPVLDSDGSFFLVSKLTVDKSRWQQGNVFSCSVMHEALHNRFTQKSL |
| | | | SLSPG |

What is claimed:

1. A method of treating DLL3-positive cancer, comprising administering to a subject in need thereof an anti-DLL3 agent comprising the amino acid sequence of SEQ ID NOs: 13 and 23, wherein the anti-DLL3 agent is administered at a dose of from 10 mg to 100 mg twice every three weeks.

2. The method of claim 1, wherein the anti-DLL3 agent is administered at a dose of 10 mg, 30 mg, or 100 mg, twice every three weeks.

3. The method of claim 1 or claim 2, wherein the anti-DLL3 agent is administered on day 1 and day 8 of a 21-day cycle.

4. A method of treating DLL3-positive cancer, comprising administering to a subject in need thereof an anti-DLL3 agent comprising the amino acid sequence of SEQ ID NOs: 13 and 23, wherein the anti-DLL3 agent is administered at a dose of from 20 mg to 200 mg once every three weeks.

5. The method of claim 4, wherein the anti-DLL3 agent is administered at a dose of from 20 mg to 100 mg once every three weeks.

6. The method of claim 4, wherein the anti-DLL3 agent is administered at a dose of from 100 mg to 200 mg once every three weeks.

7. The method of any one of claims 4-6, wherein the anti-DLL3 agent is administered at a dose of 20 mg, 60 mg, 100 mg, or 200 mg.

8. The method of any one of claims 4-7, wherein the anti-DLL3 agent is administered on day one of a 21-day cycle.

9. A method of treating DLL3-positive cancer comprising administering to a subject in need thereof an anti-DLL3 agent comprising the amino acid sequence of SEQ ID NOs: 13 and 23, wherein the anti-DLL3 agent is administered according to the following regimen:
a) administering the anti-DLL3 agent in a first cycle wherein (i) the anti-DLL3 agent is administered at a dose of from 1 mg to 200 mg by continuous intravenous infusion over a period of 2 days to 7 days, and (ii) after the continuous intravenous infusion, the anti-DLL3 agent is administered by a bolus intravenous infusion on day 8, day 15, or both day 8 and day 15, and
b) administering the anti-DLL3 agent according to any one of i) to iii) below:
i) administering one or more subsequent doses of the anti-DLL3 agent at a dose of from 10 mg to 100 mg starting on day 29 and once every two weeks thereafter;
ii) administering one or more subsequent doses of the anti-DLL3 agent at a dose of from 10 mg to 100 mg starting on day 22 and twice every three weeks thereafter; and
iii) administering one or more subsequent doses of the anti-DLL3 agent at a dose of from 20 mg to 200 mg starting on day 22 and thereafter once every three weeks.

10. The method of claim 9, wherein a) the anti-DLL3 agent is administered at a dose of from 30 mg to 100 mg by continuous intravenous infusion over a period of 2 days, 3 days, 5 days or 7 days.

11. The method of claim 9 or 10, wherein a) the anti-DLL3 agent is administered at a dose of 30 mg, 50 mg, or 100 mg by continuous intravenous infusion over a period of 2 days, 3 days, 5 days or 7 days.

12. The method of any one of claims 9-11, wherein a) the anti-DLL3 agent is administered at a dose of 30 mg, 50 mg or 100 mg by continuous intravenous infusion over a period of 3 days, 5 days or 7 days.

13. The method of claim 12, wherein a) the anti-DLL3 agent is administered at a dose of 30 mg or 100 mg by continuous intravenous infusion over a period of 3 days.

14. A method of treating DLL3-positive cancer, comprising administering to a subject in need thereof an anti-DLL3 agent comprising the amino acid sequence of SEQ ID NOs: 13 and 23, an anti-PD-L1 antibody, and optionally one or more chemotherapeutic agents, wherein the anti-DLL3 agent is administered according to any one of a) to c) below:
a) administering the anti-DLL3 agent at a dose of from 10 mg to 100 mg once every two weeks;
b) administering the anti-DLL3 agent at a dose of from 10 mg to 100 mg twice every three weeks; and
c) administering the anti-DLL3 agent at a dose of from 20 mg to 200 mg once every three weeks.

15. The method of claim 14, wherein the anti-DLL3 agent is administered at a dose of from 10 mg to 100 mg once every two weeks.

16. The method of claim 15, wherein the anti-DLL3 agent is administered at a dose of 10 mg, 30 mg, 50 mg, or 100 mg once every two weeks.

17. The method of claim 15 or 16, wherein the anti-DLL3 agent is administered on day 1 and day 15 of a 28-day cycle.

18. The method of claim 14, wherein the anti-DLL3 agent is administered at a dose of from 10 mg to 100 mg twice every three weeks.

19. The method of claim 18, wherein the anti-DLL3 agent is administered at a dose of 10 mg, 30 mg, or 100 mg, twice every three weeks.

20. The method of claim 18 or 19, wherein the anti-DLL3 agent is administered on day 1 and day 8 of a 21-day cycle.

21. The method of claim 14, wherein the anti-DLL3 agent is administered at a dose of from 20 mg to 100 mg once every three weeks.

22. The method of claim 14, wherein the anti-DLL3 agent is administered at a dose of from 100 mg to 200 mg once every three weeks.

23. The method of claim 21 or 22, wherein the anti-DLL3 agent is administered at a dose of 20 mg, 60 mg, 100 mg, or 200 mg once every three weeks.

24. The method of any one of claims 21-23, wherein the anti-DLL3 agent is administered on day 1 of a 21-day cycle.

25. The method of any one of claims 14-24, wherein prior to any one of a) to c), the anti-DLL3 agent is administered in a 21-day cycle according to the following regimen: a first dose of 0 mg or 1 mg on day 1, a second dose of from 1 mg to 100 mg on day 8, and a third dose of from 10 mg to 200 mg on day 15.

26. The method of claim 25, wherein the first dose is 1 mg on day 1, the second dose is from 10 mg to 100 mg on day 8, and the third dose is from 10 mg to 100 mg on day 15.

27. The method of claim 25, wherein the first dose is 1 mg on day 1, the second dose is from 10 to 100 mg on day 8, and the third dose is from 20 to 200 on day 15.

28. The method of any one of claims 14-24, wherein prior to any one of a) to c), the anti-DLL3 agent is administered according to the following regimen in a 21-day cycle:
(i) a first dose of 1 mg on day 1, a second dose of from 10 mg to 100 mg on day 8; or
(ii) a first dose of 1 mg on day 8, a second dose of from 10 mg to 100 mg on day 15.

29. The method of claim 28, wherein the 21-day cycle is the first cycle the anti-DLL3 is administered to the subject and wherein the second dose in (i) or (ii) is from 10 mg to 50 mg, preferably 10 mg or 20 mg.

30. The method of any one of claims 14-29, wherein the anti-PD-L1 antibody is a PD-L1 blocking antibody.

31. The method of claim 30, wherein the anti-PD-L1 antibody is atezolizumab or durvalumab.

32. The method of any one of claims 14-31, wherein the one or more chemotherapeutic agents comprise a platinum-based chemotherapeutic agent, etoposide, or both.

33. The method of claim 32, wherein the platinum-based chemotherapeutic agent is carboplatin or cisplatin.

34. The method of any one of claims 14-33, wherein the anti-DLL3 agent is administered after the administration of the anti-PD-L1 antibody and the one or more chemotherapeutic agents when given on the same day.

35. A method of treating DLL3-positive cancer, comprising administering to a subject in need thereof an anti-DLL3 agent comprising the amino acid sequence of SEQ ID NOs: 13 and 23, and an alkylating agent, wherein the anti-DLL3 agent is administered to the subject at a dose of from 10 mg to 200 mg once every three weeks.

36. The method of claim 35, wherein the anti-DLL3 agent is administered at a dose of from 10 mg to 100 mg once every three weeks.

37. The method of claim 36, wherein the anti-DLL3 agent is administered at a dose of from 100 mg to 200 mg once every three weeks.

38. The method of any one of claims 35-37, wherein the anti-DLL3 agent is administered at a dose of 10 mg, 20 mg, 60 mg, 100 mg, or 200 mg.

39. The method of any one of claims 35-38, wherein the anti-DLL3 agent is administered on day 1 of a 21-day cycle.

40. A method of treating DLL3-positive cancer, comprising administering to a subject in need thereof an anti-DLL3 agent comprising the amino acid sequence of SEQ ID NOs: 13 and 23, and an alkylating agent, wherein the anti-DLL3 agent is administered to the subject according to the following: a first dose of 0 mg or 1 mg on day 1, a second dose of from 10 mg to 100 mg on day 8, a third dose of from 10 mg to 200 mg on day 15, and one or more subsequent doses of from 10 mg to 200 mg starting on day 22 and once every three weeks thereafter.

41. The method of claim 40, wherein the first dose is 1 mg, the second dose is from 10 mg to 100 mg, the third dose is from 10 mg to 200 mg, and the one or more subsequent doses are the same and are the same as the third dose.

42. The method of claim 40, wherein the first dose is 1 mg, the second dose is 20 mg, 60 mg, or 100 mg, the third dose is 20 mg, 60 mg, or 200 mg, and the one or more subsequent doses are the same and are each 20 mg, 60 mg or 200 mg.

43. The method of any one of claims 40-42, wherein the one or more subsequent doses are administered on day 1 of a 21-day cycle.

44. The method of any one of claims 35-43, wherein the alkylating agent is a platinum-based agent.

45. The method of claim 44, wherein the platinum-based agent is lurbinectedin.

46. The method of claim 45, wherein lurbinectedin is administered at a dose of from 2.0 mg/m² to 3.2 mg/m² once every three weeks.

47. The method of any one of claims 35-46, wherein the anti-DLL3 agent is administered after the administration of alkylating agent when given on the same day.

48. The method of any one of claims 1-47, wherein the anti-DLL3 agent comprises the amino acid sequence of SEQ ID NO: 14, 27 or 32.

49. The method of any one of claims 1-48, the method further comprises administering one or more additional therapeutic agent to the subject.

50. The method of claim 49, wherein the one or more additional therapeutic agent is a corticosteroid, saline, or an anti-IL6 antibody.

51. The method of claim 50, wherein the corticosteroid is dexamethasone.

52. The method of any one of claims 49-51, wherein the one or more additional therapeutic agent is administered in cycle 1 wherein the anti-DLL3 agent is administered.

53. The method of any one of claims 1-52, wherein the cancer is small cell lung cancer (SCLC).

54. The method of any one of claims 1-53, wherein the cancer is relapsed/refractory SCLC (RR SCLC) or extensive disease SCLC (ED SCLC).

55. The method of any one of claims 1-54, wherein the subject is a human.

56. The method of claim 55, wherein the subject had at least one prior treatment of the cancer and relapsed.

57. The method of claim 56, wherein the least one prior treatment of the cancer is platinum chemotherapy, etoposide, and optionally an anti-PD-L1 antibody.

58. The method of claim 55, wherein the subject had no prior systemic treatment of the cancer.

59. The method of any one of claims 1-58, wherein the anti-DLL3 agent is tarlatamab.


SEQUENCE LISTING (37 sequences)

SEQ ID NO: 1 - protein, 5 aa, synthetic construct
SYYWS

SEQ ID NO: 2 - protein, 16 aa, synthetic construct
YVYYSGTTNYNPSLKS

SEQ ID NO: 3 - protein, 10 aa, synthetic construct
IAVTGFYFDY

SEQ ID NO: 4 - protein, 12 aa, synthetic construct
RASQRVNNNYLA

SEQ ID NO: 5 - protein, 7 aa, synthetic construct
GASSRAT

SEQ ID NO: 6 - protein, 9 aa, synthetic construct
QQYDRSPLT

SEQ ID NO: 7 - protein, 118 aa, synthetic construct
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSS

SEQ ID NO: 8 - protein, 108 aa, synthetic construct
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 9 - protein, 241 aa, synthetic construct
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 10 - protein, 496 aa, synthetic construct
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 11 - protein, 118 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 12 - protein, 108 aa, synthetic construct
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 13 - protein, 241 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 14 - protein, 496 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 15 - protein, 14 aa, synthetic construct
GSSTGAVTSGNYPN

SEQ ID NO: 16 - protein, 7 aa, synthetic construct
GTKFLAP

SEQ ID NO: 17 - protein, 9 aa, synthetic construct
VLWYSNRWV

SEQ ID NO: 18 - protein, 5 aa, synthetic construct
KYAMN

SEQ ID NO: 19 - protein, 19 aa, synthetic construct
RIRSKYNNYATYYADSVKD

SEQ ID NO: 20 - protein, 14 aa, synthetic construct
HGNFGNSYISYWAY

SEQ ID NO: 21 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 22 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 23 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 24 - protein, 984 aa, synthetic construct
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 982 aa, synthetic construct
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 984 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 982 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 618 aa, synthetic construct
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 29 - protein, 40 aa, synthetic construct
SGVTCADGPCFNGGLCVGGADPDSAYICHCPPGFQGSNCE

SEQ ID NO: 30 - protein, 37 aa, synthetic construct
RVDRCSLQPCRNGGLCLDLGHALRCRCRAGFAGPRCE

SEQ ID NO: 31 - protein, 78 aa, synthetic construct
SGVTCADGPCFNGGLCVGGADPDSAYICHCPPGFQGSNCEKRVDRCSLQPCRNGGLCLDLGHALRCRCRAGFAGPRCE

SEQ ID NO: 32 - protein, 981 aa, synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 27 aa, synthetic construct
FPVHPDGASALPAAPPGLRPGDPQRYL

SEQ ID NO: 34 - protein, 505 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLTLSCAASSSSVSLLSLAWYRQAPGKKRELVAGISDDGSIVYMDSVKGRFTISRDNAKNSVYLQMNSLRAEDTAVYYCYAYSWITRSPYWGQGTLVTVSSHHHHHH

SEQ ID NO: 35 - protein, 505 aa, synthetic construct
EVQLVESGGGLVQPGGSLTLSCAASSSSVSLLSLAWYRQAPGKKRELVAGISDDGSIVYMDSVKGRFTISRDNAKNSVYLQMNSLRAEDTAVYYCYAYSWITRSPYWGQGTLVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 36 - protein, 705 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 707 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG